(19) United States
(12) Patent Application Publication
(10) Pub. No.: US 2022/0298212 A1
(43) Pub. Date: Sep. 22, 2022

(54) RECOMBINANT 4-1BB BINDING PROTEINS AND THEIR USE

(71) Applicant: MOLECULAR PARTNERS AG, Schlieren (CH)

(72) Inventors: Christian REICHEN, Zurich (CH); Alexander LINK, Zurich (CH); Julia HEPP, Zurich (CH); Victor LEVITSKY, Birmensdorf (CH)

(21) Appl. No.: 17/596,099

(22) PCT Filed: Jun. 3, 2020
(86) PCT No.: PCT/EP2020/065319

§ 371 Date: Dec. 2, 2021

(30) Foreign Application Priority Data
Jun. 4, 2019 (EP) 19178280.4

(51) Int. Cl. C07K 14/47

(52) U.S. Cl. CPC C07K 14/4702; C07K 2318/20; A61K 38/00

(57) ABSTRACT

The present disclosure relates to recombinant binding proteins comprising a designed ankyrin repeat domain with binding specificity for 4-1 BB (CD 137). In addition, the disclosure relates to nucleic acids encoding such binding proteins, pharmaceutical compositions comprising such binding proteins or nucleic acids, and the use of such binding proteins, nucleic acids or pharmaceutical compositions in methods for activating 4-1 BB in cells expressing 4-1 BB, e.g., tumor-localized T-lymphocytes, and for treating or diagnosing diseases, such as cancer, in a mammal, including a human.

### CROSS-REFERENCE TO RELATED APPLICATION

The present application claims the benefit of and priority from European patent application EP19178280 filed on 4 Jun. 2019 with the European Patent Office. The content of European patent application EP19178280 is incorporated herein by reference in its entirety, including all tables, figures, and claims.

### FIELD OF THE DISCLOSURE

The present invention relates to recombinant binding proteins comprising a designed ankyrin repeat domain with binding specificity for 4-1BB (CD137). In addition, the invention relates to nucleic acids encoding such binding proteins, pharmaceutical compositions comprising such binding proteins or nucleic acids, and the use of such binding proteins, nucleic acids or pharmaceutical compositions in methods for activating 4-1 BB in cells expressing 4-1 BB, e.g., tumor-localized T-lymphocytes, and for treating or diagnosing diseases, such as cancer, in a mammal, including a human.

### BACKGROUND

Immune checkpoint inhibitors have revolutionized the treatment of many cancers but there is a need for significant improvement to broaden their utility. Checkpoint inhibition (e.g. targeting CTLA-4, PD-1, or PD-L1) is highly effective with sustained clinical benefit in some patients, but most patients (70-80%) do not appear to benefit at all or benefit only minimally. T cell co-stimulation is considered a promising approach to increase the proportion of responders and the number of patients with long-term benefit (Morrissey et al. Clin Transl Sci, 2016, 9:89-104; Alsaab et al. Front. Pharmacol., 2017, 8:561).

TNF receptor (TNFR) family member 4-1 BB (CD137; TNFRSF9) is a key co-stimulatory receptor, and when engaged by its ligand or by agonistic antibodies, is involved in the regulation of certain lymphocyte responses. For example, ligation of 4-1 BB induces an activating signal in CD8-positive T cells and natural killer cells, resulting in increased proliferation, pro-inflammatory cytokine secretion, cytolytic function and antibody-dependent cell-mediated cytotoxicity. In addition, 4-1BB promotes establishment and maintenance of T-cell memory pools and has been suggested to have an inhibitory effect on the functional activity of regulatory T-cells (Tregs) (Wang et al. Immunol Rev. 2009, 229(1):192-215; Croft et al. Nat Rev Immunol, 2009, 9:271-285). Also in activated normal B cells, interaction of 4-1 BB with its ligand at the time of B cell receptor engagement stimulates proliferation and enhances survival (Zhang X, et al. J Immunol. 2010, 184(2):787-795).

Consistent with these costimulatory properties, 4-1 BB promotes both antiviral T cell responses (Lin et al. J Immunol. 2009, 182(2):934-47) and antitumor T cell responses (Lynch et al. Immunol Rev., 2008, 22: 277-286; Curran et al. PLoS One. 2011, 6(4):e19499).

The ligand that stimulates 4-1 BB (i.e. 4-1 BBL) is expressed on activated antigen-presenting cells (APCs), myeloid progenitor cells and hematopoietic stem cells. 4-1 BB is thought to undergo receptor trimerization upon binding to its trimeric ligand (4-1BBL) to stimulate cellular responses (Bitra A et al, J. Biol. Chem. 2018, 293(4):1317-1329; Chin et al, Nature Communications (2018) 9:4679).

4-1BB activation with agonistic molecules in pre-clinical models promotes control of tumor growth. For example, the addition of 4-1 BB agonists to in vitro cultures of B lymphoma cells with rituximab and NK cells resulted in increased lymphoma killing (Kohrt et al. Blood, 2011; 117(8):2423-2432). Moreover, 4-1BB agonist mAbs have been shown to increase costimulatory molecule expression and markedly enhance cytolytic T lymphocyte responses, resulting in anti-tumor efficacy in various models. Hereby, 4-1 BB agonist mAbs have demonstrated efficacy in prophylactic and therapeutic settings and have established durable anti-tumor protective T cell memory responses in tumor models related to monotherapy and when applied in combination with other immunomodulatory molecules (IL-2, IL-15, OX40, PD-1/PDL-1, CTLA-4, TIM-3), radiotherapy, chemotherapy, or tumor-specific vaccination (Chester et al. Cancer Immunol Immunother, 2016, 65:1243-1248, Lynch et al., Immunol Rev., 2008; 222:277-286). 4-1BB agonists also inhibit autoimmune reactions in a variety of autoimmunity models. For example, 4-1BB has been found to ameliorate autoimmunity in an array of animal models (Vinay et al. J Mol Med. 2006, 84(9):726-736), including collagen-induced arthritis (Seo et al. 2004, Nat Med. 10(10):1088-94) and systemic lupus erythematosus (Sun et al. 2002, Nat Med. 8(12):1405-13.)

Because of their potent activity in murine tumor models, agonist antibodies targeting 4-1 BB have entered clinical trials for, inter alia, melanoma and lymphoma. However, translation of the preclinical promise to clinical benefit for patients has not yet been achieved and in fact progress has been disappointing. A rather recent entry into the clinic is PRS-343, a bispecific HER2-CD137 targeting molecule (an antibody-anticalin conjugate), whose potential activity is limited to HER2-positive cancers. Utomilumab and urelumab, two 4-1 BB antibodies, are the clinically most advanced 4-1 BB activating therapeutic candidates. These two 4-1BB agonistic antibodies have been investigated in several clinical trials and disappointment has arisen from the production of liver toxicity by urelumab and from unsatisfactory therapeutic efficacy data for both. The liver toxicity caused by urelumab can be attributed to systemic 4-1 BB activation and the low therapeutic efficacy appears likely to be due to low potency activation of 4-1 BB in tumors. Urelumab produced dose limiting liver toxicity in early phase 1 monotherapy studies, resulting in the use of a safe but potentially sub-optimal dose for later studies (Segal et al. 2016, Clin. Cancer Res. 23 (8): 1929-36). The less potent (ca. 10-fold) utomilumab had fewer side effects and was not hepatotoxic, but also did not have convincing antitumor activity in a phase 1 monotherapy study (Chester et al. 2018, Blood, 131(1):49-57; Segal et al. 2018, Clin. Cancer Res. 24 (8):1816-1823).

Thus, there still remains a need for therapeutic and diagnostic approaches for the treatment and characterization of diseases, including cancer, benefitting from 4-1 BB-specific binding and activation.

### SUMMARY

The present invention provides recombinant binding proteins comprising a designed ankyrin repeat domain with binding specificity for 4-1 BB. Further provided are such binding proteins linked to one or more localizer molecules, which facilitate clustering-mediated activation of 4-1 BB by the binding proteins. In addition, the invention provides nucleic acids encoding such binding proteins and pharmaceutical compositions comprising such binding proteins or nucleic acids. The invention also provides the use of such binding proteins, nucleic acids or pharmaceutical compositions in methods for localized activation of 4-1 BB in 4-1 BB-expressing cells or tissue, such as tumor tissue, and for treating and diagnosing diseases, such as cancer, in a mammal, including a human.

In one aspect, the invention provides such a recombinant binding protein comprising an ankyrin repeat domain with binding specificity for 4-1 BB, wherein said ankyrin repeat domain comprises an amino acid sequence with at least 75% and up to 100% amino acid sequence identity with any one of the ankyrin repeat domains of SEQ ID NOs: 1 to 38, wherein G at position 1 and/or S at position 2 of said ankyrin repeat domains are optionally missing; and L at the second last position and/or N at the last position of said ankyrin repeat domains of SEQ ID NOs: 2 to 4, 6-19, 25-27, 33-38 are optionally substituted by A. As an example, in one particular embodiment, the 4-1BB-specific recombinant binding protein of the invention comprises the amino acid sequence of SEQ ID NO: 1.

In one aspect, the invention provides such a recombinant binding protein comprising an ankyrin repeat domain with binding specificity for 4-1 BB, wherein said ankyrin repeat domain comprises an ankyrin repeat module with at least 80% and up to 100% amino acid sequence identity with any one of the ankyrin repeat modules of SEQ ID NOs: 58 to 71. As an example, in one particular embodiment, the 4-1BB-specific recombinant binding protein of the invention comprises an ankyrin repeat domain with binding specificity for 4-1 BB, wherein said ankyrin repeat domain comprises an ankyrin repeat module with an amino acid sequence selected from SEQ ID NOs: 58 and 59. In one particular embodiment, the 4-1BB-specific recombinant binding protein of the invention comprises an ankyrin repeat domain with binding specificity for 4-1 BB, wherein said ankyrin repeat domain comprises an ankyrin repeat module with the amino acid sequence of SEQ ID NO: 58, and an ankyrin repeat module with the amino acid sequence of SEQ ID NO: 59.

In another aspect, the invention provides such 4-1BB-specific recombinant binding proteins, wherein the binding proteins further comprise a localizer molecule. The localizer molecule may be selected from molecules of different structural and functional classes. For example, a localizer may be a polypeptide binding domain, a cell surface receptor ligand or a fragment or variant thereof, an antibody or a fragment or variant thereof, or an antibody-like protein based on a scaffold. In one aspect of the invention, the localizer molecule is covalently bound to the 4-1 BB-specific recombinant binding protein. The covalent bond may be a peptide bond between the 4-1 BB-specific binding protein and a localizer peptide or polypeptide, resulting in a fusion protein. Alternatively, the localizer molecule may be covalently conjugated to the 4-1 BB-specific binding protein.

In one particular embodiment, a 4-1 BB-specific recombinant binding protein of the invention comprises an ankyrin repeat domain with binding specificity for 4-1 BB fused to a localizer with binding specificity for a localizer target protein relevant in cancer biology, such as, e.g., a tumor-associated antigen. As examples, in one particular embodiment, a 4-1 BB-specific recombinant binding protein of the invention comprises an ankyrin repeat domain with binding specificity for 4-1 BB fused to another ankyrin repeat domain with binding specificity for fibronectin extra domain B (ED-B), Tumor Antigen A (TAA), epidermal growth factor receptor (EGFR) or human epidermal growth factor receptor 2 (HER2).

In another aspect, the invention provides nucleic acids encoding the 4-1 BB-specific binding proteins of the invention and pharmaceutical compositions comprising a 4-1 BB-specific binding protein or nucleic acid of the invention and a pharmaceutically acceptable carrier and/or diluent.

In another aspect, the invention provides a method of localized activation of 4-1 BB in 4-1 BB-expressing cells or tissue in a mammal, the method comprising administering to said mammal a 4-1 BB-specific binding protein of the invention comprising a localizer molecule. In one particular embodiment, such method comprises administering the 4-1 BB-specific binding protein to a mammal, including a human patient, with a tumor comprising 4-1 BB-expressing cells or tissue, resulting in localized activation of 4-1 BB in the 4-1 BB-expressing cells or tumor tissue. In particular embodiments, such 4-1 BB-specific binding protein comprises the amino acid sequence of SEQ ID NO: 1 or SEQ ID NO: 5, or of sequence variants of SEQ ID NO: 1 or SEQ ID NO: 5 with equivalent 4-1 BB binding specificities.

In another aspect, the invention provides a method for treating a medical condition in a human patient, the method comprising administering to said patient a 4-1 BB-specific binding protein of the invention covalently linked to or comprising a localizer molecule, wherein the localizer molecule mediates local activation of 4-1 BB by the 4-1 BB-specific binding protein. In one particular embodiment, the medical condition is cancer, wherein the cancer or tumor tissue comprises cells that express 4-1 BB, and the localizer molecule binds a target overexpressed in said cancer or tumor tissue. In one particular embodiment, said target is the extracellular domain of a cell surface protein expressed or overexpressed in said cancer or tumor tissue. In one embodiment, said cancer is selected from colorectal cancers, gastric cancers, non-small cell lung cancers, breast cancers, head and neck cancer, ovarian cancers, lung cancers, invasive bladder cancers, pancreatic cancers, metastatic cancers of the brain, head and neck squamous cell carcinoma, esophagus squamous cell carcinoma, lung squamous cell carcinoma, skin squamous cell carcinoma, melanoma, breast adenocarcinoma, lung adenocarcinoma, cervix squamous cell carcinoma, pancreas squamous cell carcinoma, colon squamous cell carcinoma, or stomach squamous cell carcinoma, prostate cancer, osteosarcoma or soft tissue sarcoma and benign tumors. In one embodiment, such cancer is selected from epithelial malignancies (primary and metastatic), including lung, colorectal, gastric, bladder, ovarian and breast carcinomas, and bone and soft tissue sarcomas.

The invention further provides a kit comprising the recombinant binding protein of the invention, a nucleic acid of the invention or a pharmaceutical composition of the invention. The invention further provides a method for producing the recombinant binding protein of the invention, the method comprising the steps of (i) expressing said recombinant binding protein in bacteria, and (ii) purifying said recombinant binding protein using chromatography.

### BRIEF DESCRIPTION OF THE FIGURES

FIG. 1: SDS-PAGE gel analysis of the purification of two selected ankyrin repeat proteins with binding specificity for human 4-1 BB, DARPin® protein #1 and DARPin® protein #5. M corresponds to a protein size marker. The molecular weights (kDa) of the marker proteins are indicated. Lane 1: control ankyrin repeat protein with a molecular weight of 14.4 kDa; Lane 2: purified DARPin® protein #1; Lane 3: purified DARPin® protein #5.

FIGS. 2A to 2B: Surface Plasmon Resonance (SPR) analysis of ankyrin repeat protein binding to human 4-1BB, exemplified by DARPin® protein #1 (FIG. 2A) and DARPin® protein #5 (FIG. 2B). Various concentrations (0.6, 1.9, 5.6, and 16.7 nM) of purified ankyrin repeat protein were applied to a GLC chip with immobilized human 4-1 BB for on-rate and off-rate measurements. The obtained SPR trace analyses were used to determine the ankyrin repeat protein—4-1BB interaction. RU, Resonance Units; s, time in seconds.

FIG. 3: Schematic illustration of an assay to test the ability of ankyrin repeat proteins with binding specificity for 4-1 BB to activate 4-1 BB signaling in cells.

FIGS. 4A to 4D: Activation of 4-1 BB signaling by selected 4-1 BB-specific ankyrin repeat proteins, shown in a cellular reporter assay as illustrated in FIG. 3. Concentration-dependent curves are shown in relation to the determined luminescence (AU), which reflects reporter gene activity. An anti-4-1BB monoclonal antibody (20H4.9-IgG4) was tested as a comparison. FIG. 4A shows the curves for DARPin® protein #9, DARPin® protein #13, DARPin® protein #17 and the anti-4-1 BB monoclonal antibody. FIG. 4B shows the curves for DARPin® protein #1, DARPin® protein #2, DARPin® protein #3, DARPin® protein #10, DARPin® protein #11, DARPin® protein #14 and the anti-4-1BB monoclonal antibody. FIG. 4C shows the curves for DARPin® protein #5, DARPin® protein #6, DARPin® protein #12, DARPin® protein #15, DARPin® protein #18 and the anti-4-1 BB monoclonal antibody. FIG. 4D shows the curves for DARPin® protein #4, DARPin® protein #7, DARPin® protein #8, DARPin® protein #16, DARPin® protein #19 and the anti-4-1 BB monoclonal antibody.

FIG. 5: Binding of 4-1 BB-specific ankyrin repeat proteins to activated T lymphoblast cells (ionomycin/PMA stimulated CEM cells), as exemplified by DARPin® protein #44, DARPin® protein #45, DARPin® protein #46, DARPin® protein #47, DARPin® protein #48 and DARPin® protein #49. Concentration-dependent binding curves of these DARPin® proteins are shown in relation to the determined ratio of median fluorescence intensity (MFI) signal to background.

FIGS. 6A and 6B: Activation of 4-1 BB signaling by selected 4-1 BB-specific ankyrin repeat proteins, shown in 4-1 BB expressing cells cultured in the presence of TAA-coated beads. Concentration-dependent curves are shown in relation to the determined luminescence (AU), which reflects reporter gene activity. FIG. 6A shows the curves for DARPin® protein #44, DARPin® protein #45, DARPin® protein #46 and DARPin® protein #50. FIG. 6B shows the curves for DARPin® protein #1, DARPin® protein #2, DARPin® protein #47, DARPin® protein #48, and DARPin® protein #49. DARPin® protein #44, DARPin® protein #45, DARPin® protein #46, DARPin® protein #47, DARPin® protein #48, DARPin® protein #49 and DARPin® protein #50, but not DARPin® protein #1 or DARPin® protein #2, comprise a TAA-specific localizer molecule. FIG. 6A also shows results for the DARPin® proteins in the absence of TAA-coated beads.

FIGS. 7A and 7B: Activation of 4-1 BB signaling by selected 4-1 BB-specific ankyrin repeat proteins, shown in 4-1 BB expressing cells co-cultured with TAA-expressing CHO cells. Concentration-dependent curves are shown in relation to the determined luminescence (AU), which reflects reporter gene activity. FIG. 7A shows the curves for DARPin® protein #44, DARPin® protein #45, DARPin® protein #46, DARPin® protein #47, DARPin® protein #48, DARPin® protein #49 and DARPin® protein #50 in the presence of TAA-expressing CHO cells (Population 1). FIG. 7B shows the curves for DARPin® protein #44, DARPin® protein #45, DARPin® protein #46, DARPin® protein #47, DARPin® protein #48, DARPin® protein #49 and DARPin® protein #50 in the presence of another population of TAA-expressing CHO cells (Population 2). FIGS. 6A and 6B also show the luminescence values of the cells in the absence of any DARPin® protein.

FIGS. 8A to 8C: Binding of various ankyrin repeat proteins to activated T lymphoblast cells (ionomycin/PMA stimulated CEM cells). Concentration-dependent binding curves of the ankyrin repeat proteins are shown in relation to the determined ratio of median fluorescence intensity signal to background. FIG. 8A shows the curves for DARPin® protein #1, DARPin® protein #44, DARPin® protein #51 and DARPin® protein #54. FIG. 8B shows the curves for DARPin® protein #2, DARPin® protein #45, DARPin® protein #52 and DARPin® protein #55. FIG. 8C shows the curves for DARPin® protein #50, DARPin® protein #53 and DARPin® protein #56.

FIGS. 9A and 9B: Activation of 4-1BB signaling by selected 4-1BB-specific ankyrin repeat proteins, shown in 4-1BB expressing cells co-cultured with TAA-expressing CHO cells or with wild-type CHO cells. Concentration-dependent curves are shown in relation to the determined luminescence (AU), which reflects NF-KB-regulated reporter gene activity. FIG. 9A shows the curves for DARPin® protein #44, DARPin® protein #51 and DARPin® protein #54. FIG. 9B shows the curves for DARPin® protein #45, DARPin® protein #52 and DARPin® protein #55.

FIGS. 10A to 10C: ED-B expression in tumor stroma is shown by immunohistochemistry (FIG. 10A), and EGFR expression in A431 cells (FIG. 10B) and HER2 expression in BT474 cells (FIG. 10C) is shown by flow cytometry, using standard methods known in the art.

FIGS. 11A to 11C: Activation of 4-1BB signaling by selected 4-1BB-specific ankyrin repeat proteins, shown in 4-1BB expressing cells co-cultured with EGFR or HER2-expressing cells or in the presence of recombinant ED-B. Concentration-dependent curves are shown in relation to the determined luminescence (AU), which reflects reporter gene activity. FIG. 11A shows the curves for DARPin® protein #51, DARPin® protein #57, DARPin® protein #58 and DARPin® protein #59 in the presence of recombinant ED-B. FIG. 11B shows the curves for DARPin® protein #51 and DARPin® protein #58 in the presence of EGFR-expressing A431 cells. FIG. 11C shows the curves for DARPin® protein #58 and DARPin® protein #59 in the presence of HER2-expressing BT474 cells.

FIG. 12A and 12B: Activation of 4-1BB signaling by selected 4-1BB-specific ankyrin repeat proteins, shown in 4-1BB expressing primary CD8+ T-cells in the presence of EGFR-coated plates (FIG. 12A) or HER2-coated plates (FIG. 12B). Concentration-dependent curves are shown in relation to the determined secretion of interferon-γ (IFNγ), which is induced by 4-1 BB signaling. The curves represent DARPin® protein #58 and DARPin® protein #59.

### DETAILED DESCRIPTION OF THE INVENTION

As disclosed and exemplified herein, the disclosure provides ankyrin repeat proteins that specifically target 4-1 BB. Designed ankyrin repeat protein libraries (WO2002/020565; Binz et al., Nat. Biotechnol. 22, 575-582, 2004; Stumpp et al., Drug Discov. Today 13, 695-701, 2008) can be used for the selection of target-specific designed ankyrin repeat domains that bind to their target with high affinity. Such target-specific designed ankyrin repeat domains in turn can be used as valuable components of recombinant binding proteins for the treatment of diseases. Designed ankyrin repeat proteins are a class of binding molecules which have the potential to overcome limitations of monoclonal antibodies, hence allowing novel therapeutic approaches. Such ankyrin repeat proteins may comprise a single designed ankyrin repeat domain, or may comprise a combination of two or more designed ankyrin repeat domains with the same or different target specificities (Stumpp et al., Drug Discov. Today 13, 695-701, 2008; U.S. Pat. No. 9,458,211). Ankyrin repeat proteins comprising only a single designed ankyrin repeat domain are small proteins (14 kDa) which can be selected to bind a given target protein with high affinity and specificity. These characteristics, and the possibility of combining two or more designed ankyrin repeat domains in one protein, make designed ankyrin repeat proteins ideal agonistic, antagonistic and/or inhibitory drug candidates. Furthermore, such ankyrin repeat proteins can be engineered to carry various effector functions, e.g. cytotoxic agents or half-life extending agents, enabling completely new drug formats. Taken together, designed ankyrin repeat proteins are an example of the next generation of protein therapeutics with the potential to surpass existing antibody drugs.

DARPin® is a trademark owned by Molecular Partners AG, Switzerland.

In one aspect, the invention relates to a recombinant binding protein comprising an ankyrin repeat domain, wherein said ankyrin repeat domain has binding specificity for 4-1 BB, and wherein said ankyrin repeat domain comprises an ankyrin repeat module comprising an amino acid sequence selected from the group consisting of (1) SEQ ID NOs: 58 to 71 and (2) sequences in which up to 9, or up to 8, or up to 7, or up to 6, or up to 5, or up to 4, or up to 3, or up to 2, or up to 1 amino acids in any of SEQ ID NOs: 58 to 71 are substituted by another amino acid. Thus, in one embodiment, said ankyrin repeat module comprises an amino acid sequence selected from the group consisting of (1) SEQ ID NOs: 58 to 71 and (2) sequences in which up to 3 amino acids in any of SEQ ID NOs: 58 to 71 are substituted by another amino acid. In one embodiment, said ankyrin repeat module comprises an amino acid sequence selected from the group consisting of (1) SEQ ID NOs: 58 to 71 and (2) sequences in which up to 2 amino acids in any of SEQ ID

NOs: 58 to 71 are substituted by another amino acid. In one embodiment, said ankyrin repeat module comprises an amino acid sequence selected from the group consisting of (1) SEQ ID NOs: 58 to 71 and (2) sequences in which up to 1 amino acid in any of SEQ ID NOs: 58 to 71 is substituted by another amino acid. In one embodiment, all of said 9, 8, 7, 6, 5, 4, 3, 2, or 1 amino acid substitutions occur in framework positions of said ankyrin repeat module(s). In one embodiment, said ankyrin repeat module comprises an amino acid sequence selected from the group consisting of SEQ ID NOs: 58 to 71.

In one embodiment, said ankyrin repeat module comprises an amino acid sequence selected from the group consisting of (1) SEQ ID NOs: 58, 59, 67, 68 and 69 and (2) sequences in which up to 9, or up to 8, or up to 7, or up to 6, or up to 5, or up to 4, or up to 3, or up to 2, or up to 1 amino acids in any of SEQ ID NOs: SEQ ID NOs: 58, 59, 67, 68 and 69 are substituted by another amino acid. Thus, in one embodiment, said ankyrin repeat module comprises an amino acid sequence selected from the group consisting of (1) SEQ ID NOs: 58, 59, 67, 68 and 69 and (2) sequences in which up to 3 amino acids in any of SEQ ID NOs: SEQ ID NOs: 58, 59, 67, 68 and 69 are substituted by another amino acid. In one embodiment, said ankyrin repeat module comprises an amino acid sequence selected from the group consisting of (1) SEQ ID NOs: 58, 59, 67, 68 and 69 and (2) sequences in which up to 2 amino acids in any of SEQ ID NOs: SEQ ID NOs: 58, 59, 67, 68 and 69 are substituted by another amino acid. In one embodiment, said ankyrin repeat module comprises an amino acid sequence selected from the group consisting of (1) SEQ ID NOs: 58, 59, 67, 68 and 69 and (2) sequences in which up to 1 amino acid in any of SEQ ID NOs: SEQ ID NOs: 58, 59, 67, 68 and 69 is substituted by another amino acid. In one embodiment, all of said 9, 8, 7, 6, 5, 4, 3, 2, or 1 amino acid substitutions occur in framework positions of said ankyrin repeat module(s). In one embodiment, said ankyrin repeat module comprises an amino acid sequence selected from the group consisting of SEQ ID NOs: 58, 59, 67, 68 and 69.

In one embodiment, said ankyrin repeat module comprises the amino acid sequence of SEQ ID NO: 58 or a sequence in which one or two amino acids in SEQ ID NO: 58 are substituted by another amino acid. In one embodiment, said ankyrin repeat module comprises the amino acid sequence of SEQ ID NO: 59 or a sequence in which one or two amino acids in SEQ ID NO: 59 are substituted by another amino acid. In one embodiment, said ankyrin repeat module comprises the amino acid sequence of SEQ ID NO: 67 or a sequence in which one or two amino acids in SEQ ID NO: 67 are substituted by another amino acid. In one embodiment, said ankyrin repeat module comprises the amino acid sequence of SEQ ID NO: 68 or a sequence in which one or two amino acids in SEQ ID NO: 68 are substituted by another amino acid. In one embodiment, said ankyrin repeat module comprises the amino acid sequence of SEQ ID NO: 69 or a sequence in which one or two amino acids in SEQ ID NO: 69 are substituted by another amino acid. In one embodiment, said ankyrin repeat module comprises the amino acid sequence of SEQ ID NO: 58. In one embodiment, said ankyrin repeat module comprises the amino acid sequence of SEQ ID NO: 59. In one embodiment, said ankyrin repeat module comprises the amino acid sequence of SEQ ID NO: 67. In one embodiment, said ankyrin repeat module comprises the amino acid sequence of SEQ ID NO: 68. In one embodiment, said ankyrin repeat module comprises the amino acid sequence of SEQ ID NO: 69.

In one embodiment, all of said amino acid substitutions of said ankyrin repeat module(s) as described and referred to herein occur in framework positions of said ankyrin repeat module(s), wherein typically the overall structure of the module(s) is not affected by the substitutions. Such an embodiment of substitution in framework positions shall apply to all embodiments irrespective of whether such substitution is explicitly described.

In one embodiment, all of said amino acid substitutions of said ankyrin repeat module(s) as described and referred to herein occur in positions other than the randomized positions 3, 4, 6, 14 and 15 of said ankyrin repeat module(s) of SEQ ID NOs: 58 to 66 and 68 to 71 or the randomized positions 3, 5, 7, 15 and 16 of said ankyrin repeat module of SEQ ID NO: 67.

In one embodiment, said ankyrin repeat domain comprises a first ankyrin repeat module and a second ankyrin repeat module. In one embodiment, said first ankyrin repeat module is located N-terminally of said second ankyrin repeat module within said ankyrin repeat domain.

In one embodiment, said ankyrin repeat domain comprises a first ankyrin repeat module and a second ankyrin repeat module and a third ankyrin repeat module. In one embodiment, said first ankyrin repeat module is located N-terminally of said second ankyrin repeat module within said ankyrin repeat domain, and said second ankyrin repeat module is located N-terminally of said third ankyrin repeat module within said ankyrin repeat domain.

In one embodiment, said first, said second and, if present, said third ankyrin repeat module comprises an amino acid sequence selected from the group consisting of (1) SEQ ID NOs: 58 to 71 and (2) sequences in which up to 9, or up to 8, or up to 7, or up to 6, or up to 5, or up to 4, or up to 3, or up to 2, or up to 1 amino acids in any of SEQ ID NOs: 58 to 71 are substituted by another amino acid. In one embodiment, said first, said second and, if present, said third ankyrin repeat module comprises an amino acid sequence selected from the group consisting of (1) SEQ ID NOs: 58, 59, 67, 68 and 69 and (2) sequences in which up to 9, or up to 8, or up to 7, or up to 6, or up to 5, or up to 4, or up to 3, or up to 2, or up to 1 amino acids in any of SEQ ID NOs: SEQ ID NOs: 58, 59, 67, 68 and 69 are substituted by another amino acid.

In one embodiment, said ankyrin repeat domain comprises a first ankyrin repeat module and a second ankyrin repeat module. In one embodiment, in such an ankyrin repeat domain said first ankyrin repeat module comprises an amino acid sequence selected from the group consisting of (1) SEQ ID NO: 58 and (2) sequences in which up to 9, or up to 8, or up to 7, or up to 6, or up to 5, or up to 4, or up to 3, or up to 2, or up to 1 amino acids in SEQ ID NO: 58 are substituted by another amino acid, and said second ankyrin repeat module comprises an amino acid sequence selected from the group consisting of (1) SEQ ID NO: 59 and (2) sequences in which up to 9, or up to 8, or up to 7, or up to 6, or up to 5, or up to 4, or up to 3, or up to 2, or up to 1 amino acids of SEQ ID NO: 59 are substituted by another amino acid. In one embodiment, all of said 9, 8, 7, 6, 5, 4, 3, 2, or 1 amino acid substitutions occur in framework positions of said ankyrin repeat module(s), wherein typically the overall structure of the module(s) is not affected by the substitutions. In one embodiment, all of said 9, 8, 7, 6, 5, 4, 3, 2, or 1 amino acid substitutions occur in positions other than the randomized positions 3, 4, 6, 14 and 15 of said ankyrin repeat module(s).

In one embodiment, in such an ankyrin repeat domain said first ankyrin repeat module comprises an amino acid sequence selected from the group consisting of (1) SEQ ID NO: 58 and (2) sequences in which up to 6 amino acids in SEQ ID NO: 58 are substituted by another amino acid, and said second ankyrin repeat module comprises an amino acid sequence selected from the group consisting of (1) SEQ ID NO: 59 and (2) sequences in which up to 6 amino acids of SEQ ID NO: 59 are substituted by another amino acid. In one embodiment, in such an ankyrin repeat domain said first ankyrin repeat module comprises an amino acid sequence selected from the group consisting of (1) SEQ ID NO: 58 and (2) sequences in which up to 5 amino acids in SEQ ID NO: 58 are substituted by another amino acid, and said second ankyrin repeat module comprises an amino acid sequence selected from the group consisting of (1) SEQ ID NO: 59 and (2) sequences in which up to 5 amino acids of SEQ ID NO: 59 are substituted by another amino acid. In one embodiment, in such an ankyrin repeat domain said first ankyrin repeat module comprises an amino acid sequence selected from the group consisting of (1) SEQ ID NO: 58 and (2) sequences in which up to 4 amino acids in SEQ ID NO: 58 are substituted by another amino acid, and said second ankyrin repeat module comprises an amino acid sequence selected from the group consisting of (1) SEQ ID NO: 59 and (2) sequences in which up to 4 amino acids of SEQ ID NO: 59 are substituted by another amino acid. In one embodiment, in such an ankyrin repeat domain said first ankyrin repeat module comprises an amino acid sequence selected from the group consisting of (1) SEQ ID NO: 58 and (2) sequences in which up to 3 amino acids in SEQ ID NO: 58 are substituted by another amino acid, and said second ankyrin repeat module comprises an amino acid sequence selected from the group consisting of (1) SEQ ID NO: 59 and (2) sequences in which up to 3 amino acids of SEQ ID NO: 59 are substituted by another amino acid. In one embodiment, in such an ankyrin repeat domain said first ankyrin repeat module comprises an amino acid sequence selected from the group consisting of (1) SEQ ID NO: 58 and (2) sequences in which up to 2 amino acids in SEQ ID NO: 58 are substituted by another amino acid, and said second ankyrin repeat module comprises an amino acid sequence selected from the group consisting of (1) SEQ ID NO: 59 and (2) sequences in which up to 2 amino acids of SEQ ID NO: 59 are substituted by another amino acid. In one embodiment, in such an ankyrin repeat domain said first ankyrin repeat module comprises an amino acid sequence selected from the group consisting of (1) SEQ ID NO: 58 and (2) sequences in which 1 amino acid in SEQ ID NO: 58 is substituted by another amino acid, and said second ankyrin repeat module comprises an amino acid sequence selected from the group consisting of (1) SEQ ID NO: 59 and (2) sequences in which 1 amino acid of SEQ ID NO: 59 is substituted by another amino acid. In one embodiment, all of said amino acid substitutions occur in framework positions of said ankyrin repeat module(s), wherein typically the overall structure of the module(s) is not affected by the substitutions. In one embodiment, all of said amino acid substitutions occur in positions other than the randomized positions 3, 4, 6, 14 and 15 of said ankyrin repeat module(s). In one embodiment, in such an ankyrin repeat domain said first ankyrin repeat module comprises the amino acid sequence of SEQ ID NO: 58, and said second ankyrin repeat module comprises the amino acid sequence of SEQ ID NO: 59.

In one embodiment, said first ankyrin repeat module is located N-terminally of said second ankyrin repeat module within said ankyrin repeat domain. Thus, in one embodiment, said first ankyrin repeat module comprises an amino acid sequence selected from the group consisting of (1) SEQ ID NO: 58 and (2) sequences in which up to 3, or up to 2, or up to 1 amino acids in SEQ ID NO: 58 are substituted by another amino acid, and said second ankyrin repeat module comprises an amino acid sequence selected from the group consisting of (1) SEQ ID NO: 59 and (2) sequences in which up to 3, or up to 2, or up to 1 amino acids of SEQ ID NO: 59 are substituted by another amino acid, wherein said first ankyrin repeat module is located N-terminally of said second ankyrin repeat module within said ankyrin repeat domain. In one embodiment, all of said amino acid substitutions occur in framework positions of said ankyrin repeat module(s), wherein typically the overall structure of the module(s) is not affected by the substitutions. In one embodiment, all of said amino acid substitutions occur in positions other than the randomized positions 3, 4, 6, 14 and 15 of said ankyrin repeat module(s). Further, in one embodiment, said first ankyrin repeat module comprises the amino acid sequence of SEQ ID NO: 58 and said second ankyrin repeat module comprises the amino acid sequence of SEQ ID NO: 59, wherein said first ankyrin repeat module is located N-terminally of said second ankyrin repeat module within said ankyrin repeat domain.

In one embodiment, said ankyrin repeat domain comprises a first ankyrin repeat module and a second ankyrin repeat module and a third ankyrin repeat module. In one embodiment, in such an ankyrin repeat domain said first ankyrin repeat module comprises an amino acid sequence selected from the group consisting of (1) SEQ ID NO: 67 and (2) sequences in which up to 6 amino acids in SEQ ID NO: 67 are substituted by another amino acid, and said second ankyrin repeat module comprises an amino acid sequence selected from the group consisting of (1) SEQ ID NO: 68 and (2) sequences in which up to 6 amino acids of SEQ ID NO: 68 are substituted by another amino acid, and said third ankyrin repeat module comprises an amino acid sequence selected from the group consisting of (1) SEQ ID NO: 69 and (2) sequences in which up to 6 amino acids of SEQ ID NO: 69 are substituted by another amino acid. In one embodiment, in such an ankyrin repeat domain said first ankyrin repeat module comprises an amino acid sequence selected from the group consisting of (1) SEQ ID NO: 67 and (2) sequences in which up to 5 amino acids in SEQ ID NO: 67 are substituted by another amino acid, and said second ankyrin repeat module comprises an amino acid sequence selected from the group consisting of (1) SEQ ID NO: 68 and (2) sequences in which up to 5 amino acids of SEQ ID NO: 68 are substituted by another amino acid, and said third ankyrin repeat module comprises an amino acid sequence selected from the group consisting of (1) SEQ ID NO: 69 and (2) sequences in which up to 5 amino acids of SEQ ID NO: 69 are substituted by another amino acid. In one embodiment, in such an ankyrin repeat domain said first ankyrin repeat module comprises an amino acid sequence selected from the group consisting of (1) SEQ ID NO: 67 and (2) sequences in which up to 4 amino acids in SEQ ID NO: 67 are substituted by another amino acid, and said second ankyrin repeat module comprises an amino acid sequence selected from the group consisting of (1) SEQ ID NO: 68 and (2) sequences in which up to 4 amino acids of SEQ ID NO: 68 are substituted by another amino acid, and said third ankyrin repeat module comprises an amino acid sequence selected from the group consisting of (1) SEQ ID NO: 69 and (2) sequences in which up to 4 amino acids of SEQ ID NO: 69 are substituted by another amino acid. In one embodiment, in such an ankyrin repeat domain said first ankyrin repeat module comprises an amino acid sequence selected from the group consisting of (1) SEQ ID NO: 67 and (2) sequences in which up to 3 amino acids in SEQ ID NO: 67 are substituted by another amino acid, and said second ankyrin repeat module comprises an amino acid sequence selected from the group consisting of (1) SEQ ID NO: 68 and (2) sequences in which up to 3 amino acids of SEQ ID NO: 68 are substituted by another amino acid, and said third ankyrin repeat module comprises an amino acid sequence selected from the group consisting of (1) SEQ ID NO: 69 and (2) sequences in which up to 3 amino acids of SEQ ID NO: 69 are substituted by another amino acid. In one embodiment, in such an ankyrin repeat domain said first ankyrin repeat module comprises an amino acid sequence selected from the group consisting of (1) SEQ ID NO: 67 and (2) sequences in which up to 2 amino acids in SEQ ID NO: 67 are substituted by another amino acid, and said second ankyrin repeat module comprises an amino acid sequence selected from the group consisting of (1) SEQ ID NO: 68 and (2) sequences in which up to 2 amino acids of SEQ ID NO: 68 are substituted by another amino acid, and said third ankyrin repeat module comprises an amino acid sequence selected from the group consisting of (1) SEQ ID NO: 69 and (2) sequences in which up to 2 amino acids of SEQ ID NO: 69 are substituted by another amino acid. In one embodiment, in such an ankyrin repeat domain said first ankyrin repeat module comprises an amino acid sequence selected from the group consisting of (1) SEQ ID NO: 67 and (2) sequences in which 1 amino acid in SEQ ID NO: 67 is substituted by another amino acid, and said second ankyrin repeat module comprises an amino acid sequence selected from the group consisting of (1) SEQ ID NO: 68 and (2) sequences in which 1 amino acid of SEQ ID NO: 68 is substituted by another amino acid, and said third ankyrin repeat module comprises an amino acid sequence selected from the group consisting of (1) SEQ ID NO: 69 and (2) sequences in which 1 amino acid of SEQ ID NO: 69 is substituted by another amino acid.

In one embodiment, all of said amino acid substitutions occur in framework positions of said ankyrin repeat module(s), wherein typically the overall structure of the module(s) is not affected by the substitutions. In one embodiment, all of said amino acid substitutions occur in positions other than the randomized positions 3, 4, 6, 14 and 15 of said ankyrin repeat module(s) of SEQ ID NOs: 68 and 69 or the randomized positions 3, 5, 7, 15 and 16 of said ankyrin repeat module of SEQ ID NO: 67. In one embodiment, in such an ankyrin repeat domain said first ankyrin repeat module comprises the amino acid sequence of SEQ ID NO: 67, and said second ankyrin repeat module comprises the amino acid sequence of SEQ ID NO: 68, and said third ankyrin repeat module comprises the amino acid sequence of SEQ ID NO: 69.

In one embodiment, said first ankyrin repeat module is located N-terminally of said second ankyrin repeat module within said ankyrin repeat domain, and wherein said second ankyrin repeat module is located N-terminally of said third ankyrin repeat module within said ankyrin repeat domain. Thus, in one embodiment, said first ankyrin repeat module comprises an amino acid sequence selected from the group consisting of (1) SEQ ID NO: 67 and (2) sequences in which up to 3, or up to 2, or up to 1 amino acids in SEQ ID NO: 67 are substituted by another amino acid, and said second ankyrin repeat module comprises an amino acid sequence selected from the group consisting of (1) SEQ ID NO: 68 and (2) sequences in which up to 3, or up to 2, or up to 1 amino acids of SEQ ID NO: 68 are substituted by another amino acid, and said third ankyrin repeat module comprises an amino acid sequence selected from the group consisting of (1) SEQ ID NO: 69 and (2) sequences in which up to 3, or up to 2, or up to 1 amino acids of SEQ ID NO: 69 are substituted by another amino acid, wherein said first ankyrin repeat module is located N-terminally of said second ankyrin repeat module within said ankyrin repeat domain, and wherein said second ankyrin repeat module is located N-terminally of said third ankyrin repeat module within said ankyrin repeat domain. In one embodiment, all of said amino acid substitutions occur in framework positions of said ankyrin repeat module(s), wherein typically the overall structure of the module(s) is not affected by the substitutions. In one embodiment, all of said amino acid substitutions occur in positions other than the randomized positions 3, 4, 6, 14 and 15 of said ankyrin repeat module(s) of SEQ ID NOs: 68 and 69 or the randomized positions 3, 5, 7, 15 and 16 of said ankyrin repeat module of SEQ ID NO: 67. Further, in one embodiment, said first ankyrin repeat module comprises the amino acid sequence of SEQ ID NO: 67, and said second ankyrin repeat module comprises the amino acid sequence of SEQ ID NO: 68, and said third ankyrin repeat module comprises the amino acid sequence of SEQ ID NO: 69, wherein said first ankyrin repeat module is located N-terminally of said second ankyrin repeat module within said ankyrin repeat domain, and wherein said second ankyrin repeat module is located N-terminally of said third ankyrin repeat module within said ankyrin repeat domain.

In one embodiment, all of said amino acid substitutions in said ankyrin repeat module(s) described above occur in framework positions and in positions other than the randomized positions 3, 4, 6, 14 and 15 of said ankyrin repeat module(s) of SEQ ID NOs: 58 to 66 and 68 to 71 or the randomized positions 3, 5, 7, 15 and 16 of said ankyrin repeat module of SEQ ID NO: 67, wherein typically the overall structure of the module(s) is not affected by the substitutions.

In another aspect, the invention relates to a recombinant binding protein comprising an ankyrin repeat domain, wherein said ankyrin repeat domain has binding specificity for 4-1 BB, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 38, wherein G at position 1 and/or S at position 2 of SEQ ID NOs: 1 to 38 are optionally missing, and wherein L at the second last position and/or N at the last position of SEQ ID NOs: 2 to 4, 6 to 19, 25 to 27, and 33 to 38 are optionally substituted by A. Thus, in one embodiment, said ankyrin repeat domain comprises an amino acid sequence with at least 90% amino acid sequence identity with any one of SEQ ID NOs: 1 to 38. In another embodiment, said ankyrin repeat domain comprises an amino acid sequence with at least 93% amino acid sequence identity with any one of SEQ ID NOs: 1 to 38; and in a further embodiment, said ankyrin repeat domain comprises an amino acid sequence with at least 95% amino acid sequence identity with any one of SEQ ID NOs: 1 to 38. In one embodiment, said ankyrin repeat domain comprises an amino acid sequence with at least 98% amino acid sequence identity with any one of SEQ ID NOs: 1 to 38; and in one embodiment, said ankyrin repeat domain comprises the amino acid sequence of any one of SEQ ID NOs: 1 to 38.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1, 5, and 28 to 38, wherein G at position 1 and/or S at position 2 of SEQ ID NOs: 1, 5, and 28 to 38 are optionally missing, and wherein L at the second last position and/or N at the last position of SEQ ID NOs: 33 to 38 are optionally substituted by A. Thus, in one embodiment, said ankyrin repeat domain comprises an amino acid sequence with at least 90% amino acid sequence identity with any one of SEQ ID NOs: 1, 5, and 28 to 38. In another embodiment, said ankyrin repeat domain comprises an amino acid sequence with at least 93% amino acid sequence identity with any one of SEQ ID NOs: 1, 5, and 28 to 38; and in a further embodiment, said ankyrin repeat domain comprises an amino acid sequence with at least 95% amino acid sequence identity with any one of SEQ ID NOs: 1, 5, and 28 to 38. In one embodiment, said ankyrin repeat domain comprises an amino acid sequence with at least 98% amino acid sequence identity with any one of SEQ ID NOs: 1, 5, and 28 to 38; and in one embodiment, said ankyrin repeat domain comprises the amino acid sequence of any one of SEQ ID NOs: 1, 5, and 28 to 38. Thus, in one embodiment, said recombinant binding protein comprises an ankyrin repeat domain having binding specificity for 4-1 BB, wherein said ankyrin repeat domain comprises an amino acid sequence selected from SEQ ID NOs: 1, 5, and 28 to 38, wherein G at position 1 and/or S at position 2 of SEQ ID NOs: 1, 5, and 28 to 38 are optionally missing, and wherein L at the second last position and/or N at the last position of SEQ ID NOs: 33 to 38 are optionally substituted by A.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 1, wherein G at position 1 and/or S at position 2 of SEQ ID NO: 1 are optionally missing. Thus, in one embodiment, said ankyrin repeat domain comprises an amino acid sequence with at least 90% amino acid sequence identity with SEQ ID NO: 1. In another embodiment, said ankyrin repeat domain comprises an amino acid sequence with at least 93% amino acid sequence identity with SEQ ID NO: 1; and in a further embodiment, said ankyrin repeat domain comprises an amino acid sequence with at least 95% amino acid sequence identity with SEQ ID NO: 1. In one embodiment, said ankyrin repeat domain comprises an amino acid sequence with at least 98% amino acid sequence identity with SEQ ID NO: 1; and in one embodiment, said ankyrin repeat domain comprises the amino acid sequence of SEQ ID NO: 1. Thus, in one embodiment, said recombinant binding protein comprises an ankyrin repeat domain having binding specificity for 4-1 BB, wherein said ankyrin repeat domain comprises the amino acid sequence of SEQ ID NO: 1, wherein G at position 1 and/or S at position 2 of SEQ ID NO: 1 are optionally missing.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 5, wherein G at position 1 and/or S at position 2 of SEQ ID NO: 5 are optionally missing. Thus, in one embodiment, said ankyrin repeat domain comprises an amino acid sequence with at least 90% amino acid sequence identity with SEQ ID NO: 5. In another embodiment, said ankyrin repeat domain comprises an amino acid sequence with at least 93% amino acid sequence identity with SEQ ID NO: 5; and in a further embodiment, said ankyrin repeat domain comprises an amino acid sequence with at least 95% amino acid sequence identity with SEQ ID NO: 5. In one embodiment, said ankyrin repeat domain comprises an amino acid sequence with at least 98% amino acid sequence identity with SEQ ID NO: 5; and in one embodiment, said ankyrin repeat domain comprises the amino acid sequence of SEQ ID NO: 5. Thus, in one embodiment, said recombinant binding protein comprises an ankyrin repeat domain having binding specificity for 4-1 BB, wherein said ankyrin repeat domain comprises the amino acid sequence of SEQ ID NO: 5, wherein G at position 1 and/or S at position 2 of SEQ ID NO: 5 are optionally missing.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 28 to 38, wherein G at position 1 and/or S at position 2 of SEQ ID NOs: 28 to 38 are optionally missing, and wherein L at the second last position and/or N at the last position of SEQ ID NOs: 33 to 38 are optionally substituted by A. Thus, in one embodiment, said ankyrin repeat domain comprises an amino acid sequence with at least 90% amino acid sequence identity with any one of SEQ ID NOs: 28 to 38. In another embodiment, said ankyrin repeat domain comprises an amino acid sequence with at least 93% amino acid sequence identity with any one of SEQ ID NOs: 28 to 38; and in a further embodiment, said ankyrin repeat domain comprises an amino acid sequence with at least 95% amino acid sequence identity with any one of SEQ ID NOs: 28 to 38. In one embodiment, said ankyrin repeat domain comprises an amino acid sequence with at least 98% amino acid sequence identity with any one of SEQ ID NOs: 28 to 38; and in one embodiment, said ankyrin repeat domain comprises the amino acid sequence of any one of SEQ ID NOs: 28 to 38. Thus, in one embodiment, said recombinant binding protein comprises an ankyrin repeat domain having binding specificity for 4-1 BB, wherein said ankyrin repeat domain comprises an amino acid sequence selected from SEQ ID NOs: 28 to 38, wherein G at position 1 and/or S at position 2 of any one of SEQ ID NOs: 28 to 38 are optionally missing, and wherein L at the second last position and/or N at the last position of SEQ ID NOs: 33 to 38 are optionally substituted by A.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 31, wherein G at position 1 and/or S at position 2 of SEQ ID NO: 31 are optionally missing. Thus, in one embodiment, said ankyrin repeat domain comprises an amino acid sequence with at least 90% amino acid sequence identity with SEQ ID NO: 31.

In another embodiment, said ankyrin repeat domain comprises an amino acid sequence with at least 93% amino acid sequence identity with SEQ ID NO: 31; and in a further embodiment, said ankyrin repeat domain comprises an amino acid sequence with at least 95% amino acid sequence identity with SEQ ID NO: 31. In one embodiment, said ankyrin repeat domain comprises an amino acid sequence with at least 98% amino acid sequence identity with SEQ ID NO: 31; and in one embodiment, said ankyrin repeat domain comprises the amino acid sequence of SEQ ID NO: 31. Thus, in one embodiment, said recombinant binding protein comprises an ankyrin repeat domain having binding specificity for 4-1 BB, wherein said ankyrin repeat domain comprises the amino acid sequence of SEQ ID NO: 31, wherein G at position 1 and/or S at position 2 of SEQ ID NO: 31 are optionally missing.

In one embodiment, said recombinant binding protein comprises an ankyrin repeat domain having binding specificity for 4-1 BB, wherein the potential interaction residues in said ankyrin repeat domain are identical to the corresponding positions in any one of the ankyrin repeat domains of SEQ ID NOs: 1 to 27.

In one embodiment, said recombinant binding protein comprises an ankyrin repeat domain having binding specificity for 4-1 BB, wherein said ankyrin repeat domain binds human 4-1 BB in PBS with a dissociation constant (K_{D}) below 10⁻⁷M, or below 10⁻⁸M, or below 5×10⁻⁹M, or below 3×10⁻⁹M, or below 10⁻⁹M. Thus, in one embodiment, said ankyrin repeat domain binds human 4-1 BB in PBS with a dissociation constant (K_{D}) below 10⁻⁷M. In another embodiment, said ankyrin repeat domain binds human 4-1 BB in PBS with a dissociation constant (K_{D}) below 10⁻⁸M; and in a further embodiment, said ankyrin repeat domain binds human 4-1 BB in PBS with a dissociation constant (K_{D}) below 5×10⁻⁹M. In one embodiment, said ankyrin repeat domain binds human 4-1BB in PBS with a dissociation constant (K_{D}) below 3×10⁻⁹M. In one embodiment, said ankyrin repeat domain binds human 4-1 BB in PBS with a dissociation constant (K_{D}) below 10⁻⁹M.

In one embodiment, said recombinant binding protein comprises an ankyrin repeat domain having binding specificity for 4-1 BB, wherein said ankyrin repeat domain binds human 4-1 BB in PBS with a dissociation constant (K_{D}) below 10⁻⁷M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 38, wherein G at position 1 and/or S at position 2 of SEQ ID NOs: 1 to 38 are optionally missing, and wherein L at the second last position and/or N at the last position of SEQ ID NOs: 2 to 4, 6 to 19, 25 to 27, and 33 to 38 are optionally substituted by A. Thus, in one embodiment, said ankyrin repeat domain comprises an amino acid sequence with at least 90% amino acid sequence identity with any one of SEQ ID NOs: 1 to 38. In another embodiment, said ankyrin repeat domain comprises an amino acid sequence with at least 93% amino acid sequence identity with any one of SEQ ID NOs: 1 to 38; and in a further embodiment, said ankyrin repeat domain comprises an amino acid sequence with at least 95% amino acid sequence identity with any one of SEQ ID NOs: 1 to 38. In one embodiment, said ankyrin repeat domain comprises an amino acid sequence with at least 98% amino acid sequence identity with any one of SEQ ID NOs: 1 to 38; and in one embodiment, said ankyrin repeat domain comprises the amino acid sequence of any one of SEQ ID NOs: 1 to 38. Thus, in one embodiment, said recombinant binding protein comprises an ankyrin repeat domain having binding specificity for 4-1 BB, wherein said ankyrin repeat domain binds human 4-1 BB in PBS with a dissociation constant (K_{D}) below 10⁻⁷M, and wherein said ankyrin repeat domain comprises the amino acid sequence of any one of SEQ ID NOs: 1 to 38, wherein G at position 1 and/or S at position 2 of any one of SEQ ID NOs: 1 to 38 are optionally missing, and wherein L at the second last position and/or N at the last position of SEQ ID NOs: 2 to 4, 6 to 19, 25 to 27, and 33 to 38 are optionally substituted by A.

In one embodiment, said recombinant binding protein comprises an ankyrin repeat domain having binding specificity for 4-1 BB, wherein said ankyrin repeat domain binds human 4-1 BB in PBS with a dissociation constant (K_{D}) below 10⁻⁷M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 27, wherein G at position 1 and/or S at position 2 of SEQ ID NOs: 1 to 27 are optionally missing, and wherein L at the second last position and/or N at the last position of SEQ ID NOs: 2 to 4, 6 to 19, and 25 to 27 are optionally substituted by A. Thus, in one embodiment, said ankyrin repeat domain comprises an amino acid sequence with at least 90% amino acid sequence identity with any one of SEQ ID NOs: 1 to 27. In another embodiment, said ankyrin repeat domain comprises an amino acid sequence with at least 93% amino acid sequence identity with any one of SEQ ID NOs: 1 to 27; and in a further embodiment, said ankyrin repeat domain comprises an amino acid sequence with at least 95% amino acid sequence identity with any one of SEQ ID NOs: 1 to 27. In one embodiment, said ankyrin repeat domain comprises an amino acid sequence with at least 98% amino acid sequence identity with any one of SEQ ID NOs: 1 to 27; and in one embodiment, said ankyrin repeat domain comprises the amino acid sequence of any one of SEQ ID NOs: 1 to 27. Thus, in one embodiment, said recombinant binding protein comprises an ankyrin repeat domain having binding specificity for 4-1 BB, wherein said ankyrin repeat domain binds human 4-1 BB in PBS with a dissociation constant (K_{D}) below 10⁻⁷M, and wherein said ankyrin repeat domain comprises the amino acid sequence of any one of SEQ ID NOs: 1 to 27, wherein G at position 1 and/or S at position 2 of any one of SEQ ID NOs: 1 to 27 are optionally missing, and wherein L at the second last position and/or N at the last position of SEQ ID NOs: 2 to 4, 6 to 19, and 25 to 27 are optionally substituted by A. In one embodiment, said recombinant binding protein comprises an ankyrin repeat domain having binding specificity for 4-1 BB, wherein said ankyrin repeat domain binds human 4-1 BB in PBS with a dissociation constant (K_{D}) below 5×10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 14 and 16 to 27, wherein G at position 1 and/or S at position 2 of SEQ ID NOs: 1 to 14 and 16 to 27 are optionally missing, and wherein L at the second last position and/or N at the last position of SEQ ID NOs: 2 to 4, 6 to 14, 16 to 19 and 25 to 27 are optionally substituted by A. Thus, in one embodiment, said ankyrin repeat domain comprises an amino acid sequence with at least 90% amino acid sequence identity with any one of SEQ ID NOs: 1 to 14 and 16 to 27. In another embodiment, said ankyrin repeat domain comprises an amino acid sequence with at least 93% amino acid sequence identity with any one of SEQ ID NOs: 1 to 14 and 16 to 27; and in a further embodiment, said ankyrin repeat domain comprises an amino acid sequence with at least 95% amino acid sequence identity with any one of SEQ ID NOs: 1 to 14 and 16 to 27. In one embodiment, said ankyrin repeat domain comprises an amino acid sequence with at least 98% amino acid sequence identity with any one of SEQ ID NOs: 1 to 14 and 16 to 27; and in one embodiment, said ankyrin repeat domain comprises the amino acid sequence of any one of SEQ ID NOs: 1 to 14 and 16 to 27. Thus, in one embodiment, said recombinant binding protein comprises an ankyrin repeat domain having binding specificity for 4-1 BB, wherein said ankyrin repeat domain binds human 4-1 BB in PBS with a dissociation constant (K_{D}) below 5×10⁻⁹M, and wherein said ankyrin repeat domain comprises the amino acid sequence of any one of SEQ ID NOs: 1 to 14 and 16 to 27, wherein G at position 1 and/or Sat position 2 of any one of SEQ ID NOs: 1 to 14 and 16 to 27 are optionally missing, and wherein L at the second last position and/or N at the last position of SEQ ID NOs: 2 to 4, 6 to 14, 16 to 19, and 25 to 27 are optionally substituted by A.

In one embodiment, said recombinant binding protein comprises an ankyrin repeat domain having binding specificity for 4-1 BB, wherein said ankyrin repeat domain binds human 4-1 BB in PBS with a dissociation constant (K_{D}) below 3×10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 14 and 16 to 26, wherein G at position 1 and/or S at position 2 of SEQ ID NOs: 1 to 14 and 16 to 26 are optionally missing, and wherein L at the second last position and/or N at the last position of SEQ ID NOs: 2 to 4, 6 to 14, 16 to 19, and 25 to 26 are optionally substituted by A. Thus, in one embodiment, said ankyrin repeat domain comprises an amino acid sequence with at least 90% amino acid sequence identity with any one of SEQ ID NOs: 1 to 14 and 16 to 26. In another embodiment, said ankyrin repeat domain comprises an amino acid sequence with at least 93% amino acid sequence identity with any one of SEQ ID NOs: 1 to 14 and 16 to 26; and in a further embodiment, said ankyrin repeat domain comprises an amino acid sequence with at least 95% amino acid sequence identity with any one of SEQ ID NOs: 1 to 14 and 16 to 26. In one embodiment, said ankyrin repeat domain comprises an amino acid sequence with at least 98% amino acid sequence identity with any one of SEQ ID NOs: 1 to 14 and 16 to 26; and in one embodiment, said ankyrin repeat domain comprises the amino acid sequence of any one of SEQ ID NOs: 1 to 14 and 16 to 26. Thus, in one embodiment, said recombinant binding protein comprises an ankyrin repeat domain having binding specificity for 4-1 BB, wherein said ankyrin repeat domain binds human 4-1 BB in PBS with a dissociation constant (K_{D}) below 3×10⁻⁹M, and wherein said ankyrin repeat domain comprises the amino acid sequence of any one of SEQ ID NOs: 1 to 14 and 16 to 26, wherein G at position 1 and/or S at position 2 of any one of SEQ ID NOs: 1 to 14 and 16 to 26 are optionally missing, and wherein L at the second last position and/or N at the last position of SEQ ID NOs: 2 to 4, 6 to 14, 16 to 19, 25 to 26 are optionally substituted by A.

In one embodiment, said recombinant binding protein comprises an ankyrin repeat domain having binding specificity for 4-1 BB, wherein said ankyrin repeat domain binds human 4-1 BB in PBS with a dissociation constant (K_{D}) below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1, 2, 5, 7 to 14, 17, 20 to 24 and 26, wherein G at position 1 and/or S at position 2 of SEQ ID NOs: 1, 2, 5, 7 to 14, 17, 20 to 24 and 26 are optionally missing, and wherein L at the second last position and/or N at the last position of SEQ ID NOs: 2, 7 to 14, 17 and 26 are optionally substituted by A. Thus, in one embodiment, said ankyrin repeat domain comprises an amino acid sequence with at least 90% amino acid sequence identity with any one of SEQ ID NOs: 1, 2, 5, 7 to 14, 17, 20 to 24 and 26. In another embodiment, said ankyrin repeat domain comprises an amino acid sequence with at least 93% amino acid sequence identity with any one of SEQ ID NOs: 1, 2, 5, 7 to 14, 17, 20 to 24 and 26; and in a further embodiment, said ankyrin repeat domain comprises an amino acid sequence with at least 95% amino acid sequence identity with any one of SEQ ID NOs: 1, 2, 5, 7 to 14, 17, 20 to 24 and 26. In one embodiment, said ankyrin repeat domain comprises an amino acid sequence with at least 98% amino acid sequence identity with any one of SEQ ID NOs: 1, 2, 5, 7 to 14, 17, 20 to 24 and 26; and in one embodiment, said ankyrin repeat domain comprises the amino acid sequence of any one of SEQ ID NOs: 1, 2, 5, 7 to 14, 17, 20 to 24 and 26. Thus, in one embodiment, said recombinant binding protein comprises an ankyrin repeat domain having binding specificity for 4-1 BB, wherein said ankyrin repeat domain binds human 4-1 BB in PBS with a dissociation constant (K_{D}) below 10⁻⁹M, wherein said ankyrin repeat domain comprises the amino acid sequence of any one of SEQ ID NOs: 1, 2, 5, 7 to 14, 17, 20 to 24 and 26, wherein G at position 1 and/or S at position 2 of any one of SEQ ID NOs: 1, 2, 5, 7 to 14, 17, 20 to 24 and 26 are optionally missing, and wherein L at the second last position and/or N at the last position of SEQ ID NOs: 2, 7 to 14, 17 and 26 are optionally substituted by A.

In one embodiment, said recombinant binding protein comprises an ankyrin repeat domain having binding specificity for 4-1 BB, wherein said ankyrin repeat domain binds human 4-1 BB in PBS with a dissociation constant (K_{D}) below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1, 5, 7 to 11, 14, 17, 20 to 22 and 24, wherein G at position 1 and/or S at position 2 of SEQ ID NOs: 1, 5, 7 to 11, 14, 17, 20 to 22 and 24 are optionally missing, and wherein L at the second last position and/or N at the last position of SEQ ID NOs: 7 to 11, 14 and 17 are optionally substituted by A. Thus, in one embodiment, said ankyrin repeat domain comprises an amino acid sequence with at least 90% amino acid sequence identity with any one of SEQ ID NOs: 1, 5, 7 to 11, 14, 17, 20 to 22 and 24. In another embodiment, said ankyrin repeat domain comprises an amino acid sequence with at least 93% amino acid sequence identity with any one of SEQ ID NOs: 1, 5, 7 to 11, 14, 17, 20 to 22 and 24; and in a further embodiment, said ankyrin repeat domain comprises an amino acid sequence with at least 95% amino acid sequence identity with any one of SEQ ID NOs: 1, 5, 7 to 11, 14, 17, 20 to 22 and 24. In one embodiment, said ankyrin repeat domain comprises an amino acid sequence with at least 98% amino acid sequence identity with any one of SEQ ID NOs: 1, 5, 7 to 11, 14, 17, 20 to 22 and 24; and in one embodiment, said ankyrin repeat domain comprises the amino acid sequence of any one of SEQ ID NOs: 1, 5, 7 to 11, 14, 17, 20 to 22 and 24. Thus, in one embodiment, said recombinant binding protein comprises an ankyrin repeat domain having binding specificity for 4-1 BB, wherein said ankyrin repeat domain binds human 4-1 BB in PBS with a dissociation constant (K_{D}) below 10⁻⁹M, wherein said ankyrin repeat domain comprises the amino acid sequence of any one of SEQ ID NOs: 1, 5, 7 to 11, 14, 17, 20 to 22 and 24, wherein G at position 1 and/or Sat position 2 of any one of SEQ ID NOs: 1, 5, 7 to 11, 14, 17, 20 to 22 and 24 are optionally missing, and wherein L at the second last position and/or N at the last position of SEQ ID NOs: 7 to 11, 14 and 17 are optionally substituted by A.

In one embodiment, said recombinant binding protein comprises an ankyrin repeat domain having binding specificity for 4-1 BB, wherein said ankyrin repeat domain binds human 4-1 BB in PBS with a dissociation constant (K_{D}) below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 1, wherein G at position 1 and/or S at position 2 of SEQ ID NO: 1 are optionally missing. Thus, in one embodiment, said ankyrin repeat domain comprises an amino acid sequence with at least 90% amino acid sequence identity with SEQ ID NO: 1. In another embodiment, said ankyrin repeat domain comprises an amino acid sequence with at least 93% amino acid sequence identity with SEQ ID NO: 1; and in a further embodiment, said ankyrin repeat domain comprises an amino acid sequence with at least 95% amino acid sequence identity with SEQ ID NO: 1. In one embodiment, said ankyrin repeat domain comprises an amino acid sequence with at least 98% amino acid sequence identity with SEQ ID NO: 1; and in one embodiment, said ankyrin repeat domain comprises the amino acid sequence of SEQ ID NO: 1. Thus, in one embodiment, said recombinant binding protein comprises an ankyrin repeat domain having binding specificity for 4-1BB, wherein said ankyrin repeat domain binds human 4-1 BB in PBS with a dissociation constant (K_{D}) below 10⁻⁹M, wherein said ankyrin repeat domain comprises the amino acid sequence of SEQ ID NO: 1, wherein G at position 1 and/or S at position 2 of SEQ ID NO: 1 are optionally missing.

In one embodiment, said recombinant binding protein comprises an ankyrin repeat domain having binding specificity for 4-1 BB, wherein said ankyrin repeat domain binds human 4-1 BB in PBS with a dissociation constant (K_{D}) below 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 5, wherein G at position 1 and/or S at position 2 of SEQ ID NO: 5 are optionally missing. Thus, in one embodiment, said ankyrin repeat domain comprises an amino acid sequence with at least 90% amino acid sequence identity with

SEQ ID NO: 5. In another embodiment, said ankyrin repeat domain comprises an amino acid sequence with at least 93% amino acid sequence identity with SEQ ID NO: 5; and in a further embodiment, said ankyrin repeat domain comprises an amino acid sequence with at least 95% amino acid sequence identity with SEQ ID NO: 5. In one embodiment, said ankyrin repeat domain comprises an amino acid sequence with at least 98% amino acid sequence identity with SEQ ID NO: 5; and in one embodiment, said ankyrin repeat domain comprises the amino acid sequence of SEQ ID NO: 5. Thus, in one embodiment, said recombinant binding protein comprises an ankyrin repeat domain having binding specificity for 4-1BB, wherein said ankyrin repeat domain binds human 4-1 BB in PBS with a dissociation constant (K_{D}) below 10⁻⁹M, wherein said ankyrin repeat domain comprises the amino acid sequence of SEQ ID NO: 5, wherein G at position 1 and/or S at position 2 of SEQ ID NO: 5 are optionally missing.

A typical and preferred determination of dissociation constants (K_{D}) of the inventive recombinant binding proteins with binding specificity for 4-1 BB by Surface Plasmon Resonance (SPR) analysis is described in Example 2. Thus, in one embodiment said binding specificity for 4-1 BB of the inventive recombinant binding proteins is determined in PBS by Surface Plasmon Resonance (SPR). In one embodiment said binding specificity for 4-1 BB of the inventive recombinant binding proteins is determined in PBS by Surface Plasmon Resonance (SPR) as described in Example 2.

In one embodiment, said recombinant binding protein comprises two or three ankyrin repeat domains with binding specificity for 4-1 BB. In one embodiment, said recombinant binding protein comprises two or three ankyrin repeat domains with binding specificity for 4-1 BB, wherein each of said two or three ankyrin repeat domains independently comprises an ankyrin repeat module comprising an amino acid sequence selected from the group consisting of (1) SEQ ID NOs: 58 to 71 and (2) sequences in which up to 9, or up to 8, or up to 7, or up to 6, or up to 5, or up to 4, or up to 3, or up to 2, or up to 1 amino acids in any of SEQ ID NOs: 58 to 71 are substituted by another amino acid. Thus, in one embodiment, said recombinant binding protein comprises two or three ankyrin repeat domains with binding specificity for 4-1 BB, wherein each of said two or three ankyrin repeat domains independently comprises an ankyrin repeat module comprising an amino acid sequence selected from the group consisting of (1) SEQ ID NOs: 58 to 71 and (2) sequences in which up to 3 amino acids in any of SEQ ID NOs: 58 to 71 are substituted by another amino acid. In one embodiment, said ankyrin repeat module comprises an amino acid sequence selected from the group consisting of (1) SEQ ID NOs: 58 to 71 and (2) sequences in which up to 2 amino acids in any of SEQ ID NOs: 58 to 71 are substituted by another amino acid. In one embodiment, said ankyrin repeat module comprises an amino acid sequence selected from the group consisting of (1) SEQ ID NOs: 58 to 71 and (2) sequences in which up to 1 amino acid in any of SEQ ID NOs: 58 to 71 is substituted by another amino acid. In one embodiment, all of said 9, 8, 7, 6, 5, 4, 3, 2, or 1 amino acid substitutions occur in framework positions of said ankyrin repeat module(s), wherein typically the overall structure of the module(s) is not affected by the substitutions. In one embodiment, all of said amino acid substitutions occur in positions other than the randomized positions 3, 4, 6, 14 and 15 of said ankyrin repeat module(s) of SEQ ID NOs: 58 to 66 and 68 to 71 or the randomized positions 3, 5, 7, 15 and 16 of said ankyrin repeat module of SEQ ID NO: 67. In one embodiment, said recombinant binding protein comprises two or three ankyrin repeat domains with binding specificity for 4-1BB, wherein each of said two or three ankyrin repeat domains independently comprises an ankyrin repeat module comprising an amino acid sequence selected from the group consisting of SEQ ID NOs: 58 to 71. In one embodiment, said two or three ankyrin repeat domains are linked with a peptide linker. In one embodiment, said peptide linker is a proline-threonine rich peptide linker. In one embodiment, said peptide linker is the proline-threonine rich peptide linker of SEQ ID NO: 57.

In one embodiment, said two or three ankyrin repeat domains are linked with the proline-threonine rich peptide linker of SEQ ID NO: 57.

In one embodiment, said recombinant binding protein comprises two or three ankyrin repeat domains with binding specificity for 4-1 BB, wherein each of said two or three ankyrin repeat domains independently comprises an ankyrin repeat module comprising an amino acid sequence selected from the group consisting of (1) SEQ ID NOs: 58, 59, 67, 68 and 69 and (2) sequences in which up to 9, or up to 8, or up to 7, or up to 6, or up to 5, or up to 4, or up to 3, or up to 2, or up to 1 amino acids in any of SEQ ID NOs: 58, 59, 67, 68 and 69 are substituted by another amino acid. Thus, in one embodiment, said recombinant binding protein comprises two or three ankyrin repeat domains with binding specificity for 4-1 BB, wherein each of said two or three ankyrin repeat domains independently comprises an ankyrin repeat module comprising an amino acid sequence selected from the group consisting of (1) SEQ ID NOs: 58, 59, 67, 68 and 69 and (2) sequences in which up to 3 amino acids in any of SEQ ID NOs: 58, 59, 67, 68 and 69 are substituted by another amino acid. In one embodiment, said ankyrin repeat module comprises an amino acid sequence selected from the group consisting of (1) SEQ ID NOs: 58, 59, 67, 68 and 69 and (2) sequences in which up to 2 amino acids in any of SEQ ID NOs: 58, 59, 67, 68 and 69 are substituted by another amino acid. In one embodiment, said ankyrin repeat module comprises an amino acid sequence selected from the group consisting of (1) SEQ ID NOs: 58, 59, 67, 68 and 69 and (2) sequences in which up to 1 amino acid in any of SEQ ID NOs: 58, 59, 67, 68 and 69 is substituted by another amino acid. In one embodiment, all of said 9, 8, 7, 6, 5, 4, 3, 2, or 1 amino acid substitutions occur in framework positions of said ankyrin repeat module(s), wherein typically the overall structure of the module(s) is not affected by the substitutions. In one embodiment, all of said amino acid substitutions occur in positions other than the randomized positions 3, 4, 6, 14 and 15 of said ankyrin repeat module(s) of SEQ ID NOs: 58, 59, 68 and 69 or the randomized positions 3, 5, 7, 15 and 16 of said ankyrin repeat module of SEQ ID NO: 67. In one embodiment, said recombinant binding protein comprises two or three ankyrin repeat domains with binding specificity for 4-1 BB, wherein each of said two or three ankyrin repeat domains independently comprises an ankyrin repeat module comprising an amino acid sequence selected from the group consisting of SEQ ID NOs: 58, 59, 67, 68 and 69. In one embodiment, said two or three ankyrin repeat domains are linked with a peptide linker. In one embodiment, said peptide linker is a proline-threonine rich peptide linker. In one embodiment, said peptide linker is the proline-threonine rich peptide linker of SEQ ID NO: 57. In one embodiment, said two or three ankyrin repeat domains are linked with the proline-threonine rich peptide linker of SEQ ID NO: 57.

In one embodiment, said recombinant binding protein comprises two ankyrin repeat domains with binding specificity for 4-1 BB, wherein each of said two ankyrin repeat domains independently comprises an ankyrin repeat module comprising the amino acid sequence of (1) SEQ ID NO: 58 or (2) a sequence in which one or two amino acids in SEQ ID NO: 58 are substituted by another amino acid. In one embodiment, each of said two ankyrin repeat domains independently comprises an ankyrin repeat module comprising the amino acid sequence of (1) SEQ ID NO: 59 or (2) a sequence in which one or two amino acids in SEQ ID NO: 59 are substituted by another amino acid. In one embodiment, said recombinant binding protein comprises two ankyrin repeat domains with binding specificity for 4-1 BB, wherein each of said two ankyrin repeat domains independently comprises an ankyrin repeat module comprising the amino acid sequence of (1) SEQ ID NO: 67 or (2) a sequence in which one or two amino acids in SEQ ID NO: 67 are substituted by another amino acid. In one embodiment, each of said two ankyrin repeat domains comprises an ankyrin repeat module comprising the amino acid sequence of (1) SEQ ID NO: 68 or (2) a sequence in which one or two amino acids in SEQ ID NO: 68 are substituted by another amino acid. In one embodiment, each of said two ankyrin repeat domains independently comprises an ankyrin repeat module comprising the amino acid sequence of (1) SEQ ID NO: 69 or (2) a sequence in which one or two amino acids in SEQ ID NO: 69 are substituted by another amino acid. In one embodiment, all of said amino acid substitutions occur in framework positions, wherein typically the overall structure of the module(s) is not affected by the substitutions. In one embodiment, all of said amino acid substitutions occur in positions other than the randomized positions 3, 4, 6, 14 and 15 of said ankyrin repeat module(s) of SEQ ID NOs: 58, 59, 68 and 69 or the randomized positions 3, 5, 7, 15 and 16 of said ankyrin repeat module of SEQ ID NO: 67. In one embodiment, said two ankyrin repeat domains are linked with a peptide linker. In one embodiment, said peptide linker is a proline-threonine rich peptide linker. In one embodiment, said peptide linker is the proline-threonine rich peptide linker of SEQ ID NO: 57. In one embodiment, said two ankyrin repeat domains are linked with the proline-threonine rich peptide linker of SEQ ID NO: 57.

In one embodiment, all of said amino acid substitutions of said ankyrin repeat module(s) as described and referred to herein occur in framework positions of said ankyrin repeat module(s). Such an embodiment of substitution in framework positions shall apply to all embodiments irrespective of whether such substitution is explicitly described.

In one embodiment, said recombinant binding protein comprises two ankyrin repeat domains with binding specificity for 4-1 BB, wherein each of said two ankyrin repeat domains comprises an ankyrin repeat module comprising the amino acid sequence of SEQ ID NO: 58. In one embodiment, said recombinant binding protein comprises two ankyrin repeat domains with binding specificity for 4-1 BB, wherein each of said two ankyrin repeat domains comprises an ankyrin repeat module comprising the amino acid sequence of SEQ ID NO: 59. In one embodiment, said recombinant binding protein comprises two ankyrin repeat domains with binding specificity for 4-1 BB, wherein each of said two ankyrin repeat domains comprises an ankyrin repeat module comprising the amino acid sequence of SEQ ID NO: 67. In one embodiment, said recombinant binding protein comprises two ankyrin repeat domains with binding specificity for 4-1BB, wherein each of said two ankyrin repeat domains comprises an ankyrin repeat module comprising the amino acid sequence of SEQ ID NO: 68. In one embodiment, said recombinant binding protein comprises two ankyrin repeat domains with binding specificity for 4-1 BB, wherein each of said two ankyrin repeat domains comprises an ankyrin repeat module comprising the amino acid sequence of SEQ ID NO: 69.

In one embodiment, said recombinant binding protein comprises two ankyrin repeat domains with binding specificity for 4-1 BB, wherein each of said two ankyrin repeat domains comprises a first ankyrin repeat module and a second ankyrin repeat module. In one embodiment, said first ankyrin repeat module is located N-terminally of said second ankyrin repeat module within said ankyrin repeat domains.

In one embodiment, said recombinant binding protein comprises two ankyrin repeat domains with binding specificity for 4-1 BB, wherein each of said two ankyrin repeat domains comprises a first ankyrin repeat module and a second ankyrin repeat module, wherein said first ankyrin repeat module is located N-terminally of said second ankyrin repeat module within said ankyrin repeat domains, and wherein said first ankyrin repeat module comprises an amino acid sequence selected from the group consisting of (1) SEQ ID NO: 58 and (2) sequences in which up to 3, or up to 2, or up to 1 amino acids in SEQ ID NO: 58 are substituted by another amino acid, and wherein said second ankyrin repeat module comprises an amino acid sequence selected from the group consisting of (1) SEQ ID NO: 59 and (2) sequences in which up to 3, or up to 2, or up to 1 amino acids of SEQ ID NO: 59 are substituted by another amino acid. In one embodiment, said two ankyrin repeat domains are linked with a peptide linker. In one embodiment, said peptide linker is a proline-threonine rich peptide linker. In one embodiment, said peptide linker is the proline-threonine rich peptide linker of SEQ ID NO: 57. In one embodiment, said two ankyrin repeat domains are linked with the proline-threonine rich peptide linker of SEQ ID NO: 57. In one embodiment, said recombinant binding protein comprising two 4-1 BB specific ankyrin repeat domains binds human 4-1 BB in PBS with a dissociation constant (KO) below 10⁻⁹M.

Further, in one embodiment, said recombinant binding protein comprises two ankyrin repeat domains with binding specificity for 4-1 BB, wherein each of said two ankyrin repeat domains comprises a first ankyrin repeat module and a second ankyrin repeat module, wherein said first ankyrin repeat module comprises the amino acid sequence of SEQ ID NO: 58, and wherein said second ankyrin repeat module comprises the amino acid sequence of SEQ ID NO: 59, and wherein said first ankyrin repeat module is located N-terminally of said second ankyrin repeat module within said ankyrin repeat domain. In one embodiment, said recombinant binding protein comprising two 4-1 BB specific ankyrin repeat domains binds human 4-1 BB in PBS with a dissociation constant (KO) below 10⁻⁹M.

In one embodiment, said recombinant binding protein comprises two ankyrin repeat domains with binding specificity for 4-1 BB, wherein each of said two ankyrin repeat domains comprises a first ankyrin repeat module, a second ankyrin repeat module and a third ankyrin repeat module. In one embodiment, said first ankyrin repeat module is located N-terminally of said second ankyrin repeat module within said ankyrin repeat domains, and said second ankyrin repeat module is located N-terminally of said third ankyrin repeat module within said ankyrin repeat domains.

In one embodiment, said recombinant binding protein comprises two ankyrin repeat domains with binding specificity for 4-1 BB, wherein each of said two ankyrin repeat domains comprises a first ankyrin repeat module, a second ankyrin repeat module and a third ankyrin repeat module, wherein said first ankyrin repeat module is located N-terminally of said second ankyrin repeat module within said ankyrin repeat domains and said second ankyrin repeat module is located N-terminally of said third ankyrin repeat module within said ankyrin repeat domains, and wherein said first ankyrin repeat module comprises an amino acid sequence selected from the group consisting of (1) SEQ ID NO: 67 and (2) sequences in which up to 3, or up to 2, or up to 1 amino acids in SEQ ID NO: 67 are substituted by another amino acid, and wherein said second ankyrin repeat module comprises an amino acid sequence selected from the group consisting of (1) SEQ ID NO: 68 and (2) sequences in which up to 3, or up to 2, or up to 1 amino acids of SEQ ID NO: 68 are substituted by another amino acid, and wherein said third ankyrin repeat module comprises an amino acid sequence selected from the group consisting of (1) SEQ ID NO: 69 and (2) sequences in which up to 3, or up to 2, or up to 1 amino acids of SEQ ID NO: 69 are substituted by another amino acid. In one embodiment, said two ankyrin repeat domains are linked with a peptide linker. In one embodiment, said peptide linker is a proline-threonine rich peptide linker. In one embodiment, said peptide linker is the proline-threonine rich peptide linker of SEQ ID NO: 57. In one embodiment, said two ankyrin repeat domains are linked with the proline-threonine rich peptide linker of SEQ ID NO: 57. In one embodiment, said recombinant binding protein comprising two 4-1 BB specific ankyrin repeat domains binds human 4-1 BB in PBS with a dissociation constant (K_{D}) below 10⁻⁹M.

Further, in one embodiment, said recombinant binding protein comprises two ankyrin repeat domains with binding specificity for 4-1 BB, wherein each of said two ankyrin repeat domains comprises a first ankyrin repeat module, a second ankyrin repeat module and a third ankyrin repeat module, wherein said first ankyrin repeat module comprises the amino acid sequence of SEQ ID NO: 67, and wherein said second ankyrin repeat module comprises the amino acid sequence of SEQ ID NO: 68, and wherein said third ankyrin repeat module comprises the amino acid sequence of SEQ ID NO: 69, and wherein said first ankyrin repeat module is located N-terminally of said second ankyrin repeat module within said ankyrin repeat domains and said second ankyrin repeat module is located N-terminally of said third ankyrin repeat module within said ankyrin repeat domains. In one embodiment, said recombinant binding protein comprising two 4-1 BB specific ankyrin repeat domains binds human 4-1 BB in PBS with a dissociation constant (K_{D}) below 10⁻⁹M.

In one embodiment, said recombinant binding protein comprises two or three ankyrin repeat domains with binding specificity for 4-1BB, wherein each of said two or three ankyrin repeat domains independently comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 38, wherein G at position 1 and/or S at position 2 of SEQ ID NOs: 1 to 38 are optionally missing, and wherein L at the second last position and/or N at the last position of SEQ ID NOs: 2 to 4, 6 to 19, 25 to 27, and 33 to 38 are optionally substituted by A. Thus, in one embodiment, each of said two or three ankyrin repeat domains independently comprises an amino acid sequence with at least 90% amino acid sequence identity with any one of SEQ ID NOs: 1 to 38. In another embodiment, each of said two or three ankyrin repeat domains independently comprises an amino acid sequence with at least 93% amino acid sequence identity with any one of SEQ ID NOs: 1 to 38; and in a further embodiment, each of said two or three ankyrin repeat domains independently comprises an amino acid sequence with at least 95% amino acid sequence identity with any one of SEQ ID NOs: 1 to 38. In one embodiment, each of said two or three ankyrin repeat domains independently comprises an amino acid sequence with at least 98% amino acid sequence identity with any one of SEQ ID NOs: 1 to 38; and in one embodiment, each of said two or three ankyrin repeat domains independently comprises an amino acid sequence of any one of SEQ ID NOs: 1 to 38. In one embodiment, said two or three ankyrin repeat domains are linked with a peptide linker. In one embodiment, said peptide linker is a proline-threonine rich peptide linker. In one embodiment, said peptide linker is the proline-threonine rich peptide linker of SEQ ID NO: 57. In one embodiment, said two or three ankyrin repeat domains are linked with the proline-threonine rich peptide linker of SEQ ID NO: 57.

In one embodiment, said recombinant binding protein comprises two ankyrin repeat domains with binding specificity for 4-1BB, wherein each of said two ankyrin repeat domains independently comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 1, wherein G at position 1 and/or S at position 2 of SEQ ID NO: 1 are optionally missing. Thus, in one embodiment, each of said two ankyrin repeat domains independently comprises an amino acid sequence with at least 90% amino acid sequence identity with SEQ ID NO: 1. In another embodiment, each of said two ankyrin repeat domains independently comprises an amino acid sequence with at least 93% amino acid sequence identity with SEQ ID NO: 1; and in a further embodiment, each of said two ankyrin repeat domains independently comprises an amino acid sequence with at least 95% amino acid sequence identity with

SEQ ID NO: 1. In one embodiment, each of said two ankyrin repeat domains independently comprises an amino acid sequence with at least 98% amino acid sequence identity with SEQ ID NO: 1; and in one embodiment, each of said two ankyrin repeat domains comprises the amino acid sequence of SEQ ID NO: 1. Thus, in one embodiment, said recombinant binding protein comprises two ankyrin repeat domains with binding specificity for 4-1 BB, wherein each of said two ankyrin repeat domains comprises the amino acid sequence of SEQ ID NO: 1, wherein G at position 1 and/or S at position 2 of SEQ ID NO: 1 are optionally missing. In one embodiment, said two ankyrin repeat domains are linked with a peptide linker. In one embodiment, said peptide linker is a proline-threonine rich peptide linker. In one embodiment, said peptide linker is the proline-threonine rich peptide linker of SEQ ID NO:57. In one embodiment, said two ankyrin repeat domains are linked with the proline-threonine rich peptide linker of SEQ ID NO:57. In one embodiment, said recombinant binding protein comprising two 4-1 BB specific ankyrin repeat domains binds human 4-1 BB in PBS with a dissociation constant (K_{D}) below 10⁻⁹M.

In one embodiment, said recombinant binding protein comprises exactly two ankyrin repeat domains with binding specificity for 4-1 BB, wherein each of said two ankyrin repeat domains independently comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 1, wherein G at position 1 and/or S at position 2 of SEQ ID NO: 1 are optionally missing. Thus, in one embodiment, each of said two ankyrin repeat domains independently comprises an amino acid sequence with at least 90% amino acid sequence identity with SEQ ID NO: 1. In another embodiment, each of said two ankyrin repeat domains independently comprises an amino acid sequence with at least 93% amino acid sequence identity with SEQ ID NO: 1; and in a further embodiment, each of said two ankyrin repeat domains independently comprises an amino acid sequence with at least 95% amino acid sequence identity with SEQ ID NO: 1. In one embodiment, each of said two ankyrin repeat domains independently comprises an amino acid sequence with at least 98% amino acid sequence identity with SEQ ID NO: 1; and in one embodiment, each of said two ankyrin repeat domains comprises the amino acid sequence of SEQ ID NO: 1. Thus, in one embodiment, said recombinant binding protein comprises two ankyrin repeat domains with binding specificity for 4-1 BB, wherein each of said two ankyrin repeat domains comprises the amino acid sequence of SEQ ID NO: 1, wherein G at position 1 and/or S at position 2 of SEQ ID NO: 1 are optionally missing. In one embodiment, said two ankyrin repeat domains are linked with a peptide linker. In one embodiment, said peptide linker is a proline-threonine rich peptide linker. In one embodiment, said peptide linker is the proline-threonine rich peptide linker of SEQ ID NO:57. In one embodiment, said two ankyrin repeat domains are linked with the proline-threonine rich peptide linker of SEQ ID NO:57. In one embodiment, said recombinant binding protein comprising exactly two 4-1BB specific ankyrin repeat domains binds human 4-1 BB in PBS with a dissociation constant (K_{D}) below 10⁻⁹M.

In one embodiment, said recombinant binding protein comprises two ankyrin repeat domains with binding specificity for 4-1 BB, wherein each of said two ankyrin repeat domains independently comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 5, wherein G at position 1 and/or S at position 2 of SEQ ID NO: 5 are optionally missing. Thus, in one embodiment, each of said two ankyrin repeat domains independently comprises an amino acid sequence with at least 90% amino acid sequence identity with SEQ ID NO: 5. In another embodiment, each of said two ankyrin repeat domains independently comprises an amino acid sequence with at least 93% amino acid sequence identity with SEQ ID NO: 5; and in a further embodiment, each of said two ankyrin repeat domains independently comprises an amino acid sequence with at least 95% amino acid sequence identity with SEQ ID NO: 5. In one embodiment, each of said two ankyrin repeat domains independently comprises an amino acid sequence with at least 98% amino acid sequence identity with SEQ ID NO: 5; and in one embodiment, each of said two ankyrin repeat domains comprises the amino acid sequence of SEQ ID NO: 5. Thus, in one embodiment, said recombinant binding protein comprises two ankyrin repeat domains with binding specificity for 4-1 BB, wherein each of said two ankyrin repeat domains comprises the amino acid sequence of SEQ ID NO: 5, wherein G at position 1 and/or S at position 2 of SEQ ID NO: 5 are optionally missing. In one embodiment, said two ankyrin repeat domains are linked with a peptide linker. In one embodiment, said peptide linker is a proline-threonine rich peptide linker. In one embodiment, said peptide linker is the proline-threonine rich peptide linker of SEQ ID NO: 57. In one embodiment, said two ankyrin repeat domains are linked with the proline-threonine rich peptide linker of SEQ ID NO: 57. In one embodiment, said recombinant binding protein comprises exactly two ankyrin repeat domains with binding specificity for 4-1 BB. In one embodiment, said recombinant binding protein comprising two 4-1 BB specific ankyrin repeat domains binds human 4-1 BB in PBS with a dissociation constant (K_{D}) below 10⁻⁹M.

In one embodiment, said recombinant binding protein comprises a polypeptide consisting of two or three ankyrin repeat domains with binding specificity for 4-1BB linked with a peptide linker, wherein said polypeptide comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 51, 52, 54 and 55, wherein G at position 1 and/or S at position 2 of SEQ ID NOs: 51, 52, 54 and 55 are optionally missing, and wherein L at the second last position and/or N at the last position of each of said ankyrin repeat domains of SEQ ID NOs: 51, 52, 54 and 55 are optionally substituted by A. Thus, in one embodiment, said polypeptide comprises an amino acid sequence with at least 90% amino acid sequence identity with any one of SEQ ID NOs: 51, 52, 54 and 55. In another embodiment, said polypeptide comprises an amino acid sequence with at least 93% amino acid sequence identity with any one of SEQ ID NOs: 51, 52, 54 and 55; and in a further embodiment, said polypeptide comprises an amino acid sequence with at least 95% amino acid sequence identity with any one of SEQ ID NOs: 51, 52, 54 and 55. In one embodiment, said polypeptide comprises an amino acid sequence with at least 98% amino acid sequence identity with any one of SEQ ID NOs: 51, 52, 54 and 55; and in one embodiment, said polypeptide comprises the amino acid sequence of any one of SEQ ID NOs: 51, 52, 54 and 55. In one embodiment, said recombinant binding protein comprising two or three 4-1BB specific ankyrin repeat domains binds human 4-1 BB in PBS with a dissociation constant (K_{D}) below 10⁻⁹M.

In one embodiment, said recombinant binding protein comprises a polypeptide consisting of two ankyrin repeat domains with binding specificity for 4-1 BB linked with a peptide linker, wherein said polypeptide comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 51, wherein G at position 1 and/or S at position 2 of SEQ ID NO: 51 are optionally missing, and wherein L at the second last position and/or N at the last position of each of said ankyrin repeat domains of SEQ ID NO: 51 are optionally substituted by A. Thus, in one embodiment, said polypeptide comprises an amino acid sequence with at least 90% amino acid sequence identity with SEQ ID NO: 51. In another embodiment, said polypeptide comprises an amino acid sequence with at least 93% amino acid sequence identity with SEQ ID NO: 51; and in a further embodiment, said polypeptide comprises an amino acid sequence with at least 95% amino acid sequence identity with SEQ ID NO: 51. In one embodiment, said polypeptide comprises an amino acid sequence with at least 98% amino acid sequence identity with SEQ ID NO: 51. In one embodiment, said polypeptide comprises the amino acid sequence of SEQ ID NO: 51.

In one embodiment, said recombinant binding protein comprises a polypeptide consisting of two ankyrin repeat domains with binding specificity for 4-1BB linked with a peptide linker, wherein said polypeptide binds human 4-1BB in PBS with a dissociation constant (K_{D}) below 10⁻⁹M, or below 10⁻¹⁰M, or below 5×10⁻¹¹M. Thus, in one embodiment, said polypeptide binds human 4-1BB in PBS with a dissociation constant (K_{D}) below 10⁻⁹M. In a further embodiment, said polypeptide binds human 4-1 BB in PBS with a dissociation constant (K_{D}) below 10⁻¹⁰M. In another embodiment, said polypeptide binds human 4-1BB in PBS with a dissociation constant (K_{D}) below 5×10⁻¹¹M.

In one embodiment, said recombinant binding protein comprises a polypeptide consisting of two ankyrin repeat domains with binding specificity for 4-1BB linked with a peptide linker, wherein said polypeptide binds human 4-1 BB in PBS with a dissociation constant (K_{D}) below 10⁻¹⁰M, and wherein said polypeptide comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 51, wherein G at position 1 and/or S at position 2 of SEQ ID NO: 51 are optionally missing, and wherein L at the second last position and/or N at the last position of each of said ankyrin repeat domains of SEQ ID NO: 51 are optionally substituted by A. Thus, in one embodiment, said polypeptide comprises an amino acid sequence with at least 90% amino acid sequence identity with SEQ ID NO: 51. In another embodiment, said polypeptide comprises an amino acid sequence with at least 93% amino acid sequence identity with SEQ ID NO: 51; and in a further embodiment, said polypeptide comprises an amino acid sequence with at least 95% amino acid sequence identity with SEQ ID NO: 51. In one embodiment, said polypeptide comprises an amino acid sequence with at least 98% amino acid sequence identity with SEQ ID NO: 51.

Thus, in one embodiment, said recombinant binding protein comprises a polypeptide consisting of two ankyrin repeat domains with binding specificity for 4-1BB linked with a peptide linker, wherein said polypeptide binds human 4-1 BB in PBS with a dissociation constant (K_{D}) below 10⁻¹⁰M, and wherein said polypeptide comprises the amino acid sequence of SEQ ID NO: 51, wherein G at position 1 and/or S at position 2 of SEQ ID NO: 51 are optionally missing, and wherein L at the second last position and/or N at the last position of each of said ankyrin repeat domains of SEQ ID NO: 51 are optionally substituted by A.

Thus, in one embodiment, said recombinant binding protein comprises a polypeptide consisting of two ankyrin repeat domains with binding specificity for 4-1BB linked with a peptide linker, wherein said polypeptide binds human 4-1BB in PBS with a dissociation constant (K_{D}) below 5×10⁻¹¹M, and wherein said polypeptide comprises the amino acid sequence of SEQ ID NO: 51, wherein G at position 1 and/or S at position 2 of SEQ ID NO: 51 are optionally missing, and wherein L at the second last position and/or N at the last position of each of said ankyrin repeat domains of SEQ ID NO: 51 are optionally substituted by A.

In one embodiment, said recombinant binding protein further comprises a localizer molecule. In one embodiment, said localizer molecule is linked, conjugated, fused or otherwise physically attached to said 4-1 BB-specific ankyrin repeat domain or said two or three 4-1 BB-specific ankyrin repeat domains. In one embodiment, said localizer molecule is covalently linked to said 4-1 BB-specific ankyrin repeat domain or said two or three 4-1 BB-specific ankyrin repeat domains. In one embodiment, said localizer molecule is covalently linked to said 4-1 BB-specific ankyrin repeat domain or said two or three 4-1 BB-specific ankyrin repeat domains with a peptide linker. In one embodiment, said peptide linker is a proline-threonine rich peptide linker. In one embodiment, said peptide linker is the proline-threonine rich peptide linker of SEQ ID NO: 57. In one embodiment, said localizer molecule is covalently linked to said 4-1BB-specific ankyrin repeat domain or said two or three 4-1 BB-specific ankyrin repeat domains with the proline-threonine rich peptide linker of SEQ ID NO: 57.

As shown in Examples 4 to 6, one, two or three 4-1 BB-specific ankyrin repeat domains of the invention can be linked to a localizer that is capable of facilitating localized activation of 4-1 BB by the recombinant binding protein via, for example, localizer-mediated clustering. Such localized activation, e.g. targeted to tumor tissue, might be highly beneficial for avoiding or reducing systemic activation of 4-1 BB and resulting liver toxicity. Such embodiment allows to localize or deliver the activation of 4-1 BB to a specific tissue by way of said localizer molecule, which binds specifically, for example, to the extracellular domain of a molecule expressed selectively in cells of the target tissue and thereby clusters the 4-1 BB-specific ankyrin repeat domain or domains to achieve 4-1 BB activation in nearby 4-1 BB-expressing cells. For further example, numerous transmembrane proteins are known to be specifically expressed or over-expressed in tumor tissue in various types of cancers. Such tumor-specific proteins include, without limitation, anchoring proteins, receptors, enzymes, and transporter proteins such as NDC1 (TMEM48), TMEM45A, TMEM97, anoctamin-1 (TMEM16A), TMEM140, TMEM45B, αvβ3 integrin, Bombesin R, CAIX, CEA, CD13, CD44 v6, CXCR4, EGFR, ErbB-2, HER2, Emmprin, Endoglin, EpCAM, EphA2, fibronectin extra domain B (ED-B), FAP-α, Folate R, GRP78, IGF-1R, Matriptase, mesothelin, cMET/HGFR, MT1-MMP, MT6-MMP, Muc-1, PSCA, PSMA, Tn antigen, uPAR (Schmit K and Michiels C, Front. Pharmacol., 2018, 9:1345; Boonstra C M et al, Biomarkers in Cancer 2016, 8:119-133).

In one embodiment, said localizer molecule is a protein with binding specificity for a protein expressed in tumor tissue. In one embodiment, said localizer molecule is a protein with binding specificity for a tumor-specific protein. In one embodiment, said localizer molecule is a protein with binding specificity for a cell surface protein expressed in tumor tissue.

In one embodiment, said localizer molecule is an ankyrin repeat domain with binding specificity for a protein expressed in tumor tissue. In one embodiment, said localizer molecule is an ankyrin repeat domain with binding specificity for a tumor-specific protein. In one embodiment, said localizer molecule is an ankyrin repeat domain with binding specificity for a cell surface protein expressed in tumor tissue. In one embodiment, said localizer molecule is an ankyrin repeat domain with binding specificity for fibronectin extra domain B (ED-B), Tumor Antigen A (TAA), epidermal growth factor receptor (EGFR) or human epidermal growth factor receptor 2 (HER2).

In one embodiment, said recombinant binding protein comprises an ankyrin repeat domain having binding specificity for 4-1 BB and further comprises a localizer molecule, wherein said localizer molecule is a protein with binding specificity for a cell surface protein expressed in tumor tissue, and wherein said 4-1 BB-specific ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 38, wherein G at position 1 and/or S at position 2 of SEQ ID NOs: 1 to 38 are optionally missing, and wherein L at the second last position and/or N at the last position of SEQ ID NOs: 2 to 4, 6 to 19, 25 to 27, and 33 to 38 are optionally substituted by A. Thus, in one embodiment, said ankyrin repeat domain comprises an amino acid sequence with at least 90% amino acid sequence identity with any one of SEQ ID NOs: 1 to 38. In another embodiment, said ankyrin repeat domain comprises an amino acid sequence with at least 93% amino acid sequence identity with any one of SEQ ID NOs: 1 to 38; and in a further embodiment, said ankyrin repeat domain comprises an amino acid sequence with at least 95% amino acid sequence identity with any one of SEQ ID NOs: 1 to 38. In one embodiment, said ankyrin repeat domain comprises an amino acid sequence with at least 98% amino acid sequence identity with any one of SEQ ID NOs: 1 to 38; and in a further embodiment, said ankyrin repeat domain comprises the amino acid sequence of any one of SEQ ID NOs: 1 to 38. Thus, in one embodiment, said recombinant binding protein comprises an ankyrin repeat domain having binding specificity for 4-1 BB and further comprises a localizer molecule, wherein said localizer molecule is a protein with binding specificity for a cell surface protein expressed in tumor tissue, and wherein said ankyrin repeat domain comprises the amino acid sequence of any one of SEQ ID NOs: 1 to 38. In one embodiment, said localizer molecule is an ankyrin repeat domain.

In one embodiment, said recombinant binding protein comprises an ankyrin repeat domain having binding specificity for 4-1 BB and further comprises a localizer molecule, wherein said localizer molecule is a binding protein with binding specificity for a protein expressed in tumor tissue, and wherein said 4-1 BB-specific ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1, 5, and 28 to 38, wherein G at position 1 and/or S at position 2 of SEQ ID NOs: 1, 5, and 28 to 38 are optionally missing, and wherein L at the second last position and/or N at the last position of SEQ ID NOs: 33 to 38 are optionally substituted by A. In one embodiment, said localizer molecule is an ankyrin repeat domain.

In one embodiment, said recombinant binding protein comprises an ankyrin repeat domain having binding specificity for 4-1 BB and further comprises a localizer molecule, wherein said localizer molecule is a binding protein with binding specificity for a tumor-specific protein, and wherein said 4-1 BB-specific ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1, 5, and 28 to 38, wherein G at position 1 and/or S at position 2 of SEQ ID NOs: 1, 5, and 28 to 38 are optionally missing, and wherein L at the second last position and/or N at the last position of SEQ ID NOs: 33 to 38 are optionally substituted by A. In one embodiment, said localizer molecule is an ankyrin repeat domain.

In one embodiment, said recombinant binding protein comprises an ankyrin repeat domain having binding specificity for 4-1 BB and further comprises a localizer molecule, wherein said localizer molecule is a protein with binding specificity for a cell surface protein expressed in tumor tissue, and wherein said 4-1 BB-specific ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1, 5, and 28 to 38, wherein G at position 1 and/or S at position 2 of SEQ ID NOs: 1, 5, and 28 to 38 are optionally missing, and wherein L at the second last position and/or N at the last position of SEQ ID NOs: 33 to 38 are optionally substituted by A. In one embodiment, said localizer molecule is an ankyrin repeat domain.

In one embodiment, said recombinant binding protein comprises an ankyrin repeat domain having binding specificity for 4-1 BB and further comprises a localizer molecule, wherein said localizer molecule is a protein with binding specificity for a cell surface protein expressed in tumor tissue, and wherein said 4-1 BB-specific ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 1, wherein G at position 1 and/or S at position 2 of SEQ ID NO: 1 are optionally missing. In one embodiment, said localizer molecule is an ankyrin repeat domain.

In one embodiment, said recombinant binding protein comprises an ankyrin repeat domain having binding specificity for 4-1 BB and further comprises a localizer molecule, wherein said localizer molecule is a protein with binding specificity for a cell surface protein expressed in tumor tissue, and wherein said 4-1 BB-specific ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 5, wherein G at position 1 and/or S at position 2 of SEQ ID NO: 5 are optionally missing. In one embodiment, said localizer molecule is an ankyrin repeat domain.

In one embodiment, said recombinant binding protein comprises an ankyrin repeat domain having binding specificity for 4-1BB and further comprises a localizer molecule, wherein said localizer molecule is an ankyrin repeat domain with binding specificity for a cell surface protein expressed in tumor tissue, and wherein said 4-1 BB-specific ankyrin repeat domain comprises an amino acid sequence with at least 90%, 91%, 92%, 93%, 94%, 95%, 96%, 97%, 98%, 99%, or 100% amino acid sequence identity with SEQ ID NO: 1, wherein G at position 1 and/or S at position 2 of SEQ ID NO: 1 are optionally missing. Thus, in one embodiment, said 4-1 BB-specific ankyrin repeat domain comprises an amino acid sequence with at least 90% amino acid sequence identity with SEQ ID NO: 1. In another embodiment, said 4-1 BB-specific ankyrin repeat domain comprises an amino acid sequence with at least 93% amino acid sequence identity with SEQ ID NO: 1; and in a further embodiment, said 4-1 BB-specific ankyrin repeat domain comprises an amino acid sequence with at least 95% amino acid sequence identity with SEQ ID NO: 1. In one embodiment, said 4-1 BB-specific ankyrin repeat domain comprises an amino acid sequence with at least 98% amino acid sequence identity with SEQ ID NO: 1; and, in a further embodiment, said 4-1 BB-specific ankyrin repeat domain comprises the amino acid sequence of SEQ ID NO: 1. Thus, in one embodiment, said recombinant binding protein comprises an ankyrin repeat domain having binding specificity for 4-1 BB and further comprises a localizer molecule, wherein said localizer molecule is an ankyrin repeat domain with binding specificity for a cell surface protein expressed in tumor tissue, and wherein said 4-1 BB-specific ankyrin repeat domain comprises the amino acid sequence of SEQ ID NO: 1, wherein G at position 1 and/or S at position 2 of SEQ ID NO: 1 are optionally missing.

In one embodiment, said recombinant binding protein comprises an ankyrin repeat domain having binding specificity for 4-1BB and further comprises a localizer molecule, wherein said localizer molecule is an ankyrin repeat domain with binding specificity for a cell surface protein expressed in tumor tissue, and wherein said 4-1 BB-specific ankyrin repeat domain comprises an amino acid sequence with at least 90%, 91%, 92%, 93%, 94%, 95%, 96%, 97%, 98%, 99%, or 100% amino acid sequence identity with SEQ ID NO: 5, wherein G at position 1 and/or S at position 2 of SEQ ID NO: 5 are optionally missing. Thus, in one embodiment, said 4-1 BB-specific ankyrin repeat domain comprises an amino acid sequence with at least 90% amino acid sequence identity with SEQ ID NO: 5. In another embodiment, said 4-1 BB-specific ankyrin repeat domain comprises an amino acid sequence with at least 93% amino acid sequence identity with SEQ ID NO: 5; and in a further embodiment, said 4-1BB-specific ankyrin repeat domain comprises an amino acid sequence with at least 95% amino acid sequence identity with SEQ ID NO: 5. In one embodiment, said 4-1 BB-specific ankyrin repeat domain comprises an amino acid sequence with at least 98% amino acid sequence identity with SEQ ID NO: 5; and, in a further embodiment, said 4-1 BB-specific ankyrin repeat domain comprises the amino acid sequence of SEQ ID NO: 5. Thus, in one embodiment, said recombinant binding protein comprises an ankyrin repeat domain having binding specificity for 4-1 BB and further comprises a localizer molecule, wherein said localizer molecule is an ankyrin repeat domain with binding specificity for a cell surface protein expressed in tumor tissue, and wherein said 4-1 BB-specific ankyrin repeat domain comprises the amino acid sequence of SEQ ID NO: 5, wherein G at position 1 and/or S at position 2 of SEQ ID NO: 5 are optionally missing.

In one embodiment, said recombinant binding protein comprises an ankyrin repeat domain having binding specificity for 4-1 BB and further comprises a localizer molecule, wherein said 4-1 BB specific ankyrin repeat domain binds human 4-1BB in PBS with a dissociation constant (K_{D}) below 10⁻⁹M, wherein said localizer molecule is a protein with binding specificity for a cell surface protein expressed in tumor tissue, and wherein said 4-1 BB-specific ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1, 5, and 28 to 38, wherein G at position 1 and/or S at position 2 of SEQ ID NOs: 1, 5, and 28 to 38 are optionally missing, and wherein L at the second last position and/or N at the last position of SEQ ID NOs: 33 to 38 are optionally substituted by A. In one embodiment, said localizer molecule is an ankyrin repeat domain.

In one embodiment, said recombinant binding protein comprises an ankyrin repeat domain having binding specificity for 4-1 BB and further comprises a localizer molecule, wherein said 4-1 BB specific ankyrin repeat domain binds human 4-1 BB in PBS with a dissociation constant (K_{D}) below 10⁻⁹M, wherein said localizer molecule is an ankyrin repeat domain with binding specificity for a cell surface protein expressed in tumor tissue, and wherein said 4-1 BB-specific ankyrin repeat domain comprises an amino acid sequence with at least 90%, 91%, 92%, 93%, 94%, 95%, 96%, 97%, 98%, 99%, or 100% amino acid sequence identity with SEQ ID NO: 1, wherein G at position 1 and/or S at position 2 of SEQ ID NO: 1 are optionally missing. Thus, in one embodiment, said 4-1BB-specific ankyrin repeat domain comprises an amino acid sequence with at least 90% amino acid sequence identity with SEQ ID NO: 1. In another embodiment, said 4-1 BB-specific ankyrin repeat domain comprises an amino acid sequence with at least 93% amino acid sequence identity with SEQ ID NO: 1; and in a further embodiment, said 4-1 BB-specific ankyrin repeat domain comprises an amino acid sequence with at least 95% amino acid sequence identity with SEQ ID NO: 1. In one embodiment, said 4-1 BB-specific ankyrin repeat domain comprises an amino acid sequence with at least 98% amino acid sequence identity with SEQ ID NO: 1; and, in a further embodiment, said 4-1 BB-specific ankyrin repeat domain comprises the amino acid sequence of SEQ ID NO: 1. Thus, in one embodiment, said recombinant binding protein comprises an ankyrin repeat domain having binding specificity for 4-1 BB and further comprises a localizer molecule, wherein said 4-1BB specific ankyrin repeat domain binds human 4-1 BB in PBS with a dissociation constant (K_{D}) below 10⁻⁹M, wherein said localizer molecule is an ankyrin repeat domain with binding specificity for a cell surface protein expressed in tumor tissue, and wherein said 4-1 BB-specific ankyrin repeat domain comprises the amino acid sequence of SEQ ID NO: 1, wherein G at position 1 and/or S at position 2 of SEQ ID NO: 1 are optionally missing.

In one embodiment, said recombinant binding protein comprises an ankyrin repeat domain having binding specificity for 4-1 BB and further comprises a localizer molecule, wherein said 4-1 BB specific ankyrin repeat domain binds human 4-1 BB in PBS with a dissociation constant (K_{D}) below 10⁻⁹M, wherein said localizer molecule is an ankyrin repeat domain with binding specificity for a cell surface protein expressed in tumor tissue, and wherein said 4-1 BB-specific ankyrin repeat domain comprises an amino acid sequence with at least 90%, 91%, 92%, 93%, 94%, 95%, 96%, 97%, 98%, 99%, or 100% amino acid sequence identity with SEQ ID NO: 5, wherein G at position 1 and/or S at position 2 of SEQ ID NO: 5 are optionally missing. Thus, in one embodiment, said 4-1 BB-specific ankyrin repeat domain comprises an amino acid sequence with at least 90% amino acid sequence identity with SEQ ID NO: 5. In another embodiment, said 4-1 BB-specific ankyrin repeat domain comprises an amino acid sequence with at least 93% amino acid sequence identity with SEQ ID NO: 5; and in a further embodiment, said 4-1 BB-specific ankyrin repeat domain comprises an amino acid sequence with at least 95% amino acid sequence identity with SEQ ID NO: 5. In one embodiment, said 4-1 BB-specific ankyrin repeat domain comprises an amino acid sequence with at least 98% amino acid sequence identity with SEQ ID NO: 5; and, in a further embodiment, said 4-1 BB-specific ankyrin repeat domain comprises the amino acid sequence of SEQ ID NO: 5. Thus, in one embodiment, said recombinant binding protein comprises an ankyrin repeat domain having binding specificity for 4-1 BB and further comprises a localizer molecule, wherein said 4-1BB specific ankyrin repeat domain binds human 4-1 BB in PBS with a dissociation constant (K_{D}) below 10⁻⁹M, wherein said localizer molecule is an ankyrin repeat domain with binding specificity for a cell surface protein expressed in tumor tissue, and wherein said 4-1 BB-specific ankyrin repeat domain comprises the amino acid sequence of SEQ ID NO: 5, wherein G at position 1 and/or Sat position 2 of SEQ ID NO: 5 are optionally missing.

In one embodiment, said recombinant binding protein comprises two ankyrin repeat domains having binding specificity for 4-1BB and further comprises a localizer molecule, wherein said localizer molecule is an ankyrin repeat domain with binding specificity for a protein expressed in tumor tissue, and wherein each of said 4-1 BB specific ankyrin repeat domains independently comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1, 5, and 28 to 38, wherein G at position 1 and/or S at position 2 of SEQ ID NOs: 1, 5, and 28 to 38 are optionally missing, and wherein L at the second last position and/or N at the last position of SEQ ID NOs: 33 to 38 are optionally substituted by A.

In one embodiment, said recombinant binding protein comprises two ankyrin repeat domains having binding specificity for 4-1BB and further comprises a localizer molecule, wherein said localizer molecule is an ankyrin repeat domain with binding specificity for a tumor-specific protein, and wherein each of said 4-1 BB specific ankyrin repeat domains independently comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1, 5, and 28 to 38, wherein G at position 1 and/or S at position 2 of SEQ ID NOs: 1, 5, and 28 to 38 are optionally missing, and wherein L at the second last position and/or N at the last position of SEQ ID NOs: 33 to 38 are optionally substituted by A.

In one embodiment, said recombinant binding protein comprises two ankyrin repeat domains having binding specificity for 4-1BB and further comprises a localizer molecule, wherein said localizer molecule is an ankyrin repeat domain with binding specificity for a cell surface protein expressed in tumor tissue, and wherein each of said 4-1 BB specific ankyrin repeat domains independently comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1, 5, and 28 to 38, wherein G at position 1 and/or Sat position 2 of SEQ ID NOs: 1, 5, and 28 to 38 are optionally missing, and wherein L at the second last position and/or N at the last position of SEQ ID NOs: 33 to 38 are optionally substituted by A.

In one embodiment, said recombinant binding protein comprises two ankyrin repeat domains having binding specificity for 4-1 BB and further comprises a localizer molecule, wherein said recombinant binding protein binds human 4-1 BB in PBS with a dissociation constant (K_{D}) below 10⁻⁹M, wherein said localizer molecule is an ankyrin repeat domain with binding specificity for a cell surface protein expressed in tumor tissue, and wherein each of said 4-1 BB specific ankyrin repeat domains independently comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1, 5, and 28 to 38, wherein G at position 1 and/or Sat position 2 of SEQ ID NOs: 1, 5, and 28 to 38 are optionally missing, and wherein L at the second last position and/or N at the last position of SEQ ID NOs: 33 to 38 are optionally substituted by A.

In one embodiment, said recombinant binding protein comprises two ankyrin repeat domains having binding specificity for 4-1BB and further comprises a localizer molecule, wherein said localizer molecule is an ankyrin repeat domain with binding specificity for a protein expressed in tumor tissue, preferably for a cell surface protein expressed in tumor tissue, wherein said recombinant binding protein binds human 4-1 BB in PBS with a dissociation constant (K_{D}) below 10⁻⁹M, and wherein each of said 4-1 BB-specific ankyrin repeat domains independently comprises an amino acid sequence with at least 90% amino acid sequence identity with SEQ ID NO: 1, wherein G at position 1 and/or S at position 2 of SEQ ID NO: 1 are optionally missing. In one embodiment, said two 4-1BB-specific ankyrin repeat domains are linked with a proline-threonine rich peptide linker. In one embodiment, said peptide linker is the proline-threonine rich peptide linker of SEQ ID NO: 57. In one embodiment, said two 4-1BB-specific ankyrin repeat domains and said localizer ankyrin repeat domain are linked to each other with the proline-threonine rich peptide linker of SEQ ID NO: 57. In one embodiment, said localizer-specific ankyrin repeat domain is located N-terminally of said two 4-1 BB-specific ankyrin repeat domains within said recombinant binding protein.

In one embodiment, said recombinant binding protein comprises two ankyrin repeat domains having binding specificity for 4-1BB and further comprises a localizer molecule, wherein said localizer molecule is an ankyrin repeat domain with binding specificity for a cell surface protein expressed in tumor tissue, wherein said recombinant binding protein binds human 4-1 BB in PBS with a dissociation constant (K_{D}) below 10⁻¹⁰M, and wherein each of said 4-1 BB-specific ankyrin repeat domains independently comprises an amino acid sequence with at least 90% amino acid sequence identity with SEQ ID NO: 1, wherein G at position 1 and/or S at position 2 of SEQ ID NO: 1 are optionally missing. In one embodiment, said two 4-1 BB-specific ankyrin repeat domains are linked with a proline-threonine rich peptide linker. In one embodiment, said peptide linker is the proline-threonine rich peptide linker of SEQ ID NO: 57. In one embodiment, said two 4-1 BB-specific ankyrin repeat domains and said localizer ankyrin repeat domain are linked to each other with the proline-threonine rich peptide linker of SEQ ID NO: 57. In one embodiment, said localizer-specific ankyrin repeat domain is located N-terminally of said two 4-1 BB-specific ankyrin repeat domains within said recombinant binding protein.

In one embodiment, said recombinant binding protein comprises two ankyrin repeat domains having binding specificity for 4-1 BB and further comprises a localizer molecule, wherein said localizer molecule is an ankyrin repeat domain with binding specificity for a cell surface protein expressed in tumor tissue, wherein said recombinant binding protein binds human 4-1 BB in PBS with a dissociation constant (K_{D}) below 10⁻⁹M or below 10⁻¹⁰M, and wherein each of said 4-1 BB-specific ankyrin repeat domains independently comprises an amino acid sequence with at least 90% amino acid sequence identity with SEQ ID NO: 5, wherein G at position 1 and/or S at position 2 of SEQ ID NO: 5 are optionally missing. In one embodiment, said two 4-1BB-specific ankyrin repeat domains are linked with a proline-threonine rich peptide linker. In one embodiment, said peptide linker is the proline-threonine rich peptide linker of SEQ ID NO: 57. In one embodiment, said two 4-1 BB-specific ankyrin repeat domains and said localizer ankyrin repeat domain are linked to each other with the proline-threonine rich peptide linker of SEQ ID NO: 57. In one embodiment, said localizer-specific ankyrin repeat domain is located N-terminally of said two 4-1 BB-specific ankyrin repeat domains within said recombinant binding protein.

In one embodiment, said recombinant binding protein comprises a polypeptide consisting of two ankyrin repeat domains with binding specificity for 4-1BB linked with a peptide linker, and further comprises a localizer molecule, wherein said localizer molecule is an ankyrin repeat domain with binding specificity for a protein expressed in tumor tissue, preferably for a cell surface protein expressed in tumor tissue, wherein said polypeptide binds human 4-1 BB in PBS with a dissociation constant (K_{D}) below 10⁻⁹M, and wherein said polypeptide comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 51, wherein G at position 1 and/or S at position 2 of SEQ ID NO: 51 are optionally missing, and wherein L at the second last position and/or N at the last position of each of said ankyrin repeat domains of SEQ ID NO: 51 are optionally substituted by A. Thus, in one embodiment, said polypeptide comprises an amino acid sequence with at least 90% amino acid sequence identity with SEQ ID NO: 51. In another embodiment, said polypeptide comprises an amino acid sequence with at least 93% amino acid sequence identity with SEQ ID NO: 51; and in a further embodiment, said polypeptide comprises an amino acid sequence with at least 95% amino acid sequence identity with SEQ ID NO: 51. In one embodiment, said polypeptide comprises an amino acid sequence with at least 98% amino acid sequence identity with SEQ ID NO: 51; and in a further embodiment, said polypeptide comprises the amino acid sequence of SEQ ID NO: 51. In one embodiment, said polypeptide and said localizer ankyrin repeat domain are linked with a proline-threonine rich peptide linker. In one embodiment, said polypeptide and said localizer ankyrin repeat domain are linked with the proline-threonine rich peptide linker of SEQ ID NO: 57. In one embodiment, said localizer ankyrin repeat domain is located N-terminally of said polypeptide within said recombinant binding protein.

In one embodiment, said recombinant binding protein comprises a polypeptide consisting of two ankyrin repeat domains with binding specificity for 4-1BB linked with a peptide linker, and further comprises a localizer molecule, wherein said localizer molecule is an ankyrin repeat domain with binding specificity for a cell surface protein expressed in tumor tissue, wherein said polypeptide binds human 4-1 BB in PBS with a dissociation constant (K_{D}) below 10⁻¹⁰M, and wherein said polypeptide comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 51, wherein G at position 1 and/or S at position 2 of SEQ ID NO: 51 are optionally missing, and wherein L at the second last position and/or N at the last position of each of said ankyrin repeat domains of SEQ ID NO: 51 are optionally substituted by A. Thus, in one embodiment, said polypeptide comprises an amino acid sequence with at least 90% amino acid sequence identity with SEQ ID NO: 51. In another embodiment, said polypeptide comprises an amino acid sequence with at least 93% amino acid sequence identity with SEQ ID NO: 51; and in a further embodiment, said polypeptide comprises an amino acid sequence with at least 95% amino acid sequence identity with SEQ ID NO: 51. In one embodiment, said polypeptide comprises an amino acid sequence with at least 98% amino acid sequence identity with SEQ ID NO: 51; and in a further embodiment, said polypeptide comprises the amino acid sequence of SEQ ID NO: 51. In one embodiment, said polypeptide and said localizer ankyrin repeat domain are linked with a proline-threonine rich peptide linker. In one embodiment, said polypeptide and said localizer ankyrin repeat domain are linked with the proline-threonine rich peptide linker of SEQ ID NO: 57. In one embodiment, said localizer ankyrin repeat domain is located N-terminally of said polypeptide within said recombinant binding protein.

In one embodiment, said recombinant binding protein comprises two ankyrin repeat domains having binding specificity for 4-1 BB and further comprises a localizer molecule, wherein each of said two 4-1 BB-specific ankyrin repeat domains independently comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1, 5, and 31, wherein G at position 1 and/or S at position 2 of SEQ ID NOs: 1, 5, and 31 are optionally missing.

In one embodiment, said recombinant binding protein comprises two ankyrin repeat domains having binding specificity for 4-1 BB and further comprises a localizer molecule, wherein each of said two 4-1 BB-specific ankyrin repeat domains independently comprises an amino acid sequence with at least 90% amino acid sequence identity with any one of SEQ ID NOs: 1, 5, and 31, wherein G at position 1 and/or S at position 2 of SEQ ID NOs: 1, 5, and 31 are optionally missing, and wherein said localizer molecule is an ankyrin repeat domain with binding specificity for a cell surface protein expressed in tumor tissue. In one embodiment, said two 4-1BB-specific ankyrin repeat domains are linked with a peptide linker. In one embodiment, said peptide linker is a proline-threonine rich peptide linker. In one embodiment, said peptide linker is the proline-threonine rich peptide linker of SEQ ID NO: 57. In one embodiment, said two 4-1 BB-specific ankyrin repeat domains and said ankyrin repeat domain with binding specificity for a cell surface protein expressed in tumor tissue are linked to each other with the proline-threonine rich peptide linker of SEQ ID NO:57. In one embodiment, said ankyrin repeat domain with binding specificity for a cell surface protein expressed in tumor tissue is located N-terminally of said two 4-1 BB specific ankyrin repeat domains within said recombinant binding protein.

In one embodiment, said recombinant binding protein comprises two ankyrin repeat domains having binding specificity for 4-1 BB and further comprises a localizer molecule, wherein each of said 4-1 BB-specific ankyrin repeat domains independently comprises an amino acid sequence with at least 90% amino acid sequence identity with SEQ ID NO: 1, wherein G at position 1 and/or S at position 2 of SEQ ID NO: 1 are optionally missing. In one embodiment, said two 4-1 BB-specific ankyrin repeat domains are linked with a peptide linker. In one embodiment, said peptide linker is a proline-threonine rich peptide linker. In one embodiment, said peptide linker is the proline-threonine rich peptide linker of SEQ ID NO: 57. In one embodiment, said two 4-1 BB specific ankyrin repeat domains and said localizer molecule are linked to each other with the proline-threonine rich peptide linker of SEQ ID NO: 57. In one embodiment, said localizer molecule is located N-terminally of said two 4-1 BB specific ankyrin repeat domains within said recombinant binding protein.

In one embodiment, said 4-1 BB-specific recombinant binding protein of the invention further comprises an ankyrin repeat domain with binding specificity for serum albumin. In one embodiment, said 4-1 BB-specific recombinant binding protein of the invention further comprises two ankyrin repeat domains with binding specificity for serum albumin. In one embodiment, said 4-1 BB-specific recombinant binding protein of the invention further comprises two ankyrin repeat domains with binding specificity for serum albumin, wherein one of said two ankyrin repeat domains with binding specificity for serum albumin is located N-terminally of the 4-1 BB-specific ankyrin repeat domain and wherein the other one of said two ankyrin repeat domains with binding specificity for serum albumin is located C-terminally of the 4-1BB-specific ankyrin repeat domain. In one embodiment, said recombinant binding protein of the invention comprises two ankyrin repeat domains with binding specificity for 4-1 BB and further comprises two ankyrin repeat domains with binding specificity for serum albumin, wherein one of said two ankyrin repeat domains with binding specificity for serum albumin is located N-terminally of said 4-1 BB-specific ankyrin repeat domains and wherein the other one of said two ankyrin repeat domains with binding specificity for serum albumin is located C-terminally of said two 4-1 BB-specific ankyrin repeat domains. Ankyrin repeat domains with binding specificity for serum albumin are able to increase the in vivo half-life of the recombinant protein of the invention.

In one embodiment, said 4-1 BB-specific recombinant binding protein of the invention further comprises a polypeptide tag. A polypeptide tag is an amino acid sequence attached to a polypeptide/protein, wherein said amino acid sequence is useful for the purification, detection, or targeting of said polypeptide/protein, or wherein said amino acid sequence improves the physicochemical behavior of the polypeptide/protein, or wherein said amino acid sequence possesses an effector function. The individual polypeptide tags of a binding protein may be connected to other parts of the binding protein directly or via peptide linkers. Polypeptide tags are all well known in the art and are fully available to the person skilled in the art. Examples of polypeptide tags are small polypeptide sequences, for example, His, HA, myc, FLAG, or Strep-tags, or polypeptides such as enzymes (for example alkaline phosphatase), which allow the detection of said polypeptide/protein, or polypeptides which can be used for targeting (such as immunoglobulins or fragments thereof) and/or as effector molecules.

In one embodiment, said 4-1 BB-specific recombinant binding protein of the invention further comprises a peptide linker. A peptide linker is an amino acid sequence, which is able to link, for example, two protein domains, a polypeptide tag and a protein domain, a protein domain and a non-proteinaceous compound or polymer such as polyethylene glycol, a protein domain and a biologically active molecule, a protein domain and a localizer, or two sequence tags. Peptide linkers are known to the person skilled in the art. A list of examples is provided in the description of patent application WO2002/020565. Particular examples of such linkers are glycine-serine-linkers and proline-threonine-linkers of variable lengths. Examples of a glycine-serine-linker are the amino acid sequence GS and the amino acid sequence of SEQ ID NO:40, and examples of a proline-threonine-linker are the amino acid sequences of SEQ ID NO:39 and SEQ ID NO:57.

In another aspect, the invention relates to a nucleic acid encoding the amino acid sequence of an ankyrin repeat domain or a recombinant binding protein of the present invention. In one embodiment, the invention relates to a nucleic acid encoding the amino acid sequence of a recombinant binding protein of the present invention. In one embodiment, the invention relates to a nucleic acid encoding an amino acid sequence selected from the group consisting of SEQ ID NO: 1 to 38. In one embodiment, the invention relates to a nucleic acid encoding an amino acid sequence selected from the group consisting of SEQ ID NO: 1 to 27. In one embodiment, the invention relates to a nucleic acid encoding an amino acid sequence selected from the group consisting of SEQ ID NO: 1, SEQ ID NO: 5, SEQ ID NO: 31, SEQ ID NO: 51, and SEQ ID NO: 54. In one embodiment, the invention relates to a nucleic acid encoding the amino acid sequence of SEQ ID NO: 1. In one embodiment, the invention relates to a nucleic acid encoding the amino acid sequence of SEQ ID NO: 5. In one embodiment, the invention relates to a nucleic acid encoding the amino acid sequence of SEQ ID NO: 31. In one embodiment, the invention relates to a nucleic acid encoding the amino acid sequence of SEQ ID NO: 51. In one embodiment, the invention relates to a nucleic acid encoding the amino acid sequence of SEQ ID NO: 54. Furthermore, the invention relates to vectors comprising any nucleic acid of the invention. Nucleic acids are well known to the skilled person in the art. In the examples, nucleic acids were used to produce designed ankyrin repeat domains or recombinant binding proteins of the invention in E. coli. An example of a nucleic acid of the invention is provided by SEQ ID NO: 41, which encodes the amino acid sequence of SEQ ID NO: 1.

In one aspect, the invention relates to a pharmaceutical composition comprising a recombinant binding protein and/or a designed ankyrin repeat domain of the present invention, and/or a nucleic acid encoding a recombinant binding protein and/or a designed ankyrin repeat domain of the present invention, and optionally a pharmaceutically acceptable carrier and/or diluent.

In one embodiment, the invention relates to a pharmaceutical composition comprising a recombinant binding protein or a nucleic acid encoding a recombinant binding protein of the present invention, and optionally a pharmaceutically acceptable carrier and/or diluent.

Pharmaceutically acceptable carriers and/or diluents are known to the person skilled in the art and are explained in more detail below. Even further, a diagnostic composition is provided comprising one or more of the above mentioned recombinant binding proteins and/or designed ankyrin repeat domains, and/or nucleic acids, in particular recombinant binding proteins and/or nucleic acids of the present invention.

A pharmaceutical composition comprises a recombinant binding protein, and/or a designed ankyrin repeat domain, and/or a nucleic acid, preferably a recombinant binding protein and/or a nucleic acid, as described herein and a pharmaceutically acceptable carrier, excipient or stabilizer, for example as described in Remington's Pharmaceutical Sciences 16th edition, Osol, A. Ed., 1980.

Suitable carriers, excipients or stabilizers known to one of skill in the art include, for example, saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polylactic acids, polyglycolic acids, polymeric amino acids and amino acid copolymers. A pharmaceutical composition may also be a combination formulation, comprising an additional active agent, such as an anti-cancer agent or an anti-angiogenic agent, or an additional bioactive compound.

The formulations to be used for in vivo administration must be aseptic or sterile. This is readily accomplished by filtration through sterile filtration membranes.

One embodiment of the present invention relates to the use of a recombinant binding protein of the present invention comprising an ankyrin repeat domain having binding specificity for 4-1 BB and further comprising an ankyrin repeat domain with binding specificity for serum albumin for manufacturing a pharmaceutical composition, wherein said recombinant binding protein exhibits an increased terminal half-life, preferably an increased terminal half-life of at least 5%, preferably 10%, 15%, 20%, 25%, 30%, 35%, 40%, 45%, 50%, 60%, 70%, 80%, 90%, 100%, 150%, 200%, or 250%, compared to a corresponding recombinant binding protein comprising said ankyrin repeat domain with binding specificity for 4-1 BB but not said ankyrin repeat domain with binding specificity for serum albumin. In one embodiment of the invention, a recombinant binding protein comprises an ankyrin repeat domain having binding specificity for 4-1BB and further comprises two ankyrin repeat domains with binding specificity for serum albumin.

In one embodiment, a pharmaceutical composition comprises at least one recombinant binding protein as described herein and a detergent such as nonionic detergent, a buffer such as phosphate buffer, and a sugar such as sucrose. In one embodiment, such a composition comprises recombinant binding proteins as described above and PBS.

In another aspect, the invention provides a method of localized activation of 4-1 BB in 4-1 BB expressing cells or tissue in a mammal, the method comprising the step of administering to said mammal the inventive recombinant binding protein comprising an ankyrin repeat domain with binding specificity for 4-1 BB and further comprising a localizer molecule. In one embodiment, said localizer molecule is a binding protein having binding specificity for a target different from 4-1 BB. In one embodiment, said mammal is a human and said 4-1 BB-expressing cells or tissue are located in a tumor, including in a primary tumor, metastasis and/or tumor stroma.

In another aspect, the invention provides a method of treating a medical condition, the method comprising the step of administering to a patient in need thereof a therapeutically effective amount of the inventive recombinant binding protein comprising a localizer molecule. In another aspect, the invention provides a method of treating a medical condition, the method comprising the step of administering to a patient in need thereof a therapeutically effective amount of the inventive recombinant binding protein comprising a localizer molecule, wherein said localizer molecule is effective in localizing said binding protein to target tissue, and wherein said localization of said binding protein results in activation of 4-1BB in 4-1BB-expressing cells in the target tissue. In one embodiment, said localizer molecule is a binding protein having binding specificity for a cell surface protein expressed in a tumor, wherein said cell surface protein is different from 4-1 BB. In one embodiment, said 4-1 BB-expressing cells or tissue are located in a tumor, including in a primary tumor, metastasis and/or tumor stroma. These embodiments thus allow to take advantage of the localizer's restricted expression in a tumor by localizing the activation of 4-1 BB by the recombinant binding protein of the invention to the tumor.

In another aspect, the invention provides a method of diagnosing a medical condition in a patient, the method comprising the step of administering the inventive recombinant binding protein comprising an ankyrin repeat domain with binding specificity for human 4-1 BB to a patient in need of said diagnosis or to a body fluid or tissue sample of a patient in need of said diagnosis. In one embodiment, said body fluid is blood plasma or a derivative thereof. In one embodiment, said body fluid is blood serum. In one embodiment, said tissue is tumor tissue. In one embodiment, said medical condition is cancer. In another embodiment, said medical condition is an autoimmune disease.

In one embodiment, the invention relates to the use of a pharmaceutical composition, or a recombinant binding protein according to the present invention for the treatment of a disease. For that purpose, the pharmaceutical composition, or the recombinant binding protein according to the present invention is administered, to a patient in need thereof, in a therapeutically effective amount. Administration may include topical administration, oral administration, and parenteral administration. The typical route of administration is parenteral administration. In parental administration, the pharmaceutical composition of this invention will be formulated in a unit dosage injectable form such as a solution, suspension or emulsion, in association with the pharmaceutically acceptable excipients as defined above. The dosage and mode of administration will depend on the individual to be treated and the particular disease.

Further, any of the above mentioned pharmaceutical composition or recombinant binding protein is considered for the treatment of a disorder.

In one embodiment, said recombinant binding protein or such other pharmaceutical composition described herein is applied intravenously. For parenteral application, the recombinant binding protein or said pharmaceutical composition can be injected as bolus injection or by slow infusion at a therapeutically effective amount.

In one embodiment, the invention relates to a method of treatment of a medical condition, the method comprising the step of administering, to a patient in need of such a treatment, a therapeutically effective amount of a recombinant binding protein of the invention. In one embodiment, the invention relates to a method of treatment of a medical condition, the method comprising the step of administering, to a patient in need of such a treatment, a therapeutically effective amount of a pharmaceutical composition of the invention. In one embodiment, the invention relates to the use of a pharmaceutical composition of the present invention for the treatment of a disease. In one embodiment, the invention relates to a pharmaceutical composition for use in the treatment of a disease. In one embodiment, the invention relates to a pharmaceutical composition for use in the treatment of a medical condition. In one embodiment, the invention relates to a nucleic acid for use in the treatment of a disease. In one embodiment, the invention relates to the use of said pharmaceutical composition, recombinant binding protein, or nucleic acid molecule, as medicament for the treatment of a disease. In one embodiment, the invention relates to the use of said pharmaceutical composition, recombinant binding protein, or nucleic acid molecule, for manufacturing of a medicament. In one embodiment, the invention relates to the use of said pharmaceutical composition, recombinant binding protein, or nucleic acid molecule, for manufacturing of a medicament for the treatment of a disease. In one embodiment, the invention relates to a process for the manufacturing of a medicament for the treatment of a disease, wherein said pharmaceutical composition, recombinant binding protein, or nucleic acid molecule is active ingredient of the medicament. In one embodiment, the invention relates to a process of treatment of a disease using said pharmaceutical composition, recombinant binding protein, or nucleic acid molecule.

In particular, the invention relates to the treatment of a medical condition using a pharmaceutical composition of the present invention, wherein said medical condition is cancer.

The use of a recombinant binding protein of the present invention or said pharmaceutical compositions for the treatment of cancer diseases can also be in combination with one or more other therapies known in the art. The term “use in combination with”, as used herein, shall refer to a co-administration, which is carried out under a given regimen. This includes synchronous administration of the different compounds as well as time-shifted administration of the different compounds (e.g. compound A is given once and compound B is given several times thereafter, or vice versa, or both compounds are given synchronously and one of the two is also given at later stages).

In a further embodiment, the invention relates to the use of a recombinant binding protein of the invention for the manufacture of a medicament that is used for the treatment of a medical condition, preferably a neoplastic disease, more preferably cancer.

In one embodiment, the invention relates to the use of a pharmaceutical composition of the invention for the manufacture of a medicament that is used for the treatment of a medical condition, which may be a neoplastic disease, in particular cancer.

In one embodiment the invention relates to a recombinant binding protein comprising any of the above mentioned ankyrin repeat domains.

In one embodiment, the invention relates to a kit comprising said recombinant binding protein. In one embodiment, the invention relates to a kit comprising a nucleic acid encoding said recombinant binding protein. In one embodiment, the invention relates to a kit comprising said pharmaceutical composition. In one embodiment, the invention relates to a kit comprising said recombinant binding protein, and/or a nucleic acid encoding said recombinant binding protein, and/or said pharmaceutical composition. In one embodiment, the invention relates to a kit comprising the recombinant binding protein comprising a 4-1 BB-specific ankyrin repeat domain, for example SEQ ID NO:1 or SEQ ID NO: 5, and/or a nucleic acid encoding the recombinant binding protein comprising a 4-1 BB-specific ankyrin repeat domain, for example SEQ ID NO:1 or SEQ ID NO: 5, and/or a pharmaceutical composition comprising the recombinant binding protein comprising a 4-1 BB-specific ankyrin repeat domain, for example SEQ ID NO:1 or SEQ ID NO: 5, and/or a nucleic acid encoding the recombinant binding protein comprising a 4-1 BB-specific ankyrin repeat domain, for example SEQ ID NO:1 or SEQ ID NO: 5.

In one embodiment, the invention relates to a method for producing a recombinant binding protein of the present invention. In one embodiment, the invention relates to a method for producing a recombinant binding protein, for example a recombinant binding protein comprising the amino acid sequence of SEQ ID NO:1 or SEQ ID NO:5, the method comprising the steps of (i) expressing said recombinant binding protein in bacteria, and (ii) purifying said recombinant binding protein using chromatography. Said method may comprise additional steps. Such a method of producing a recombinant binding protein of the present invention is described in Example 1.

The invention is not restricted to the particular embodiments described in the Examples.

This specification refers to a number of amino acid sequences of the amino acid sequence listing of this specification named “P5725_Sequence_Listing.txt” and the amino acid sequences of the sequence listing are herewith incorporated by reference.

### DEFINITIONS

Unless defined otherwise herein, all technical and scientific terms used herein shall have the meanings that are commonly understood by those of ordinary skill in the art to which the present invention belongs.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g. by a disulfide bond between two polypeptides. A part of a protein, which individually has, or is able to acquire, a defined three-dimensional arrangement by forming secondary and/or tertiary structure, is termed “protein domain”. Such protein domains are well known to the practitioner skilled in the art.

The term “recombinant” as used in recombinant protein, recombinant polypeptide and the like, means that said protein or polypeptide is produced by the use of recombinant DNA technologies well known to the practitioner skilled in the art. For example, a recombinant DNA molecule (e.g. produced by gene synthesis) encoding a polypeptide can be cloned into a bacterial expression plasmid (e.g. pQE30, QIAgen), yeast expression plasmid, mammalian expression plasmid, or plant expression plasmid, or a DNA enabling in vitro expression. If, for example, such a recombinant bacterial expression plasmid is inserted into appropriate bacteria (e.g. Escherichia coli), these bacteria can produce the polypeptide(s) encoded by this recombinant DNA. The correspondingly produced polypeptide or protein is called a recombinant polypeptide or recombinant protein.

In the context of the present invention, the term “binding protein” refers to a protein comprising a binding domain. A binding protein may also comprise two, three, four, five or more binding domains. Preferably, said binding protein is a recombinant binding protein. Binding proteins of the instant invention comprise an ankyrin repeat domain with binding specificity for 4-1 BB.

Furthermore, any such binding protein may comprise additional polypeptides (such as e.g. polypeptide tags, peptide linkers, fusion to other proteinaceous domains with binding specificity, cytokines, hormones, or antagonists), or chemical modifications (such as coupling to polyethylene-glycol, toxins (e.g. DM1 from Immunogen), small molecules, antibiotics and alike) well known to the person skilled in the art. A binding protein of the instant invention may comprise a localizer molecule.

The term “binding domain” means a protein domain exhibiting binding specificity for a target. Preferably, said binding domain is a recombinant binding domain.

The term “target” refers to an individual molecule such as a nucleic acid molecule, a polypeptide or protein, a carbohydrate, or any other naturally occurring molecule, including any part of such individual molecule, or to complexes of two or more of such molecules, or to a whole cell or a tissue sample, or to any non-natural compound. Preferably, a target is a naturally occurring or non-natural polypeptide or protein, or a polypeptide or protein containing chemical modifications, for example, naturally occurring or non-natural phosphorylation, acetylation, or methylation. In the context of the present invention, 4-1 BB and 4-1BB-expressing cells and tissues are targets of 4-1 BB-specific binding proteins and localizer target proteins and cells and tissues are targets of localizers.

In the context of the present invention, the term “polypeptide” relates to a molecule consisting of a chain of multiple, i.e. two or more, amino acids linked via peptide bonds. Preferably, a polypeptide consists of more than eight amino acids linked via peptide bonds. The term “polypeptide” also includes multiple chains of amino acids, linked together by S-S bridges of cysteines. Polypeptides are well-known to the person skilled in the art.

Patent application WO2002/020565 and Forrer et al., 2003 (Forrer, P., Stumpp, M. T., Binz, H. K., Plückthun, A., 2003. FEBS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers. The repeat domains can be binding domains.

The term “repeat domain” refers to a protein domain comprising two or more consecutive repeat modules as structural units, wherein said repeat modules have structural and sequence homology. Preferably, a repeat domain further comprises an N-terminal and/or a C-terminal capping module. For clarity, a capping module can be a repeat module. Such repeat domains, repeat modules, and capping modules, sequence motives, as well as structural homology and sequence homology are well known to the practitioner in the art from examples of ankyrin repeat domains (WO2002/020565), leucine-rich repeat domains (WO2002/020565), tetratricopeptide repeat domains (Main, E. R., Xiong, Y., Cocco, M. J., D'Andrea, L., Regan, L., Structure 11(5), 497-508, 2003), and armadillo repeat domains (WO2009/040338). It is further well known to the practitioner in the art, that such repeat domains are different from proteins comprising repeated amino acid sequences, where every repeated amino acid sequence is able to form an individual domain (for example FN3 domains of Fibronectin).

The term “designed” as used in designed repeat protein, designed repeat domain and the like refers to the property that such repeat proteins and repeat domains, respectively, are man-made and do not occur in nature. The binding proteins of the instant invention are designed repeat proteins and they comprise at least one designed ankyrin repeat domain.

The term “target interaction residues” refers to amino acid residues of a repeat module, which contribute to the direct interaction with a target.

The term “framework residues” refers to amino acid residues of a repeat module, which contribute to the folding topology, i.e. which contribute to the fold of said repeat module or which contribute to the interaction with a neighboring module. Such contribution may be the interaction with other residues in the repeat module, or the influence on the polypeptide backbone conformation as found in α-helices or β-sheets, or the participation in amino acid stretches forming linear polypeptides or loops.

Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

The term “repeat modules” refers to the repeated amino acid sequence and structural units of the designed repeat domains, which are originally derived from the repeat units of naturally occurring repeat proteins. Each repeat module comprised in a repeat domain is derived from one or more repeat units of a family or subfamily of naturally occurring repeat proteins, e.g. the family of ankyrin repeat proteins. Furthermore, each repeat module comprised in a repeat domain may comprise a “repeat sequence motif” deduced from homologous repeat modules obtained from repeat domains selected on a target, e.g. as described in Example 1, and having the same target specificity.

Accordingly, the term “ankyrin repeat module” refers to a repeat module, which is originally derived from the repeat units of naturally occurring ankyrin repeat proteins. Ankyrin repeat proteins are well known to the person skilled in the art.

Repeat modules may comprise positions with amino acid residues which have not been randomized in a library for the purpose of selecting target-specific repeat domains (“non-randomized positions”) and positions with amino acid residues which have been randomized in the library for the purpose of selecting target-specific repeat domains (“randomized positions”). The non-randomized positions comprise framework residues. The randomized positions comprise target interaction residues. “Have been randomized” means that two or more amino acids were allowed at an amino acid position of a repeat module, for example, wherein any of the usual twenty naturally occurring amino acids were allowed, or wherein most of the twenty naturally occurring amino acids were allowed, such as amino acids other than cysteine, or amino acids other than glycine, cysteine and proline. For the purpose of this patent application, amino acid residues 3, 4, 6, 14 and 15 of SEQ ID NOs: 58 to 66 and 68 to 71 and amino acid residues 3, 5, 7, 15 and 16 of SEQ ID NO: 67 are randomized positions of the ankyrin repeat modules of the instant invention.

The term “repeat sequence motif” refers to an amino acid sequence, which is deduced from one or more repeat modules. Preferably, said repeat modules are from repeat domains having binding specificity for the same target. Such repeat sequence motifs comprise framework residue positions and target interaction residue positions. Said framework residue positions correspond to the positions of framework residues of the repeat modules. Likewise, said target interaction residue positions correspond to the positions of target interaction residues of the repeat modules. Repeat sequence motifs comprise non-randomized positions and randomized positions.

The term “repeat unit” refers to amino acid sequences comprising sequence motifs of one or more naturally occurring proteins, wherein said “repeat units” are found in multiple copies, and exhibit a defined folding topology common to all said motifs determining the fold of the protein. Examples of such repeat units include leucine-rich repeat units, ankyrin repeat units, armadillo repeat units, tetratricopeptide repeat units, HEAT repeat units, and leucine-rich variant repeat units.

The term “has binding specificity for a target”, “specifically binding to a target”, “binding to a target with high specificity”, “specific for a target” or “target specificity” and the like means that a binding protein or binding domain binds in PBS to a target with a lower dissociation constant (i.e. it binds with higher affinity) than it binds to an unrelated protein such as the E. coli maltose binding protein (MBP). Preferably, the dissociation constant (“K_{D}”) in PBS for the target is at least 10²; more preferably, at least 10³; more preferably, at least 10⁴; or more preferably, at least 10⁵ times lower than the corresponding dissociation constant for MBP. Methods to determine dissociation constants of protein-protein interactions, such as surface plasmon resonance (SPR) based technologies (e.g. SPR equilibrium analysis) or isothermal titration calorimetry (ITC) are well known to the person skilled in the art. The measured K_{D} values of a particular protein-protein interaction can vary if measured under different conditions (e.g., salt concentration, pH). Thus, measurements of K_{D} values are preferably made with standardized solutions of protein and a standardized buffer, such as PBS. A typical and preferred determination of dissociation constants (K_{D}) of the inventive recombinant binding proteins with binding specificity for 4-1BB by Surface Plasmon Resonance (SPR) analysis is described in Example 2.

The term “about” means the mentioned value +/−20%; for example “about 50” shall mean 40 to 60.

The term “PBS” means a phosphate buffered water solution containing 137 mM NaCl, 10 mM phosphate and 2.7 mM KCI and having a pH of 7.4.

The term “mouse serum albumin” refers to UniProt accession number P07724, the term “cynomolgus monkey serum albumin” (i.e. macaca fascicularis) refers to UniProt accession number A2V9Z4, and the term “human serum albumin” refers to UniProt accession number P02768.

Preferably, clearance, and/or exposure, and/or terminal half-life are assessed in a mammal, more preferably mouse and/or cynomolgus monkey, more preferably cynomolgus monkey. Preferably, when measuring the clearance, and/or exposure, and/or terminal half-life in mouse, the evaluation is done considering the data up to 48 h post-injection. More preferably, the evaluation of terminal half-life in mouse is calculated from 24 h to 48 h. Preferably, when measuring the clearance, and/or exposure, and/or terminal half-life in cynomolgus monkey, the evaluation is done considering the data up to day 7 post-injection. More preferably, the evaluation of terminal half-life in cynomolgus monkey is calculated from day 1 to day 5. The person skilled in the art further is able to identify effects such as target-mediated clearance and consider them when calculating the terminal half-life. The term “terminal half-life” of a drug such as a recombinant binding protein of the invention refers to the time required to reach half the plasma concentration of the drug applied to a mammal after reaching pseudo-equilibrium (for example calculated from 24 hours to 48 hours in mouse or calculated from day 1 to day 5 in cynomolgus monkey). Terminal half-life is not defined as the time required to eliminate half the dose of the drug administered to the mammal. The term terminal half-life is well known to the person skilled in the art. Preferably, pharmacokinetic comparison is done at any dose, more preferably at equivalent dose (i.e. same mg/kg dose) or equimolar dose (i.e. same mol/kg dose), more preferably at equimolar dose (i.e. same mol/kg dose). It is understood by the person skilled in the art that equivalent and/or equimolar dosing in animals is subject to experimental dose variations of at least 20%, more preferably 30%, 40%, 50%, 60%, 70%, 80%, 90%, or 100%. Preferably, a dose used for pharmacokinetic measurement is selected from 0.001 to 1000 mg/kg, more preferably 0.01 to 100 mg/kg, more preferably 0.1 to 50 mg/kg, more preferably 0.5 to 10 mg/kg.

The terms “4-1 BB” and “4-1 BB receptor” are used interchangeably in the present application, and refer to any form of 4-1 BB receptor, as well as to variants, isoforms, and species homologs thereof that retain at least a part of the activity of 4-1 BB receptor. Accordingly, a binding protein, as defined and disclosed herein, may also bind 4-1 BB from species other than human. In other cases, a binding protein may be completely specific for the human 4-1 BB and may not exhibit species or other types of cross-reactivity. Unless indicated differently, such as by specific reference to human 4-1 BB, 4-1 BB includes all mammalian species of native sequence 4-1BB, e.g., human, canine, feline, equine and bovine. An amino acid sequence of human 4-1BB is shown in NCBI (www.ncbi.nlm.nih.gov/) Ref. Seq. NP_001552 and in SEQ ID NO: 76. Amino acid sequences of cynomolgus and mouse 4-1 BB are provided in SEQ ID NOs: 77 and 78, respectively. 4-1 BB comprises a signal sequence, followed by an extracellular domain, a transmembrane region, and an intracellular domain (Cheuk ATC et al. 2004 Cancer Gene Therapy 11: 215-226). The receptor is thought to undergo receptor trimerization upon binding to its trimeric ligand (4-1BBL) to stimulate immune responses (Bitra A et al, J. Biol. Chem. 2018, 293(4):1317-1329; Chin et al, Nature Communications (2018) 9:4679). Alternative names or synonyms for 4-1BB include CD137 and TNFRSF9. The amino acid sequences of the extracellular domains of human, cynomolgus and mouse 4-1BB are shown in SEQ ID NOs: 79, 80 and 81, respectively. An amino acid sequence of human 4-1 BBL is shown in NCBI Ref. Seq. NP_003802.1.

“4-1 BB agonist” as used herein means any chemical compound or biological molecule, which upon binding to 4-1BB, (1) stimulates or activates 4-1BB, (2) enhances, increases, promotes, induces, or prolongs an activity, function, or presence of 4-1 BB, or (3) enhances, increases, promotes, or induces the expression of 4-1 BB. In any of the treatment methods, medicaments, pharmaceutical compositions and uses of the present invention in which a human individual is being treated, the 4-1BB agonists increase a 4-1BB-mediated response. In some embodiments of the treatment methods, medicaments, pharmaceutical compositions and uses of the present invention, 4-1BB agonists markedly enhance cytotoxic T-cell responses, resulting in anti-tumor activity in various models.

The term “localizing” or “delivering” as interchangeably used herein in the context of a “localizer molecule” or “localizer” comprised by a 4-1 BB-specific binding protein of the invention refers to increased localization of such 4-1BB-specific binding protein comprising a localizer, as compared to when the binding protein does not comprise the localizer, to localizer target cells or tissue in a mammal. The term also refers to targeting a 4-1 BB-specific binding protein to the site of localizer target cells or tissue in a mammal, wherein the 4-1 BB-specific binding protein comprises the localizer. The term preferably further encompasses the accumulation and/or retention of a 4-1BB-specific binding protein comprising a localizer at the site of localizer target cells or tissue in a mammal. The term also preferably encompasses the localized activation of 4-1 BB in 4-1 BB-expressing cells induced by a 4-1 BB-specific binding protein of the invention comprising a localizer at or nearby the site of localizer target cells or tissue in a mammal. Such localized activation may occur, for example, through clustering of 4-1 BB upon binding of the 4-1 BB-specific binding protein comprising the localizer, wherein the clustering is mediated by binding of the localizer to its target cells or tissue. As used in this paragraph, “mammal” encompasses human. The result of “localizing” may be measured by various means well known to one of skill in the art. As an example, “localizing” may be measured by determining the organ-to-blood ratio of a binding protein of the invention linked to a localizer, according to methods well known in the art. In one embodiment of the invention, the effect of a localizer on “localizing” a 4-1 BB-specific binding protein comprising a localizer is exhibited by an increased organ-to-blood ratio of at least 5%, 10%, 15%, 20%, 25%, 30%, 35%, 40%, 45%, 50%, 60%, 70%, 80%, 90%, 100%, 150%, 200%, 250%, or 300% compared to a corresponding 4-1BB-specific binding protein that does not comprise the localizer.

The terms “localizer molecule” and “localizer” are used interchangeably herein and are intended to encompass any molecule that can be comprised by a 4-1 BB-specific binding protein of the invention and that is capable of localizing or delivering such a 4-1 BB-specific binding protein of the invention comprising it to target cells or tissue in a mammal, including, e.g., a human, wherein the localizer binds to the target cells or tissue. A localizer can be linked, conjugated, fused or otherwise physically attached to a 4-1 BB-specific ankyrin repeat domain of the invention. The terms “localizer molecule” and “localizer” encompass polynucleotides, peptides/polypeptides and/or chemical or biochemical agents that have these properties. The term “polynucleotides” generally refers to DNA or RNA, and includes modified and artificial forms of DNA or RNA. The term “peptide” refers to a peptide chain of 4 to 600 amino acids long, such as 4 to 200 amino acids long, and therefore encompasses polypeptides and proteins. The term encompasses any naturally occurring or man-made binding proteins, binding domains, growth factor receptors or fragments or ligands thereof, cytokines, polypeptide hormones, antibodies, antibody-like proteins based on scaffolds, immunomodulatory proteins, etc. Furthermore, the term “peptide” also encompasses peptides modified by, e.g, glycosylation, and proteins comprising two or more polypeptide chains, each of length of 4 to 600 amino acids long, cross-linked by, e.g., disulphide bonds, such as, e.g., insulin and immunoglobulins. The term “chemical or biochemical agent” is intended to include any naturally occurring or synthetic compound that may be administered to a recipient. In a preferred embodiment, the localizer is a target-specific ankyrin repeat domain.

The term “4-1 BB-expressing cells” as used herein refers to any cells expressing 4-1 BB on the cell surface, including, but not limited, to T-cells such as CD8⁺ T cells, regulatory T cells (Treg) and NK T cells (NKT), neutrophils, granulocytes, monocytes, mast cells, eosinophils, dendritic cells (DC), natural killer (NK) cells, B-lymphocytes and leucocytes, as well as cells of non- hematopoietic origin such as endothelial cells and smooth muscle cells.

The term “medical condition” (or disorder or disease) includes autoimmune disorders, inflammatory disorders, retinopathies (particularly proliferative retinopathies), neurodegenerative disorders, infections, metabolic diseases, and neoplastic diseases. Any of the recombinant binding proteins described herein may be used for the preparation of a medicament for the treatment of such a disorder, particularly a disorder selected from the group comprising: an autoimmune disorder, an inflammatory disorder, an immune disorder, and a neoplastic disease. A “medical condition” may be one that is characterized by inappropriate cell proliferation. A medical condition may be a hyperproliferative condition. The invention particularly relates to a method of treating a medical condition, the method comprising the step of administering, to a patient in need of such treatment, a therapeutically effective amount of a recombinant binding protein or said pharmaceutical composition of the invention. In a preferred embodiment said medical condition is a neoplastic disease. The term “neoplastic disease”, as used herein, refers to an abnormal state or condition of cells or tissue characterized by rapidly proliferating cell growth or neoplasm. In one embodiment said medical condition is a malignant neoplastic disease. In one embodiment said medical condition is a cancer. The term “therapeutically effective amount” means an amount that is sufficient to produce a desired effect on a patient.

The term “antibody” means not only intact antibody molecules, but also any fragments and variants of antibody molecules that retain immunogen-binding ability. Such fragments and variants are also well known in the art and are regularly employed both in vitro and in vivo. Accordingly, the term “antibody” encompasses intact immunoglobulin molecules, antibody fragments such as, e.g., Fab, Fab′, F(ab′)₂, and single chain V region fragments (scFv), bispecific antibodies, chimeric antibodies, antibody fusion polypeptides, and unconventional antibodies.

The terms “cancer” and “cancerous” are used herein to refer to or describe the physiological condition in mammals that is typically characterized by unregulated cell growth. Cancer encompasses solid tumors and liquid tumors, as well as primary tumors and metastases. A “tumor” comprises one or more cancerous cells. Solid tumors typically also comprise tumor stroma. Examples of cancer include, but are not limited to, primary and metastatic carcinoma, lymphoma, blastoma, sarcoma, and leukemia, and any other epithelial and lymphoid malignancies. More particular examples of such cancers include brain cancer, bladder cancer, breast cancer, ovarian cancer, clear cell kidney cancer, head/neck squamous cell carcinoma, lung adenocarcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), Squamous Cell Carcinoma of the Head and Neck (SCCHN), chronic myelogenous leukemia (CML), small lymphocytic lymphoma (SLL), malignant mesothelioma, colorectal cancer, or gastric cancer.

### EXAMPLES

Starting materials and reagents disclosed below are known to those skilled in the art, are commercially available and/or can be prepared using well-known techniques.

### Materials

Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). Inducible E. coli expression strains were used for cloning and protein production, e.g. E. co/iXL1-blue (Stratagene, USA) or BL21 (Novagen, USA). Recombinant Fc fusion proteins of the extracellular domain of human 4-1BB (biotinylated or non-biotinylated) were purchased from BPS Bioscience. A recombinant Fc fusion protein of the extracellular domain of cynomolgus 4-1 BB was purchased from Evitria AG.

### Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989, New York).

### Designed Ankyrin Repeat Protein Libraries

Methods to generate designed ankyrin repeat protein libraries have been described, e.g. in U.S. Pat. No. 7,417,130; Binz et al. 2003, loc. cit.; Binz et al. 2004, loc. cit. By such methods designed ankyrin repeat protein libraries having randomized ankyrin repeat modules and/or randomized capping modules can be constructed. For example, such libraries could accordingly be assembled based on a fixed N-terminal capping module (e.g. the N-terminal capping module of SEQ ID NO: 44, 45 or 46) or a randomized N-terminal capping module according to SEQ ID NO: 47, one or more randomized repeat modules according to the sequence motif of SEQ ID NO: 48, 49 or 50, and a fixed C-terminal capping module (e.g. the C-terminal capping module of SEQ ID NO: 72, 73 or 74) or a randomized C-terminal capping module according to SEQ ID NO: 75. Preferably, such libraries are assembled to not have any of the amino acids C, G, M, N (in front of a G residue) and P at randomized positions of repeat or capping modules. In addition, randomized repeat modules according to the sequence motif of SEQ ID NO: 48, 49 or 50 could be further randomized at position 10 and/or position 17; the randomized N-terminal capping module according to the sequence motif of SEQ ID NO: 47 could be further randomized at position 7 and/or position 9; and the randomized C-terminal capping modules according to the sequence motif of SEQ ID NO: 75 could be further randomized at positions 10, 11 and/or 17.

Furthermore, such randomized modules in such libraries may comprise additional polypeptide loop insertions with randomized amino acid positions. Examples of such polypeptide loop insertions are complement determining region (CDR) loop libraries of antibodies or de novo generated peptide libraries. For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23(30), 3929-3938, 2004) as guidance. In analogy to this ankyrin repeat domain where ten amino acids are inserted in the beta-turn present close to the boarder of two ankyrin repeats, ankyrin repeat proteins libraries may contain randomized loops (with fixed and randomized positions) of variable length (e.g. 1 to 20 amino acids) inserted in one or more beta-turns of an ankyrin repeat domain.

Any such N-terminal capping module of an ankyrin repeat protein library preferably possesses the RILLAA, RILLKA or RELLKA motif (e.g. present from position 21 to 26 in SEQ ID NO: 1) and any such C-terminal capping module of an ankyrin repeat protein library preferably possesses the KLN, KLA or KAA motif (e.g. present at the last three amino acids in SEQ ID NO: 1).

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V2O, 3UXG, 3TWQ-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

The nomenclature used to define the positions inside the repeat units and modules is based on Binz et al. 2004, loc. cit. with the modification that borders of the ankyrin repeat modules and ankyrin repeat units are shifted by one amino acid position. For example, position 1 of an ankyrin repeat module of Binz et al. 2004 (loc. cit.) corresponds to position 2 of an ankyrin repeat module of the current disclosure and consequently position 33 of an ankyrin repeat module of Binz et al. 2004, loc. cit. corresponds to position 1 of a following ankyrin repeat module of the current disclosure.

All the DNA sequences were confirmed by sequencing, and the calculated molecular weight of selected proteins was confirmed by mass spectrometry.

### Example 1: Selection of Binding Proteins Comprising an Ankyrin Repeat Domain with Binding Specificity for 4-1BB

Using ribosome display (Hanes, J. and Plückthun, A., PNAS 94, 4937-42, 1997), many ankyrin repeat proteins with binding specificity for human 4-1 BB (h4-1 BB) were selected from DARPin® libraries similar as described by Binz et al. 2004 (loc. cit.). The binding of the selected clones toward recombinant human 4-1BB target was assessed by crude extract Homogeneous Time Resolved Fluorescence (HTRF), indicating that hundreds of h4-1 BB-specific binding proteins were successfully selected. For example, the ankyrin repeat domains of SEQ ID NO: 1 to 27 constitute amino acid sequences of selected binding proteins comprising an ankyrin repeat domain with binding specificity for h4-1 BB. Individual ankyrin repeat modules from such ankyrin repeat domains with binding specificity to h4-1 BB are provided, e.g., in SEQ ID NO: 58 to 71.

Selection of 4-18B-Specific Ankyrin Repeat Proteins by Ribosome Display

The selection of h4-1 BB-specific ankyrin repeat proteins was performed by ribosome display (Hanes and Plückthun, loc. cit.) using the extracellular domain of human 4-1 BB (SEQ ID NO: 79) (alone or in a complex with h4-1 BBL) as target protein, libraries of ankyrin repeat proteins as described above, and established protocols (See, e.g., Zahnd, C., Amstutz, P. and Plückthun, A., Nat. Methods 4, 69-79, 2007). The number of reverse transcription (RT)-PCR cycles after each selection round was constantly reduced from 45 to 28, adjusting to the yield due to enrichment of binders. The first four rounds of selection employed standard ribosome display selection, using decreasing target concentration and increasing washing stringency to increase selection pressure from round 1 to round 4 (Binz et al. 2004, loc. cit.). For some pools, low salt concentrations were used during washing steps in all ribosome display selection rounds. To enrich high affinity 4-1 BB-specific ankyrin repeat proteins, the output from the fourth round of standard ribosome display selection (above) was subjected to an off-rate selection round with increased selection stringency (Zahnd, 2007, loc. cit.). A final standard selection round was performed after the off-rate selection round to amplify and recover the off-rate selected binding proteins. In these last two selection rounds, the number of RT-PCR cycles was kept constant at 30.

Selected Clones Bind Specifically to 4-1BB as Shown by Crude Extract HTRF

Individual selected ankyrin repeat proteins specifically binding 4-1 BB in solution were identified by a Homogeneous Time Resolved Fluorescence (HTRF) assay using crude extracts of ankyrin repeat protein-expressing Escherichia coli cells using standard protocols. Ankyrin repeat protein clones selected by ribosome display were cloned into derivatives of the pQE30 (Qiagen) expression vector (either pMPAG06, pMPAS242 or pMPAS245), transformed into E. coli XL1-Blue (Stratagene), plated on LB-agar (containing 1% glucose and 50 μg/ml ampicillin) and then incubated overnight at 37° C. Vectors pMPAS242 or pMPAS245 were used to exchange N- and C-terminal capping repeats. Single colonies were picked into a 96 well plate (each clone in a single well) containing 160 μl growth medium (TB containing 1% glucose and 50 μg/ml ampicillin) and incubated overnight at 37° C., shaking at 800 rpm. 150 μl of fresh TB medium containing 50 μg/ml ampicillin was inoculated with 8.5 μl of the overnight culture in a fresh 96-deep-well plate. After incubation for 120 minutes at 37° C. and 850 rpm, expression was induced with IPTG (0.5 mM final concentration) and continued for 4 hours. Cells were harvested and the pellets were frozen at -20° C. overnight before resuspension in 8.5 μl μl B-PERII (Thermo Scientific) and incubation for one hour at room temperature with shaking (600 rpm). Then, 160 μl PBS was added and cell debris was removed by centrifugation (3220 g for 15 min).

The extract of each lysed clone was applied as a 1:2000 dilution (final concentration) in PBSTB (PBS supplemented with 0.1% Tween 20® and 0.2% (w/v) BSA, pH 7.4) together with 2 nM (final concentration) biotinylated human 4-1 BB, 1:400 (final concentration) of anti-6His-D2 HTRF antibody—FRET acceptor conjugate (Cisbio) and 1:400 (final concentration) of anti-strep-Tb antibody FRET donor conjugate (Cisbio) to a well of 384 well plate and incubated for 75 minutes at RT. The HTRF was read-out on a Tecan M1000 using a 340 nm excitation wavelength and a 665±10 nm emission filter. Screening of several hundred clones by such a crude cell extract HTRF revealed hundreds of different ankyrin repeat domains with specificity for human 4-1 BB. Examples of amino acid sequences of selected ankyrin repeat domains that specifically bind to human 4-1 BB are provided in SEQ ID NO: 1 to 27.

These ankyrin repeat domains with binding specificity for human 4-1 BB and a negative control ankyrin repeat domain (SEQ ID NO: 42) with no binding specificity for human 4-1 BB were cloned into a pQE (QIAgen, Germany) based expression vector providing an N-terminal His-tag (SEQ ID NO: 43) to facilitate simple protein purification as described below. For example, expression vectors encoding the following ankyrin repeat proteins were constructed:

DARPin® protein #1 (SEQ ID NO:1 with a His-tag (SEQ ID NO:43) fused to its N terminus);

DARPin® protein #2 (SEQ ID NO:2 with a His-tag (SEQ ID NO:43) fused to its N terminus);

DARPin® protein #3 (SEQ ID NO:3 with a His-tag (SEQ ID NO:43) fused to its N terminus);

DARPin® protein #4 (SEQ ID NO:4 with a His-tag (SEQ ID NO:43) fused to its N terminus);

DARPin® protein #5 (SEQ ID NO:5 with a His-tag (SEQ ID NO:43) fused to its N terminus);

DARPin® protein #6 (SEQ ID NO:6 with a His-tag (SEQ ID NO:43) fused to its N terminus);

DARPin® protein #7 (SEQ ID NO:7 with a His-tag (SEQ ID NO:43) fused to its N terminus);

DARPin® protein #8 (SEQ ID NO:8 with a His-tag (SEQ ID NO:43) fused to its N terminus);

DARPin® protein #9 (SEQ ID NO:9 with a His-tag (SEQ ID NO:43) fused to its N terminus);

DARPin® protein #10 (SEQ ID NO:10 with a His-tag (SEQ ID NO:43) fused to its N terminus);

DARPin® protein #11 (SEQ ID NO:11 with a His-tag (SEQ ID NO:43) fused to its N terminus);

DARPin® protein #12 (SEQ ID NO:12 with a His-tag (SEQ ID NO:43) fused to its N terminus);

DARPin® protein #13 (SEQ ID NO:13 with a His-tag (SEQ ID NO:43) fused to its N terminus);

DARPin® protein #14 (SEQ ID NO:14 with a His-tag (SEQ ID NO:43) fused to its N terminus);

DARPin® protein #15 (SEQ ID NO:15 with a His-tag (SEQ ID NO:43) fused to its N terminus);

DARPin® protein #16 (SEQ ID NO:16 with a His-tag (SEQ ID NO:43) fused to its N terminus);

DARPin® protein #17 (SEQ ID NO:17 with a His-tag (SEQ ID NO:43) fused to its N terminus);

DARPin® protein #18 (SEQ ID NO:18 with a His-tag (SEQ ID NO:43) fused to its N terminus);

DARPin® protein #19 (SEQ ID NO:19 with a His-tag (SEQ ID NO:43) fused to its N terminus);

DARPin® protein #20 (SEQ ID NO:20 with a His-tag (SEQ ID NO:43) fused to its N terminus);

DARPin® protein #21 (SEQ ID NO:21 with a His-tag (SEQ ID NO:43) fused to its N terminus);

DARPin® protein #22 (SEQ ID NO:22 with a His-tag (SEQ ID NO:43) fused to its N terminus);

DARPin® protein #23 (SEQ ID NO:23 with a His-tag (SEQ ID NO:43) fused to its N terminus);

DARPin® protein #24 (SEQ ID NO:24 with a His-tag (SEQ ID NO:43) fused to its N terminus);

DARPin® protein #25 (SEQ ID NO:25 with a His-tag (SEQ ID NO:43) fused to its N terminus);

DARPin® protein #26 (SEQ ID NO:26 with a His-tag (SEQ ID NO:43) fused to its N terminus); and

DARPin® protein #27 (SEQ ID NO:27 with a His-tag (SEQ ID NO:43) fused to its N terminus).

Engineering of Additional Ankyrin Repeat Proteins with Binding Specificity for h4-1BB

Furthermore, pQE based expression vectors are constructed encoding the following ankyrin repeat proteins that specifically bind to human 4-1 BB:

DARPin® protein #28 (SEQ ID NO:28 with a His-tag (SEQ ID NO:43) fused to its N terminus);

DARPin® protein #29 (SEQ ID NO:29 with a His-tag (SEQ ID NO:43) fused to its N terminus);

DARPin® protein #30 (SEQ ID NO:30 with a His-tag (SEQ ID NO:43) fused to its N terminus);

DARPin® protein #31 (SEQ ID NO:31 with a His-tag (SEQ ID NO:43) fused to its N terminus);

DARPin® protein #32 (SEQ ID NO:32 with a His-tag (SEQ ID NO:43) fused to its N terminus).

DARPin® protein #33 (SEQ ID NO:33 with a His-tag (SEQ ID NO:43) fused to its N terminus);

DARPin® protein #34 (SEQ ID NO:34 with a His-tag (SEQ ID NO:43) fused to its N terminus);

DARPin® protein #35 (SEQ ID NO:35 with a His-tag (SEQ ID NO:43) fused to its N terminus).

DARPin® protein #36 (SEQ ID NO:36 with a His-tag (SEQ ID NO:43) fused to its N terminus);

DARPin® protein #37 (SEQ ID NO:37 with a His-tag (SEQ ID NO:43) fused to its N terminus); and

DARPin® protein #38 (SEQ ID NO:38 with a His-tag (SEQ ID NO:43) fused to its N terminus).

SEQ ID NO:28 to 38 are engineered based on the sequence of SEQ ID NO:1. In SEQ ID NO:28 to 38, the sequence of SEQ ID NO:1 is modified by replacing the RELLKA motif (positions 21 to 26) in the N-terminal capping module by RILLKA or RILLAA, by replacing a Valine (position 116) in the C-terminal capping module with Glutamic Acid, and/or by replacing the KAA motif (positions 124 to 126) in the C-terminal capping module by KLN. None of these modifications, alone or in combination, results in a significantly altered structure or functional properties of SEQ ID NO:28 to 38 as compared to SEQ ID NO:1.

High Level and Soluble Expression of 4-18B-Specific Ankyrin Repeat Proteins

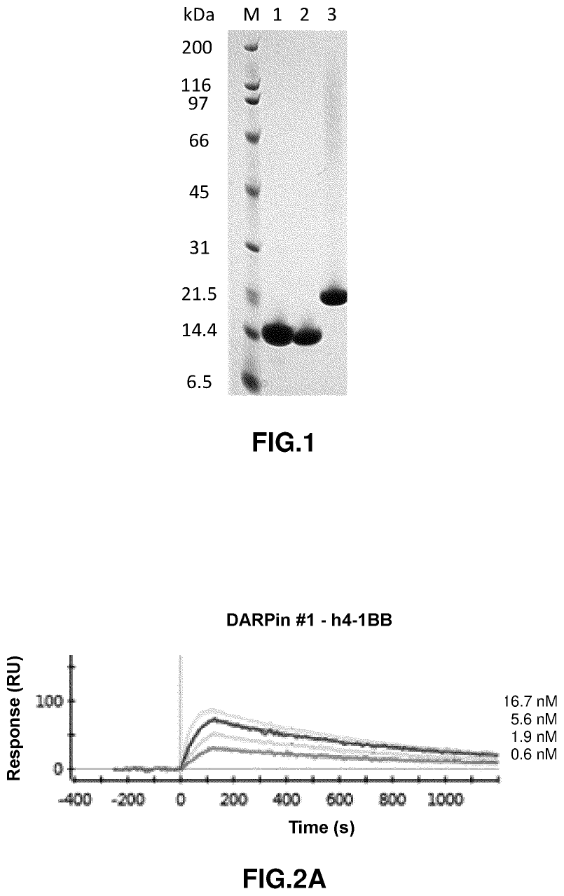

For further analysis, the selected clones showing specific 4-1 BB binding in the crude cell extract HTRF as described above were expressed in E. coli cells and purified using their His-tag according to standard protocols. 25 ml of stationary overnight cultures (TB, 1% glucose, 50 mg/l of ampicillin; 37° C.) were used to inoculate 500 ml cultures (TB, 50 mg/l ampicillin, 37° C.). At an absorbance of 1.0 to 1.5 at 600 nm, the cultures were induced with 0.5 mM IPTG and incubated at 37° C. for 4-5 h while shaking. The cultures were centrifuged and the resulting pellets were re-suspended in 25 ml of TBS₅₀₀ (50 mM Tris—HCl, 500 mM NaCl, pH 8) and lysed (sonication or French press). Following the lysis, the samples were mixed with 50 KU DNase/ml and incubated for 15 minutes prior to a heat-treatment step for 30 minutes at 62.5 ° C., centrifuged and the supernatant was collected and filtrated. Triton X100 (1% (v/v) final concentration) and imidazole (20 mM final concentration) were added to the homogenate. Proteins were purified over a Ni-nitrilotriacetic (Ni-NTA) acid column followed by a size exclusion chromatography on an ÄKTAxpress™ system according to standard protocols and resins known to the person skilled in the art. Alternatively, selected ankyrin repeat domains devoid of a His-tag are produced by high cell density fermentation in E. coli and purified by a series of chromatography and ultra/diafiltration steps according to standard resins and protocols known to the person skilled in the art. Highly soluble ankyrin repeat proteins with binding specificity for 4-1 BB were purified from E. coli culture (up to 200 mg ankyrin repeat protein per liter of culture) with a purity >95% as estimated from 4-12% SDS- PAGE. A representative example of such SDS-PAGE analysis is shown in FIG. 1.

### Example 2: Determination of Dissociation Constants (K_{D}) of Ankyrin Repeat Proteins with Binding Specificity for 4-1 BB by Surface Plasmon Resonance (SPR) Analysis

The binding affinities of the purified ankyrin repeat proteins on the human 4-1 BB target were analyzed using a ProteOn instrument (BioRad) and the measurement was performed according standard procedures known to the person skilled in the art.

Briefly, biotinylated human 4-1 BB was diluted in PBST (PBS, pH 7.4 containing 0.005% Tween 20®) and coated on a NLC chip (BioRad) to a level of around 320 resonance units (RU) (600 RU for single trace SPR measurements). The interaction of ankyrin repeat protein and h4-1 BB was then measured by injecting 200 μl running buffer (PBS, pH 7.4 containing 0.005% Tween 20®) containing serial dilutions of ankyrin repeat proteins covering a concentration range between 50 nM and 0.5 nM for multi-trace SPR measurements or 50 nM for single trace measurements (on-rate measurement), followed by a running buffer flow for at least 30 minutes at a constant flow rate of 100 μl/min (off-rate measurement). The regeneration was performed using 30 μl of 10 mM Glycine pH 2 followed by 30 μl of 124 mM H₃PO₄. The signals (i.e. resonance unit (RU) values) of the interspots and a reference injection (i.e. injection of running buffer only) were subtracted from the RU traces obtained after injection of ankyrin repeat protein (double-referencing). Based on the SPR traces obtained from the on-rate and off-rate measurements, the on- and off-rate of the corresponding ankyrin repeat protein—4-1 BB interaction was determined.

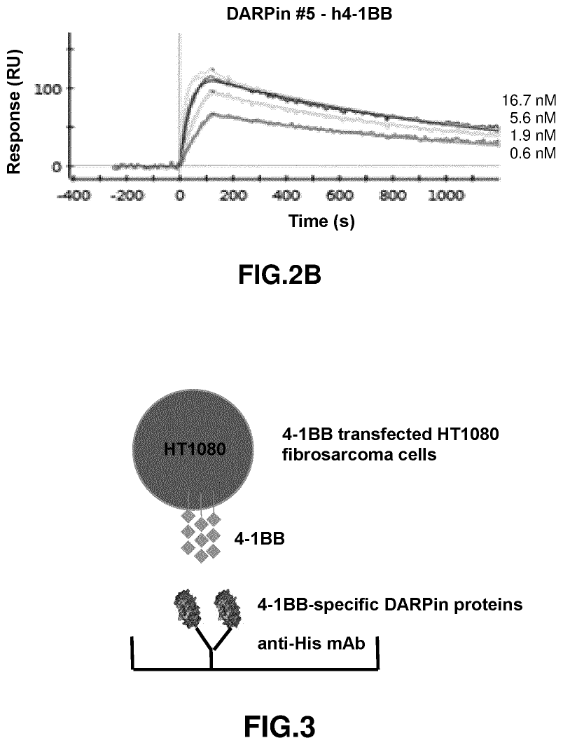

As representative examples, FIGS. 2A and 2B show SPR traces obtained for DARPin® protein #1 and DARPin® protein #5. Dissociation constants (K_{D}) were calculated from the estimated on- and off-rates using standard procedures known to the person skilled in the art. K_{D} values of the binding interactions of selected ankyrin repeat proteins with human 4-1 BB were determined to be in the range of 20 pM to 5 nM. Table 1 provides the K_{D}values of some selected ankyrin repeat proteins as examples. 4-1 BB-specific ankyrin repeat proteins of the invention also specifically bound to cynomolgus 4-1 BB.

TABLE 1
K_{D} values of ankyrin repeat
protein - human 4-1BB interactions

| DARPin ® | K_{D} |
|---|---|
| protein # | [nM] |
| #1 | 0.47 |
| #2 | 0.86 |
| #3 | 1.25 |
| #4 | 2.25 |
| #5 | 0.35 |
| #6 | 2.58 |
| #7 | 0.54 |
| #8 | 0.02 |
| #9 | 0.19 |
| #10 | 0.25 |
| #11 | 0.38 |
| #12 | 0.74 |
| #13 | 0.69 |
| #14 | 0.51 |
| #15 | 5.0 |
| #16 | 1.6 |
| #17 | 0.5 |
| #18 | 1.47 |
| #19 | 1.6 |
| #20 | 0.48 |
| #21 | 0.16 |
| #22 | 0.13 |
| #23 | 0.6 |
| #24 | 0.29 |
| #25 | 1.56 |
| #26 | 0.83 |
| #27 | 3.96 |

### Example 3: Activation of 4-1BB signaling in cells by 4-1BB-specific ankyrin repeat proteins

The ability of ankyrin repeat proteins with binding specificity for 4-1 BB to activate 4-1 BB signaling in cells was tested in a h4-1 BB-HT1080 reporter assay.

Generation of HT1080 Cells Expressing Human 4-1BB and an NF-κB-Regulated Luciferase Reporter Gene.

The fibrosarcoma cell line HT1080 (ATCC® CCL-121™) was transduced with a plasmid containing the cDNA of human 4-1 BB (Myc-DDK-tagged) obtained from OriGene Technologies (#RC200664), which contains the sequence of human 4-1 BB (Uniprot accession Q07011 or NCBI Ref. Seq. NM_001561) under control of a CMV-promoter and a neomycin resistance gene. Cells were cultured in Minimum Essential Medium (MEM) α medium+Glutamax supplemented with 10% (v/v) FBS and G418 (Geneticin®). 4-1 BB transduced HT1080 cells were assessed for human 4-1 BB expression by flow cytometry using the mouse anti-human 4-1 BB antibody clone 4B4-1 (BD Pharmingen™ Cat. No. 550890). The transfected cells were sorted by flow cytometry using the same antibody in order to enrich the population of h4-1 BB expressing HT1080 cells. The h4-1 BB HT1080 cells were further transfected with the NF-κB-luciferase reporter plasmid pNiFty3-N-Lucia (Invivogen, Cat. code pnf3-Ic2) containing a secreted luciferase reporter gene under control of NF-κB regulated mouse interferon beta minimal promoter and a Zeocin™ resistance gene using lipofectamine. Transfected cells were cultured in Minimum Essential Medium (MEM) α medium+Glutamax supplemented with 10% (v/v) FBS, G418 (Geneticin®), Zeocin™ (Invivogen, Cat. No. ant-zn-1) and Normocin™ (Invivogen, Cat. No. ant-nr-1). A population of h4-1 BB-HT1080-Lucia cells was used for the assays.

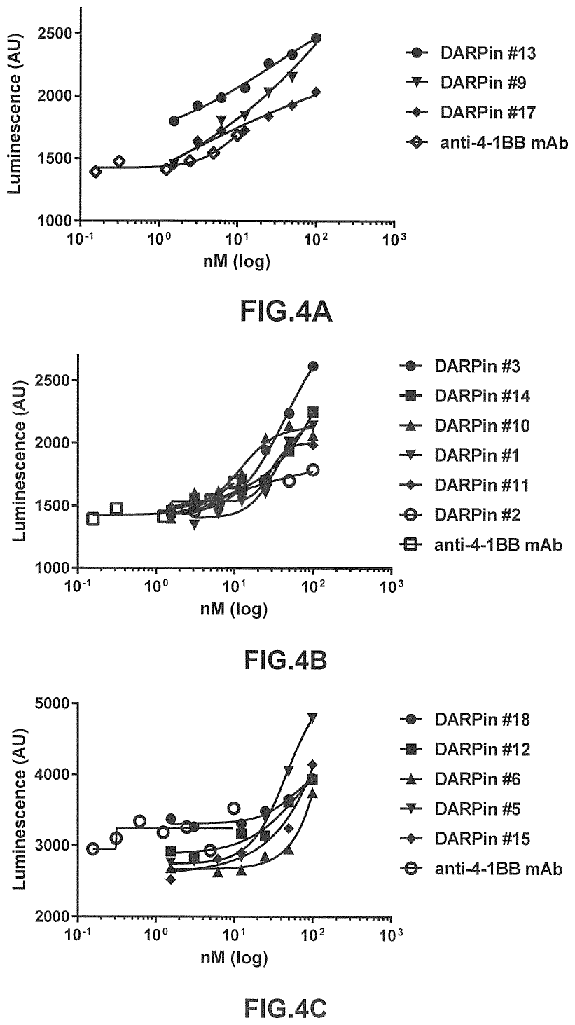

Assay set-up: NF-κB-luciferase human-4-1BB HT1080 cells were harvested and resuspended in MEMα medium+Glutamax supplied with 10% (v/v) FBS, 1% PenStrep, 1 mg/mL G418, 100 μg/mL Normocin and 100 μg/mL Zeocin. 96-well flat-bottom plates were coated with 30 nM anti-penta-His antibody (Qiagen, Cat. No. 34660) in PBS at 4° C. overnight. After washing three times with PBS, plates were blocked with PBS containing 1% FBS for 1 hour followed by incubation with increasing concentrations of ankyrin repeat proteins for 2 hours at room temperature in order to cross-link them on the plate. Then h4-1 BB-HT1080-luciferase cells were added and plates were incubated at 37° C., 5% CO₂ overnight. Supernatants were collected and centrifuged in a fresh 96-well plate. Triggering of 4-1 BB signaling in these cells leads to NFκB-mediated secretion of luciferase whose activity can be monitored using a luminescent substrate. QUANTI-Luc luciferase substrate (Invivogen, Cat. No. rep-qlc1) was mixed with the supernatant and luminescence read on a Tecan M1000 luminescence plate reader. The assay set-up is schematically illustrated in FIG. 3. Soluble (non-cross-linked) anti-4-1 BB monoclonal antibody (clone 20H4.9-IgG4) was included as a control.

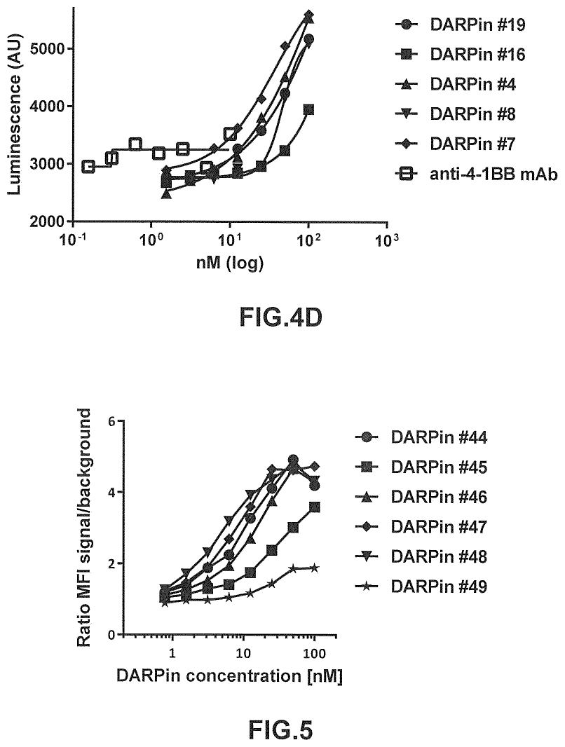

Results: The 4-1 BB-specific ankyrin repeat proteins activated 4-1 BB-mediated NF-κB signaling in the h4-1BB-expressing HT1080-luciferase cells in a concentration-dependent manner (FIGS. 4A to 4D). The results demonstrated that the disclosed ankyrin repeat proteins with binding specificity for 4-1 BB can activate 4-1 BB signaling in cells. The anti-4-1 BB monoclonal antibody (clone 20H4.9), which is thought to require Fc-cross-linking for full activity, was able to activate 4-1 BB signaling in soluble form to some degree.

### Example 4: 4-1BB-Specific Ankyrin Repeat Proteins Linked to a Localizer Molecule Bind to Cells and Activate 4-1BB Signaling Via Localizer-Mediated Clustering

Agonist-mediated clustering of 4-1BB on the cell surface is thought to be required or at least highly beneficial for effective activation of 4-1BB signaling. Such clustering and activation of 4-1BB can be mediated by 4-1 BB-specific ankyrin repeat proteins that are cross-linked by an antibody, as shown in Example 3. To test whether such clustering and activation of 4-1BB can also be mediated by a 4-1BB-specific ankyrin repeat protein that is linked to a localizer molecule, selected 4-1 BB-specific ankyrin repeat proteins were genetically linked to an example of a localizer molecule, a localizer that targets Tumor Antigen

A (hereinafter referred to as “TAA”), namely a TAA-specific ankyrin repeat domain. The resulting constructs comprised (1) an N-terminal His-tag (SEQ ID NO:43), (2) the TAA-specific localizer molecule, and (3) a 4-1 BB-specific ankyrin repeat protein connected via a peptide linker to the TAA-specific localizer molecule and located C-terminal of the TAA-specific localizer molecule within the construct (“TAA-4-1 BB DARPin® constructs”). The molecular identity of TAA is disclosed in U.S. provisional patent application no. 62/857,037 entitled “Multispecific Proteins” filed at the U.S. Patent & Trademark Office on 4 Jun. 2019 and assigned to Molecular Partners AG, and in the PCT international patent application claiming priority from U.S. 62/857,037 and filed on the filing date of the instant PCT application.

The TAA-4-1 BB DARPin® constructs were generated by cloning monovalent 4-1 BB-specific ankyrin repeat domains via BamHI and HindIII into the expression vector (pMPCME298, providing sequences encoding the TAA-specific ankyrin repeat domain and a peptide linker (SEQ ID NO:39), as well as an N-terminal His-tag (SEQ ID NO: 43) to facilitate protein purification), cut via Bsal and HindIII. Correct sequenced vectors containing the TAA-4-1 BB DARPin® constructs were transformed into inducible E. coli bacteria, expressed in 150 ml TB medium (containing 50 μg/ml ampicillin, induced with 0.5 mM IPTG at OD600˜1 and incubated for 6 h at 37° C.). Cells were lysed using sonication and proteins were purified using an IMAC benchtop purification with two triton wash steps.

The following TAA-4-1 BB DARPin® constructs were generated for further functional testing:

DARPin® protein #44 (comprising SEQ ID NO:1 as 4-1 BB DARPin® protein);

DARPin® protein #45 (comprising SEQ ID NO:2 as 4-1 BB DARPin® protein);

DARPin® protein #46 (comprising SEQ ID NO:3 as 4-1 BB DARPin® protein);

DARPin® protein #47 (comprising SEQ ID NO:4 as 4-1 BB DARPin® protein);

DARPin® protein #48 (comprising SEQ ID NO:5 as 4-1 BB DARPin® protein); and

DARPin® protein #49 (comprising SEQ ID NO:6 as 4-1 BB DARPin® protein).

As a negative control, an ankyrin repeat domain (SEQ ID NO: 42) with no binding specificity for human 4-1 BB was cloned into the vector, resulting in DARPin® protein #50:

DARPin® protein #50 (comprising SEQ ID NO:42 instead of a 4-1 BB specific ankyrin repeat domain).

TAA-4-188 DARPin® Constructs Bind to 4-1BB and TAA with High Affinity

The TAA-4-1 BB DARPin® constructs were investigated with SPR to obtain accurate affinity data for human 4-1 BB, cynomolgus 4-1 BB and human TAA targets.

Assay set-up: Briefly, SPR measurements were performed using a ProteOn XPR36 instrument (BioRad) as described above. Biotinylated human and cynomolgus 4-1 BB and human TAA were immobilized either directly or indirectly via NeutrAvidin (˜6000 RU pre-coated) on a GLC chip to reach 600 RU, 700 RU and 2000 RU, respectively. The interaction of TAA-4-1 BB DARPin® constructs to the coated targets was measured by injecting the DARPin® molecules in a serial dilution of 50, 16.7, 5.6, 1.9 and 0.6 nM with an association of 120 s and dissociation of 1800 s using a constant flow rate of 30 μl/min. Targets were regenerated between the individual measurements using 10 mM glycine pH 2 and 124 mM H₃P0₄. The signals were double referenced against the running buffer (PBST) treated control lane.

Screen: The results of the SPR measurements of TAA-4-1BB DARPin® constructs are summarized in Table 2. The TAA-4-1 BB DARPin® constructs showed binding affinities of 0.4 to 1.5 nM to human 4-1 BB, 1.1 to 2.9 nM to cynomolgus 4-1 BB and 0.1 to 0.4 nM to human TAA. The TAA-4-1 BB DARPin® constructs bound with higher affinity to hTAA than to h4-1BB. Cross-reactive binding to cynomolgus 4-1BB was confirmed for all tested TAA-4-1 BB DARPin® constructs, with at most about a 4-fold difference in binding affinity as compared to human 4-1 BB.

TABLE 2
K_{D} values of TAA-4-1BB DARPin ® interactions
with h4-1BB, c4-1BB and hTAA

| DARPin ® | K_{D} [nM] - | K_{D} [nM] - | K_{D} [nM] - |
|---|---|---|---|
| protein # | h4-1BB | C4-1BB | hTAA |
| #44 | 1.45 | 1.12 | 0.36 |
| #45 | 1.47 | n.d. | 0.33 |
| #46 | 1.15 | n.d. | 0.27 |
| #47 | 0.77 | 2.90 | 0.07 |
| #48 | 0.44 | n.d. | 0.17 |
| #49 | 0.86 | 1.48 | 0.17 |
| #50 | No binding | No binding | 0.22 |

TAA-4-188 DARPin® Constructs Bind to 4-18B-Expressing Cells

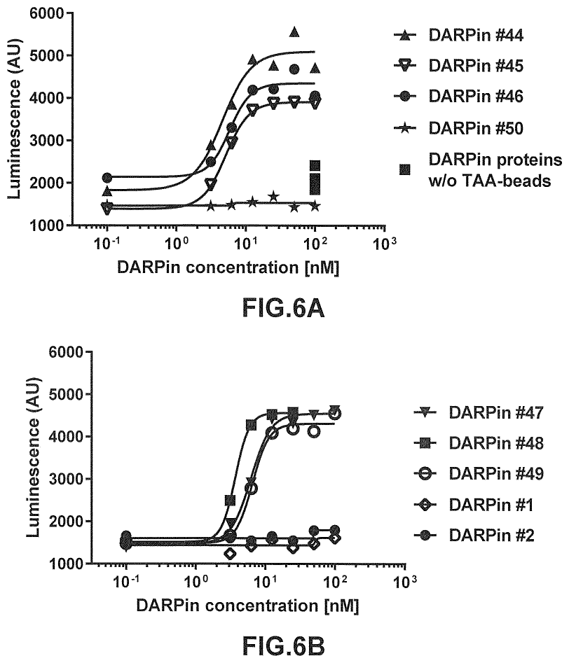

The TAA-4-1 BB DARPin® constructs were then tested for binding to ionomycin/PMA stimulated CEM cells. All the constructs bind to the CEM cells as depicted in FIG. 5.

Assay setup: Ankyrin repeat protein binding to cell membrane-bound human 4-1 BB on cells was measured by flow cytometry using the T lymphoblast cell line CCRF-CEM (ATCC® CCL-119™) stimulated with ionomcycin/PMA (phorbol 12-myristate 13-acetate) to express 4-1 BB. CEM cells cultured in RPMI medium supplemented with 10% (v/v) FBS+1% PenStrep were harvested and stimulated overnight in 96-well U-bottom plates using the same medium plus PMA (10 ng/ml) and ionomycin (500 ng/ml). Expression of 4-1 BB after stimulation was confirmed by flow cytometry using the mouse anti-human 4-1 BB antibody clone 4B4-1 (BD Pharmingen™, Cat. No. 550890). Stimulated CEM cells were harvested, then 30,000 cells were incubated with increasing concentrations of His-tagged ankyrin repeat proteins in FACS buffer (PBS+2% (v/v) FBS+0.1% sodium azide) for 30 minutes on ice. Cells were washed with FACS buffer and incubated with anti-penta-His Alexa Fluor-488 detection antibody (Qiagen, Cat. No. 35310) for 30 minutes on ice. After washing with PBS, cells were stained with Live/Dead Aqua dye (Thermo Fisher, No. L34957) and analyzed using a BD FACS Canto II or AttuneNxT cytometer.

Results: All TAA-4-1 BB DARPin® constructs, which show binding to recombinant 4-1 BB, also bind to cell membrane-expressed human 4-1 BB on activated CEM cells, as depicted in FIG. 5.

TAA-4-1BB DARPin® Constructs Activate 4-1BB Signaling in 4-1BB-Expressing Cells Mediated by Localizer-Induced Clustering

The TAA-4-1 BB DARPin® constructs were then further tested for their ability to activate 4-1 BB signaling in 4-1 BB-expressing cells mediated by clustering via the localizer. An assay was used that measures NF-κB reporter gene activation in HT1080 cells expressing human 4-1BB co-cultured in the presence of TAA-coated beads.

Assay setup: HT1080 cells expressing human-4-1 BB and an NF-κB-luciferase reporter gene (see Example 3) were harvested and resuspended in MEMα medium+Glutamax supplied with 10% (v/v) FBS, 1% PenStrep, 1mg/mL G418, 100 μg/mL Normocin and 100 μg/mL Zeocin. Using 96-well plates, 10,000 h4-1 BB-HT1080-luciferase cells were plated together with human TAA-coated beads and increasing concentrations of DARPin® proteins in the presence of TAA-biotin-coated streptavidin beads. Plates were incubated at 37° C., 5% CO₂ for 20 hours. Then supernatants were collected and centrifuged in a fresh 96-well plate. QUANTI-Luc reagent (Invivogen, Cat. No. rep-qlc1) was mixed with the supernatant and luminescence read on a Tecan M1000 luminescence plate reader. EC₅₀ values were determined by fitting the data with the four-parameter logistical fit model using Graphpad Prism software (version 7.02).

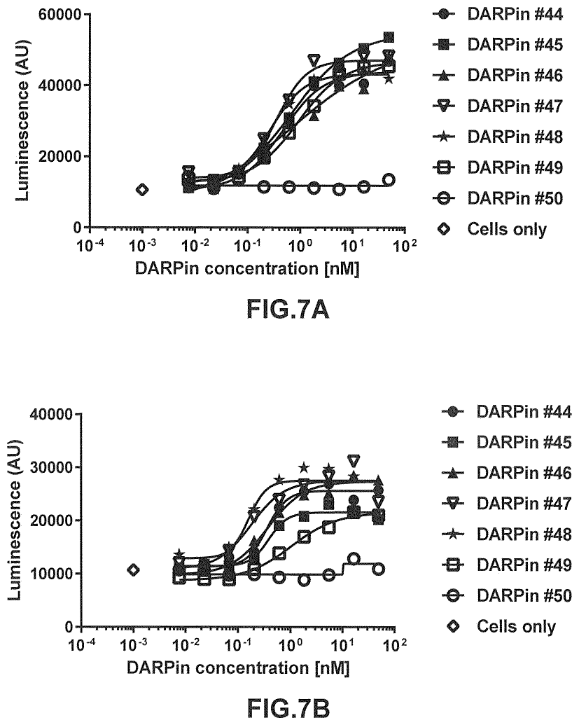

Results: This h4-1 BB-HT1080 luciferase reporter assay demonstrated that the disclosed 4-1 BB-specific ankyrin repeat proteins show 4-1 BB agonism in the presence of TAA-coated beads, if the 4-1 BB-specific ankyrin repeat domains are linked to a TAA-specific localizer (as in the TAA-4-1 BB DARPin® constructs) (See FIGS. 6A and 6B). The 4-1 BB agonism was dependent on localizer-mediated clustering, since in the absence of TAA-coated beads or in the absence of the localizer linked to the 4-1 BB-specific ankyrin repeat proteins, no agonism was observed. Table 3 provides the EC₅₀ values of selected TAA-4-1BB DARPin® constructs as examples:

TABLE 3
4-1BB activation in HT1080 cells in
presence of TAA-beads: EC₅₀ values of
TAA-4-1BB-specific DARPin ® proteins

| DARPin ® protein # | EC50 [nM] |
|---|---|
| #44 | 4.59 |
| #45 | 5.15 |
| #46 | 5.85 |
| #47 | 6.19 |
| #48 | 3.71 |
| #49 | 6.52 |

The TAA-4-1 BB DARPin® constructs were further tested for their ability to activate 4-1 BB signaling in 4-1 BB-expressing cells mediated by clustering via the localizer, using an assay that measures NFκKB reporter gene activation in 4-1 BB-expressing HT1080 cells co-cultured in the presence of TAA-expressing cells.

Generation of CHO Cells Expressing Human TAA

In brief, an expression vector (pMPMPA13) was generated by standard molecular biology techniques using a cDNA encoding human TAA. Chinese hamster ovary (CHO) cells (ATCC® CCL-121 ™) were transfected with the expression vector using Lipofectamine. Selection pressure was applied using different concentrations of Geneticin G-418 (Promega, V8091). Expression of hTAA was analysed by flow cytometry using an anti-TAA antibody. Two different populations (Population 1 and Population 2) of CHO-TAA transfectants were chosen for further experiments. FACS analysis demonstrated that CHO-TAA cells but not wildtype CHO cells (CHO-wt) express hTAA on the cell surface (data not shown).

Assay setup: h4-1BB-HT1080-luciferase cells as well as CHO-TAA cells were harvested and resuspended in MEMα medium+Glutamax supplied with 10% (v/v) FBS, 1% PenStrep, 1 mg/mL G418, 100 μg/mL Normocin™ and 100 μg/mL Zeocin™. Using 96-well plates, 40,000 h4-1BB-HT1080-luciferase cells and 40,000 CHO-TAA cells were plated and increasing concentrations of TAA-4-1 BB DARPin® constructs were added to the cells and incubated at 37° C., 5% CO₂. After 20 hours, supernatants were collected and centrifuged in a fresh 96-well plate. QUANTI-Luc reagent (Invivogen, Cat. No. rep-qlc1) was mixed with the supernatant and luminescence read on a Tecan M1000 luminescence plate reader. EC₅₀ values were determined by fitting the data with the four-parameter logistical fit model using Graphpad Prism software (version 7.02).

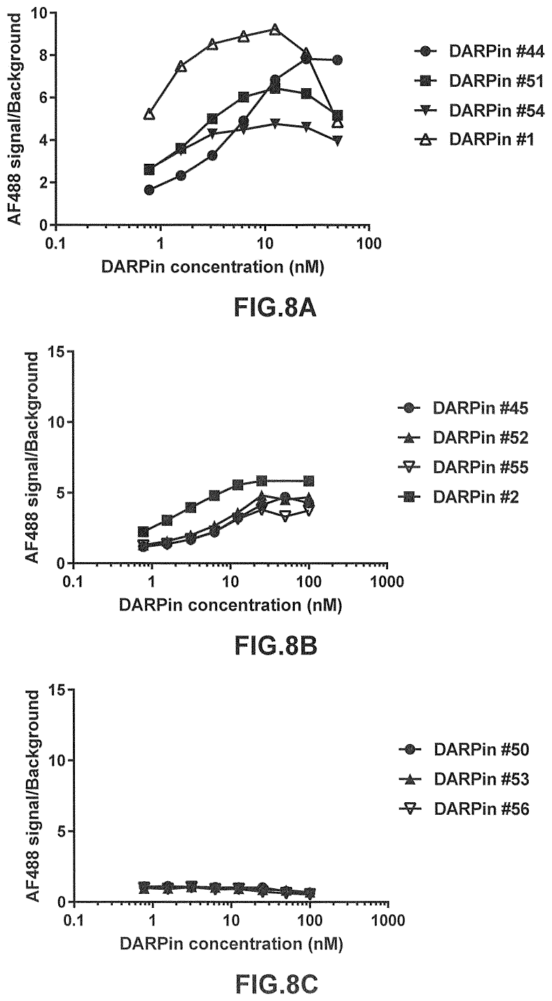

Results: FIG. 7A shows that in the presence of TAA-expressing cells (Population 1), all TAA-4-1BB DARPin® constructs induced 4-1BB signaling in the 4-1BB-expressing cells to a comparable extent, mediated by clustering via the localizer. DARPin protein #50, which does not bind to 4-1 BB, had no effect on 4-1BB signaling. Table 4 provides the EC₅₀ values of selected TAA-4-1BB DARPin® constructs as examples:

TABLE 4
4-1BB activation in HT1080 cells in presence of
TAA-expressing CHO cells (Population 1): EC₅₀
values of TAA-4-1BB-specific DARPin ® proteins

| DARPin ® protein # | EC50 [nM] |
|---|---|
| #44 | 0.47 |
| #45 | 0.67 |
| #46 | 0.73 |
| #47 | 0.36 |
| #48 | 0.28 |
| #49 | 0.91 |

Similar results were obtained with the second population of TAA-expressing cells (Population 2) (FIG. 7B). Table 5 provides the EC₅₀ values of selected TAA-4-1 BB DARPin® constructs as examples:

TABLE 5
4-1BB activation in HT1080 cells in presence of TAA-
expressing CHO cells (Population 2): EC₅₀ values of
TAA-4-1BB-specific DARPin ® proteins

| DARPin ® protein # | EC50 [nM] |
|---|---|
| #44 | 0.36 |
| #45 | 0.39 |
| #46 | 0.37 |
| #47 | 0.18 |
| #48 | 0.16 |
| #49 | 1.04 |

In conclusion, all tested TAA-4-1 BB DARPin® constructs were able to activate 4-1 BB signaling in 4-1 BB-expressing cells mediated by clustering via the localizer.

Example 5: The Presence of Two or Three 4-1BB-Specific Ankyrin Repeat Domains in a Protein Enhance its Potency in Activating 4-1BB Signaling

To test whether the presence of two or three 4-1 BB-specific ankyrin repeat domains in a protein (instead of just one) affects the potency of the protein in activating 4-1 BB signaling, multivalent ankyrin repeat protein constructs were assembled on DNA level using a Gibson assembly based approach and cloned into pMPAG06. Correctly assembled constructs were then transformed into inducible E. coli cells, expressed and purified using an IMAC benchtop purification with two triton wash steps, as described above.

The following bi- and tri-valent 4-1 BB ankyrin repeat protein constructs (and corresponding negative control constructs) were generated:

SEQ ID NO:51 (comprising two 4-1 BB ankyrin repeat domains (each derived from SEQ ID NO:1) connected by a PT-rich linker (SEQ ID NO:57));

SEQ ID NO:52 (comprising two 4-1 BB ankyrin repeat domains (each derived from SEQ ID NO:2) connected by a PT-rich linker (SEQ ID NO:57));

SEQ ID NO:53 (comprising two control ankyrin repeat domains with no binding specificity (each derived from SEQ ID NO: 42) connected by a PT-rich linker (SEQ ID NO:57));

SEQ ID NO:54 (comprising three 4-1BB ankyrin repeat domains (each derived from SEQ ID NO:1) connected by PT-rich linkers (SEQ ID NO:57));

SEQ ID NO:55 (comprising three 4-1BB ankyrin repeat domains (each derived from SEQ ID NO:2) connected by PT-rich linkers (SEQ ID NO:57)); and

SEQ ID NO:56 (comprising three control ankyrin repeat domains with no binding specificity (each derived from SEQ ID NO: 42) connected by PT-rich linkers (SEQ ID NO:57)).

These bi- and tri-valent 4-1 BB ankyrin repeat constructs (and corresponding negative control constructs) were genetically connected to a localizer molecule in order to test their potency in activating 4-1 BB signaling in cells. The following multivalent ankyrin repeat proteins were generated and tested:

DARPin® protein #51 (comprising the TAA-specific ankyrin repeat domain connected at its C-terminus to SEQ ID NO:51 by a peptide linker (SEQ ID NO:57));

DARPin® protein #52 (comprising the TAA-specific ankyrin repeat domain connected at its C-terminus to SEQ ID NO:52 by a peptide linker (SEQ ID NO:57));

DARPin® protein #53 (comprising the TAA-specific ankyrin repeat domain connected at its C-terminus to SEQ ID NO:53 by a peptide linker (SEQ ID NO:57));

DARPin® protein #54 (comprising the TAA-specific ankyrin repeat domain connected at its C-terminus to SEQ ID NO:54 by a peptide linker (SEQ ID NO:57));

DARPin® protein #55 (comprising the TAA-specific ankyrin repeat domain connected at its C-terminus to SEQ ID NO:55 by a peptide linker (SEQ ID NO:57)); and

DARPin® protein #56 (comprising the TAA-specific ankyrin repeat domain connected at its C-terminus to SEQ ID NO:56 by a peptide linker (SEQ ID NO:57)).

DARPin® protein #51 to DARPin® 56 also comprised an N-terminal His-tag (SEQ ID NO: 43) to facilitate simple protein purification.

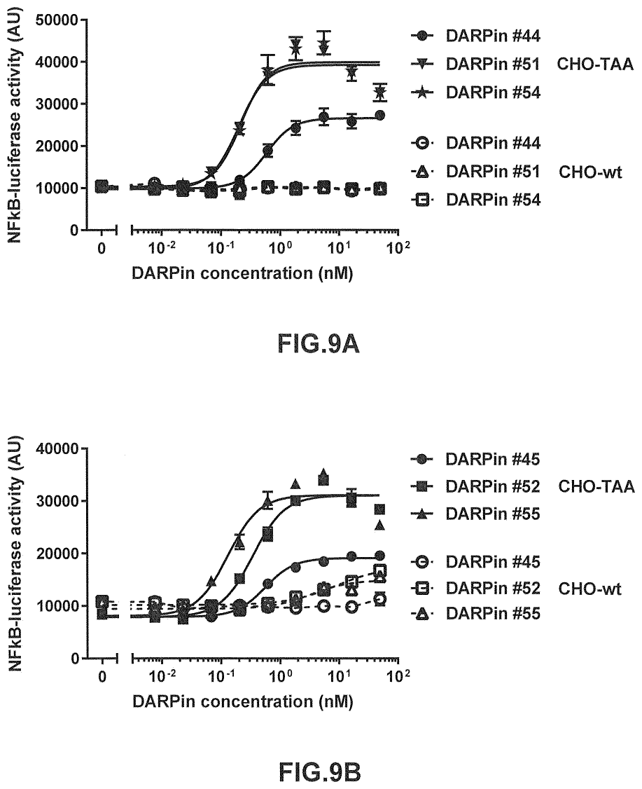

The multivalent ankyrin repeat proteins were tested, together with binding proteins comprising only one corresponding 4-1 BB-specific ankyrin repeat domain (with our without a linked localizer), for binding to PMA/ionomycin stimulated CEM cells. The assay was performed similarly as the one described in Example 4. As shown in FIGS. 8A to 8C, all of the ankyrin repeat proteins comprising one, two or three 4-1 BB-specific ankyrin repeat domains corresponding to SEQ ID NO:1 and all of the ankyrin repeat proteins comprising one, two or three 4-1 BB-specific ankyrin repeat domains corresponding to SEQ ID NO:2 bind to the CEM cells, while none of the negative control proteins lacking a 4-1 BB-specific ankyrin repeat domain binds to the cells.

The ability of these ankyrin repeat proteins to activate 4-1 BB signaling in cells was determined by a NF-κB-luciferase reporter assay in h4-1 BB-expressing HT1080 cells in the presence of TAA-expressing CHO cells. The assay was performed similarly as the one described in Example 4. The luminescence signal provided a relative measure of 4-1 BB signaling pathway activation.

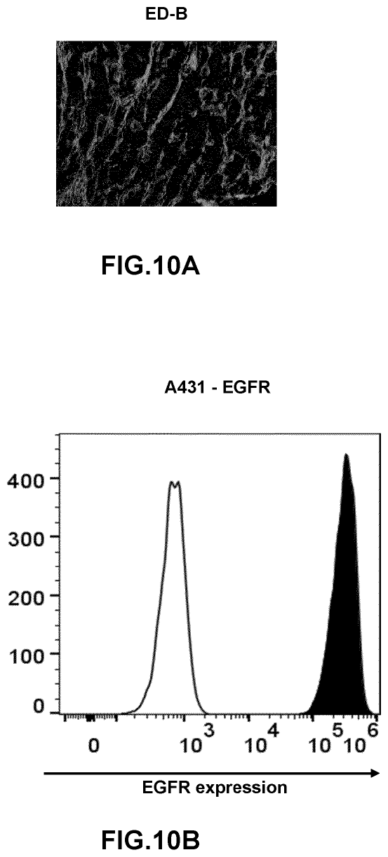

As shown in FIGS. 9A and 9B, binding proteins comprising two or three 4-1 BB-specific ankyrin repeat domains activated 4-1BB signaling stronger than binding proteins comprising only one corresponding 4-1 BB-specific ankyrin repeat domain. Surprisingly, binding proteins comprising three 4-1 BB ankyrin repeat domains did not activate 4-1 BB signaling more strongly than binding proteins comprising two 4-1 BB ankyrin repeat domains.

Thus, at least two 4-1 BB ankyrin repeat domains are required to obtain maximal 4-1 BB signaling activity, as measured by the NF-κB-Luciferase reporter assay. The addition of a third 4-1 BB ankyrin repeat domain does not confer higher potency to the binding proteins.

The binding proteins comprising one, two or three 4-1 BB ankyrin repeat domains corresponding to SEQ ID NO: 1 activated the 4-1 BB signaling pathway only in the presence of TAA-positive cells (CHO-TAA) and

TAA-mediated clustering. In the presence of TAA-negative cells (CHO-wt), and hence in the absence of TAA-mediated clustering, these binding proteins did not activate the 4-1BB pathway (FIG. 9A). The binding proteins comprising two or three 4-1 BB ankyrin repeat domains corresponding to SEQ ID NO: 2 showed some weak activation at higher concentrations in the presence of TAA-negative cells. However, the activation in the presence of TAA-positive cells was much higher (FIG. 9B). The negative control proteins did not show any activity (data no shown).

In conclusion, the potency of a 4-1BB ankyrin repeat protein to activate 4-1BB signaling in 4-1BB-expressing cells can be increased by including two or even three 4-1 BB ankyrin repeat domains in the protein.

### Example 6: 4-1BB-Specific Ankyrin Repeat Protein Linked to a Localizer Molecule Inhibits Tumor Growth and Selectively Increases Density of Human CD8 T Cells in the Tumor

The ability of a 4-1 BB-specific ankyrin repeat protein linked to a localizer molecule to stimulate T cells and inhibit tumor growth in vivo was tested in a HT-29 colon carcinoma xenograft model reconstituted with human peripheral blood mononuclear cells (PBMCs) (MiXeno). This humanized mouse model has been described as suitable for testing immune stimulatory efficacy of immune checkpoint and co-stimulatory drugs such as agonistic anti-4-1 BB antibodies. Treatment with the anti-4-1 BB mAb urelumab in this model is sufficient to significantly slow tumor growth. However, it also induces strong systemic effects such as accelerated graft versus host disease (GVHD) and liver T cell infiltration resulting in premature death compared to untreated mice. The model was used to assess whether a binding protein of the invention comprising a 4-1 BB-specific ankyrin repeat domain and a localizer molecule was able to increase intra-tumoral T cell infiltration and slow tumor growth while avoiding some of the non-tumor effects produced by anti-4-1 BB mAb urelumab, a non-targeted 4-1 BB agonistic monoclonal antibody. The binding protein used for the experiment (DARPin® protein #60) comprised SEQ ID NO: 51, wherein both L at the second last position and N at the last position of each of the two ankyrin repeat domains comprised in SEQ ID NO: 51 are substituted by A, connected at its N-terminus by a peptide linker (SEQ ID NO:57) to a TAA-specific ankyrin repeat domain as localizer.

Materials and Methods:

Tumor experiment: Immunodeficient NOG mice were inoculated subcutaneously in the right flank region with HT-29 tumor cells (3.5×10⁶). The mice were then humanized by injecting peripheral blood mononuclear cells (PBMCs) from two healthy human donors (3.5×10⁶ cells/ mouse). The test articles were administered to the tumor-bearing mice according to the predetermined regimen as shown in Table 6.

TABLE 6
Study design - experimental groups.

| | | Inoculum | | | | | |
|---|---|---|---|---|---|---|---|
| | | | PBMC 7 × 10⁶/ | | Dose | Dosing | Actual |
| Group | N | HT-29 (s.c.) | mouse (i.p.) | Treatment | (mg/kg) | Route | Schedule |
| 1 | 5 | 3.5 × 10⁶ cells | Donor 1 | Vehicle | 0 | i.v. | Q2D × 10 |
| | 5 | | Donor 2 | | | | |
| 2 | 5 | | Donor 1 | DARPin ® | 0.32 | i.v. | Q2D × 10 |
| | 5 | | Donor 2 | protein #60 | | | |

Date of tumor cell and PBMC inoculation was denoted as day 0. Tumor growth was monitored every 3 to 4 days. On day 18 of the experiment, mice were sacrificed, tumors removed, and studied by flow cytometry and quantitative immunofluorescence (QIF). Tumor growth analyses were limited to 18 days because mice started to show signs of graft-versus-host-disease (GVHD) after this time.

Flow cytometry: Data from raw FCS files were analyzed with FlowJo software (TreeStar). Cells were gated on live lymphocytes expressing the human surface markers CD45, CD4 and CD8. Dead cells were excluded from the analysis via incorporation of the live-dead labelling dye 7-AAD. Shown are the percentages of human CD8 T cells as percentage of total human CD45 positive cells detected in blood.

Immunohistochemistry: Tissues were recovered from mice at necropsy, and embedded in optimum cutting temperature compound (Sakura) and frozen without prior fixation. OCT embedded cryo-preserved specimens were cut into 7 μm sections and mounted on glass slides. The slides were fixed with cold acetone. The multiple immunofluorescence staining was performed with the following antibodies: anti-CD4 (Goat Pab, R&D System #AF-379-NA), anti-CD8 (Rabbit PAb, Abcam #ab40555) and anti-CD45 (clone H130, Biolegend #304002). These antibodies were respectively detected by anti-Sheep- Alexa Fluor® 647 (Thermofisher # A21448), anti-rabbit- Rhodamine Red™-X (Jackson ImmunoResearch #711-296-152) and anti-mouselgG1-Alexa Fluor® 488 (Jackson ImmunoResearch #115-545-205). The images were acquired on a Zeiss Axio Scan.Z1 slidescanner. The images were transferred with Zen blue software and analyzed using ImageJ 1.51n software, with FIJI package to quantify numbers of human CD45, CD8 and CD4 T cells.

Statistical Analysis: Statistical analyses were performed with the Prism 7.0.2 software (GraphPad Software). Tumor growth and body weight data were analyzed for statistically significant differences by using repeat measurement two-way ANOVA and Tukey's multiple comparison test (GraphPad Prism, Vers. 7.02). Survival curves were analyzed by the Kaplan-Meier method and compared by log-rank tests. Flow cytometry data at study end were analyzed using 1-way ANOVA (GraphPad Prism, Vers. 7.02). A two-tailed P<0.05 was considered as statistically significant.

Results

Tumor growth: Tumor growth was followed individually over time. In addition to the statistical analysis conducted on the data obtained at day 18 after tumor inoculation using Independent-Samples T test, tumor growth data were analyzed for statistically significant differences by using repeat measurement two-way ANOVA followed by Tukey's multiple comparison test. The tumor growth inhibition is summarized in Table 7.

TABLE 7
Summary of Anti-tumor Activity of Treatment

| | | Tumor Size | | | |
|---|---|---|---|---|---|
| | | at day 18 | TGI | | P |
| Group | Treatment | (mm³)^{a} | (%) | Significance^{b} | value^{b} |
| 1 | Vehicle | 477 ± 56 | — | | — |
| 2 | DARPin protein #60 | 349 ± 28 | 27 | ** | 0.0075 |

^{a}Mean ± SEM;
^{b}RM two-way ANOVA over all time points of tumor growth curves followed by Tukey’s
multiple comparison test vs. vehicle control (* p < 0.05, ** p < 0.001).

Analysis of the entire tumor growth curves gives higher power to the analysis compared to analysis of only the final tumor volume at the end of the study. The two analyses correlate well. Tumor growth was delayed in the DARPin® protein #60 treatment group (p<0.001). The vehicle administered had no significant impact on tumor growth. In summary, the test substance, DARPin® protein #60, demonstrated significant anti-tumor activity in the subcutaneous HT-29 human colon cancer MiXeno model.

Immunophenotyping of Blood and Tumors: To confirm the results obtained by flow cytometry, the human CD4 and CD8 T lymphocyte density was analyzed by histology in tumors excised on day 18. Histological examination was performed using tissues from 5 mice per group (data not shown). Treatment with DARPin® protein #60 led to denser infiltrates of human CD8 T lymphocytes in comparison with the vehicle group. The difference reached significance (P<0.01). On the other hand, numbers of CD4 tumor infiltrating lymphocytes were not significantly different across the groups.

Histological Analysis of Liver T Cell Infiltration: Histologic examination of livers excised on day 18 was performed using tissues from 5 mice per group. Quantification of infiltrates categorized as small, medium and large by surface area showed that treatment with DARPin® protein #60 did not induce an increase in liver T cell infiltration. This is in contrast to published results showing that administration of anti-4-1 BB mAb urelumab induced increased liver T cell infiltration by human PBMCs in NOG mice.

Conclusion

DARPin® protein #60 demonstrated anti-tumor activity in the subcutaneous HT-29 human colon cancer MiXeno model. Treatment with DARPin® protein #60 led to an increased density of human CD8 T cells in the tumor compared to vehicle-treated mice. Treatment with DARPin® protein #60 was well tolerated and did not lead to body weight loss or reduced survival and did not produce increased liver T cell infiltration compared to the vehicle group.

### Example 7: 4-1BB-Specific Ankyrin Repeat Proteins Linked to a Variety of Localizers Bind to and Activate 4-1BB in 4-1BB-Expressing Cells

To demonstrate that the ability of the 4-1 BB-specific ankyrin repeat proteins of the invention to activate 4-1BB signaling in cells is not dependent on the TAA-specific localizer used as an example in Examples 4 and 5, several other localizer-4-1 BB ankyrin repeat protein constructs were generated and tested.

Several different localizer molecules were genetically linked to a 4-1 BB-specific ankyrin repeat protein of the invention by using a Gibson assembly approach. Correct vectors were transformed into inducible E. coli expression cells, expressed and purified over a Ni-nitrilotriacetic (Ni-NTA) acid column followed by a size exclusion chromatography on an ÄKTAxpress™ system, as described above. These constructs comprised a localizer ankyrin repeat domain with binding specificity for fibronectin extra domain B (ED-B), epidermal growth factor receptor (EGFR) or human epidermal growth factor receptor 2 (HER2), respectively, connected by a PT-rich peptide linker to SEQ ID NO:51. The following localizer-4-1BB DARPin® constructs were generated, in addition to the TAA-4-1 BB DARPin® constructs described in the previous Examples:

DARPin® protein #57 (comprising an ED-B-specific ankyrin repeat protein connected at its C-terminus to SEQ ID NO:51 by a PT-rich peptide linker);

DARPin® protein #58 (comprising an EGFR-specific ankyrin repeat protein connected at its C-terminus to SEQ ID NO:51 by a PT-rich peptide linker); and

DARPin® protein #59 (comprising a HER2-specific ankyrin repeat protein connected at its C-terminus to SEQ ID NO:51 by a PT-rich peptide linker).

DARPin® proteins #57 to 59 also comprised an N-terminal His-tag (SEQ ID NO: 43) to facilitate simple protein purification.

Similar to the TAA-specific localizer used in Examples 4 and 5, these localizer ankyrin repeat domains each bind to a target (ED-B, EGFR or HER2) that is overexpressed in certain tumor types and therefore can be used for tumor localization. Specific binding of the localizer-4-1 BB DARPin® constructs to the respective localizer targets was demonstrated by SPR measurements (performed as described in previous Examples), each showing a binding affinity (K_{D}) below 10⁻⁸M to its respective recombinant target protein (data not shown). Table 8 provides the binding affinities (K_{D}) of various localizer-4-1 BB DARPin® constructs to human 4-1 BB:

TABLE 8
K_{D} values of various localizer-4-1BB DARPin ®
construct interactions with h4-1BB

| DARPin ® protein # | #51 | #57 | #58 | #59 |
|---|---|---|---|---|
| K_{D} [nM] - h4-1BB | <0.02* | <0.02* | <0.02* | <0.02* |

*accurate fit not possible due to slow off-rate

The ability of the localizer-4-1 BB DARPin® constructs to activate 4-1 BB signaling in 4-1 BB-expressing cells mediated by clustering via the localizer was tested in assays using 4-1 BB-expressing reporter cells co-cultured with cells expressing the localizer target EGFR or HER2, similar as described for the TAA-4-1 BB DARPin® constructs in Example 4, or in the presence of recombinant ED-B.

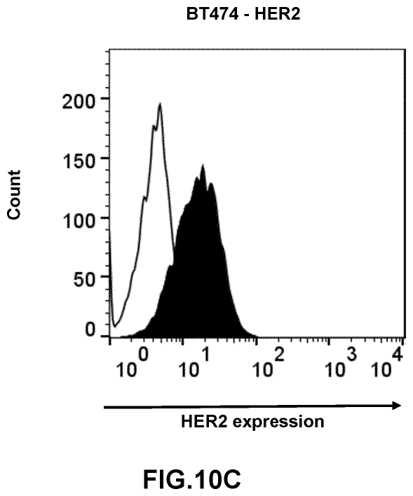

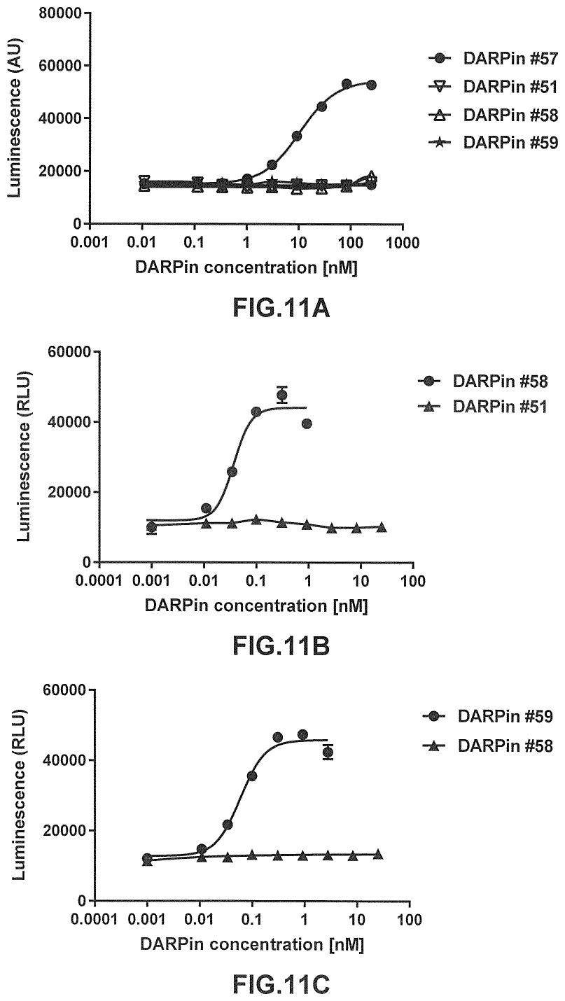

The expression of the localizer targets ED-B, EGFR and HER2 in tumor stroma (ED-B) or the respective tumor cells used for co-culture (A431 cells/EGFR and BT474 cells/HER2) is shown in FIGS. 10A to 10C. The localizer target-specific activation of 4-1 BB signaling in 4-1 BB-expressing reporter cells by the various localizer-4-1BB DARPin® constructs is shown in FIGS. 11A to 11C. In each of the experiments, a localizer-4-1 BB DARPin® construct was able to activate 4-1 BB signaling in the 4-1 BB-expressing cells, if the specific target for the localizer was present, as a cell surface protein on co-cultured tumor cells (FIG. 11B (EGFR) and FIG. 11C (HER2) or as a recombinant protein (FIG. 11A (ED-B)). For example, DARPin protein #58, which comprises an EGFR targeted localizer, was able to activate 4-1 BB signaling in 4-1 BB-expressing cells when co-cultured with EGFR-expressing A431 cells, but not when co-cultured with BT474 cells or in the presence of ED-B. These data demonstrate that the disclosed 4-1 BB-specific ankyrin repeat domains can be linked to different localizer molecules, resulting in functional localizer-4-1 BB DARPin® constructs that activate 4-1 BB signaling in 4-1 BB-expressing cells dependent on the presence of cells expressing the localizer target on their surface (EGFR and HER2) or dependent on the presence of the localizer target as an extracellular protein able to induce localizer-mediated clustering (ED-B).

The ability of a localizer-4-1 BB DARPin® construct to activate 4-1 BB signaling was also tested in 4-1 BB expressing primary CD8+ T-cells.

Materials and Methods:

In vitro human T cell IFNγ release assay using EGFR-clustering: Buffy coats were obtained from the Zurich blood donation centre and diluted with PBS. PBMCs were then isolated by density centrifugation using

Leucosep tubes. After several washing steps, CD8 T cells were purified from PBMCs using a negative selection human CD8 T cell isolation Kit according to the manufacturer's recommendations. CD8 T cells (1×10⁵/well) were seeded onto 96-well plates previously coated with 0.5 μg/ml anti-CD3 clone OKT-3 and Neutravidin followed by biotinylated EGFR in the presence of varying concentrations of localizer-4-1 BB DARPin® constructs. Cultures were incubated for 96 hours at 37° C., 5% CO₂ after which the supernatant was removed into a fresh 96-well plate and stored at −20° C. until analysis. The IFNγ concentration of the supernatant was detected using the human IFN-gamma DuoSet ELISA according to the manufacturer's instructions.

In vitro human T cell IFNγ release assay using HER2-clustering: This assay was performed as described above for EGFR-clustering, except that biotinylated HER2 was used instead of biotinylated EGFR.

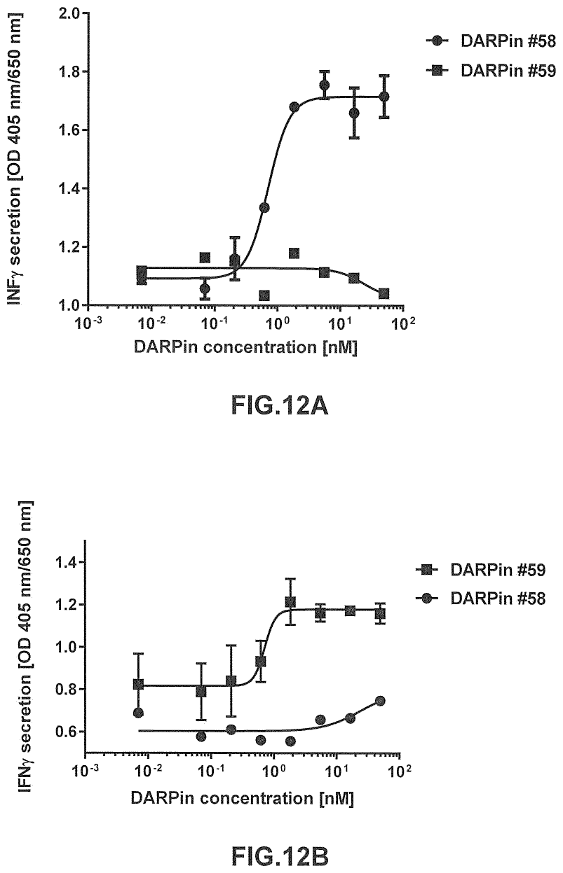

As shown in FIG. 12A, DARPin protein #58, which comprises an EGFR targeted localizer, induced interferon-γ secretion in the presence of EGFR-coated plate, while DARPin protein #59, which comprises a HER2 targeted localizer, had no effect in these circumstances. FIG. 12B shows that DARPin #59 induced interferon-γ secretion in the presence of HER2-coated plate, while DARPin protein #58 had no significant effect in these circumstances.

The results demonstrate that a 4-1 BB-specific binding protein of the invention comprising a localizer can activate 4-1 BB signaling in primary T-cells mediated by clustering via the localizer. In conclusion, 4-1 BB-specific ankyrin repeat proteins of the invention can be linked to a large variety of localizer molecules to generate proteins that bind to and activate 4-1BB signaling in 4-1BB-expressing cells mediated by clustering via the localizer.

1. A recombinant binding protein comprising an ankyrin repeat domain, wherein said ankyrin repeat domain has binding specificity for 4-1BB, and wherein said ankyrin repeat domain comprises an ankyrin repeat module comprising an amino acid sequence selected from the group consisting of (1) SEQ ID NOs: 58 to 71 and (2) one or more sequences in which up to 9 amino acids in any of SEQ ID NOs: 58 to 71 are substituted by another amino acid.

2. The binding protein of claim 1, wherein said ankyrin repeat module comprises an amino acid sequence selected from the group consisting of (1) SEQ ID NOs: 58, 59, 67, 68, 69 and (2) one or more sequences in which up to 9 amino acids in any of SEQ ID NOs: 58, 59, 67, 68, 69 are substituted by another amino acid.

3. The binding protein of claim 1, wherein said ankyrin repeat module is a first ankyrin repeat module and comprises an amino acid sequence selected from the group consisting of (1) SEQ ID NO: 58 and (2) one or more sequences in which up to 9 amino acids in SEQ ID NO: 58 are substituted by another amino acid, and wherein said ankyrin repeat domain further comprises a second ankyrin repeat module comprising an amino acid sequence selected from the group consisting of (1) SEQ ID NO: 59 and (2) one or more sequences in which up to 9 amino acids of SEQ ID NO: 59 are substituted by another amino acid.

4. The binding protein of claim 3, wherein said first ankyrin repeat module is located N-terminally of said second ankyrin repeat module within said ankyrin repeat domain.

5. A recombinant binding protein comprising an ankyrin repeat domain, wherein said ankyrin repeat domain has binding specificity for 4-1BB, and wherein said ankyrin repeat domain comprises an amino acid sequence with at least 80% amino acid sequence identity with any one of SEQ ID NOs: 1 to 38, wherein G at position 1 and/or S at position 2 of SEQ ID NOs: 1 to 38 are missing, and wherein L at the second last position and/or N at the last position of SEQ ID NOs: 2 to 4, 6-19, 25-27, 33-38 are substituted by A.

6. The binding protein of claim 5, wherein said ankyrin repeat domain comprises the amino acid sequence of SEQ ID NO: 1, wherein G at position 1 and/or S at position 2 of said ankyrin repeat domain are optionally missing.

7. The binding protein of claim 1, wherein said ankyrin repeat domain binds human 4-1BB in PBS with a dissociation constant (K_{D}) below 10⁻⁷M.

8. The binding protein of claim 1, wherein said recombinant binding protein comprises two or three ankyrin repeat domains with binding specificity for 4-1 BB.

9. The binding protein of claim 1, wherein said binding protein further comprises a localizer molecule.

10. A nucleic acid encoding the binding protein of claim 1.

11. A pharmaceutical composition comprising the binding protein of claim 1, and a pharmaceutically acceptable carrier and/or diluent.

12. A method of localized activation of 4-1 BB in 4-1 BB-expressing cells in a mammal, the method comprising the step of administering to said mammal the binding protein of claim 9.

13. A method of treating a medical condition, the method comprising the step of administering to a patient in need thereof a therapeutically effective amount of the binding protein of claim 9.

14. The method of claim 12, wherein said 4-1BB-expressing cells are located in a tumor.

15. The method of claim 13, wherein said medical condition is a cancer.

16. The binding protein of claim 5, wherein said ankyrin repeat domain binds human 4-1BB in PBS with a dissociation constant (K_{D}) below 10⁻⁷M.

17. The binding protein of claim 5, wherein said recombinant binding protein comprises two or three ankyrin repeat domains with binding specificity for 4-1BB.

18. The binding protein of claim 5, wherein said binding protein further comprises a localizer molecule.

19. A nucleic acid encoding the binding protein of claim 5.

20. A pharmaceutical composition comprising the binding protein of claim 5 and a pharmaceutically acceptable carrier and/or diluent.

21. A pharmaceutical composition comprising the nucleic acid of claim 10 and a pharmaceutically acceptable carrier and/or diluent.

22. A method of claim 12, wherein the mammal is a human.

23. The method of claim 13, wherein said 4-1BB-expressing cells are located in a tumor.


SEQUENCE LISTING (81 sequences)

SEQ ID NO: 1 - protein, 126 aa, Artificial Sequence
  ankyrin repeat domain
GSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKAA

SEQ ID NO: 2 - protein, 126 aa, Artificial Sequence
  ankyrin repeat domain
GSDLGKKLLVAANVGQDDEVRILLAAGADVNAKDYHGYTPLHHAATYGHLEIVEVLLKAGADVNAKDQTGLTPLHLAAAKGHLEIVEVLLKAGADVNAQDHHGYTPADLAAFVGHEDIAVVLQKLN

SEQ ID NO: 3 - protein, 126 aa, Artificial Sequence
  ankyrin repeat domain
GSDLGRKLLQAAQLGQDDEVRELLKAGADVNAKDSRGITPLHVAAWQGHLEIVEVLLKASADVNAKDHAGITPLHLAAFLGHLEIVEVLLKAGADVNAQDQEGRTPADLAALQGHEDIAKVLQKLN

SEQ ID NO: 4 - protein, 159 aa, Artificial Sequence
  ankyrin repeat domain
GSDLGKKLLEAARAGQDDEVRELLKAGADVNAKDTYGETPLHLAAWKGHLEIVEVLLKAGADVNAKDSIGITPLHVAAHHGHLEIVEVLLKAGADVNAKDKWGRTPLHLAALLGHLEIAEVLLKAGADVNAQDKSGKTPADLAADAGHDDIAEVLQKLN

SEQ ID NO: 5 - protein, 160 aa, Artificial Sequence
  ankyrin repeat domain
GSDLGKKLLEAARAGQDDEVRELLKAGADVNAKDQEHGSTPLHLAANYGHLEIVEVLLKAGADVNAKNWWGNTPLHLAAVYGHLEIVEVLLKAGADVNAKDNHGHTPLHLAAWYGHLEIVEVLLKAGADVNAQDKSGKTPADLAADAGHEDIAEVLQKAA

SEQ ID NO: 6 - protein, 126 aa, Artificial Sequence; identical to SEQ ID NO: 2
  ankyrin repeat domain

SEQ ID NO: 7 - protein, 126 aa, Artificial Sequence
  ankyrin repeat domain
GSDLGSKLLNAARKGQDDEVRILLAAGADVNAKDYYGQTPLHLAAVFGHLEIVEVLLKAGADVNAKDVLGRTPLHVAAYAGHLEIAEVLLKAGADVNAQDFDGFTPADLAAYVGLEDIAEVLQKLN

SEQ ID NO: 8 - protein, 159 aa, Artificial Sequence
  ankyrin repeat domain
GSDLGKKLLEAARAGQDDEVRELLKAGADVNAKDRFGSTPLHIAAREGHLEIVEVLLKAGADVNAKDSWGATPLHIAAWRGHLEIVEVLLKAGADVNAKDRWGRTPLHIAAWTGHLEIVEVLLKACADVNAQDKSGKTPADLAADVGLEDIAEVLQKLN

SEQ ID NO: 9 - protein, 93 aa, Artificial Sequence
  ankyrin repeat domain
GSDLGEKLLHAAYYGQDDEVRILLAAGADVNAKDNIGVTPLHFAAYFGHLEIVEVLLKAGADVNAQDYYGKTPADLAAYHGHEDIAEVLQKLN

SEQ ID NO: 10 - protein, 93 aa, Artificial Sequence
  ankyrin repeat domain
GSDLGDKLLHAAYYGQDDEVRILLAAGADVNAKDNIGVTPLHFAAYFGHLEIVEVLLKAGADVNAQDYYGKTPADLAAYHGHEDIAEVLQKLN

SEQ ID NO: 11 - protein, 126 aa, Artificial Sequence
  ankyrin repeat domain
GSDLGWKLLSAAWQGQDDEVRILLAAGADVNAKDTFGTTPLHWAAHAGHLEIVEVLLKAGADVNAKDQWGTTPLHYAAYYGHLEIVEVLLKAGADVNAQDDKGFTPADLAAIVGHEDIAEVLQKLN

SEQ ID NO: 12 - protein, 126 aa, Artificial Sequence
  ankyrin repeat domain
GSDLGQKLLHTAYDGQDDEVRILLAAGADVNAKDFFGTTPLHWAAHAGHLEIVEVLLKAGADVNAKDQWGTTPLHYAAYYGHLEIVEVLLKAGADVNAQDDKGFTPADLAAIVGHEDIAEVLQKLN

SEQ ID NO: 13 - protein, 126 aa, Artificial Sequence
  ankyrin repeat domain
GSDLGLKLLYAAAEGQDDEVRILLAAGADVNAKDTRGQTPLHYAAWFGHLEIVEVLLKAGADVNAKDWSGDTPLHVAAWRGHLEIVEVLLKAGADVNAQDNSGNTPADLAAWIGHEDIAEVLQKLN

SEQ ID NO: 14 - protein, 126 aa, Artificial Sequence
  ankyrin repeat domain
GSDLGQKLLYAAFYGQDDEVRILLAAGADVNAKDFFGTTPLHWAAHAGHLEIVEVLLKAGADVNAKDQWGTTPLHYAAYYGHLEIVEVLLKAGADVNAQDDKGFTPADLAAIVGHEDIAEVLQKLN

SEQ ID NO: 15 - protein, 126 aa, Artificial Sequence
  ankyrin repeat domain
GSDLGQKLLYAAYYGQDDEVRILLAAGADVNAKDFFGTTPLHWAAHAGHLEIVEVLLKAGADVNAKDQWGTTPLHYAAYQGHLEIVEVLLKAGADVNAQDTTGATPADLAAQAGHEDIAEVLQKLN

SEQ ID NO: 16 - protein, 159 aa, Artificial Sequence
  ankyrin repeat domain
GSDLGAKLLNAAFFGQDDEVRILLAAGADVNAKDDYGHTPLHWAAVTGHLEIVEVLLKAGADVNAKDWLGETPLHHAAYEGHLEIVEVLLKAGADVNAQDEQGETPADLAAYEGHEDIAEVLLKAGADVNAQDDKGFTPADLAAIVGHEDIAEVLQKLN

SEQ ID NO: 17 - protein, 126 aa, Artificial Sequence
  ankyrin repeat domain
GSDLGEKLLYAAYYGQDDEVRILLAAGADVNAKDHVGLTPLHYAAAIGHLEIVEVLLKAGADVNAKDHYGYTPLHYAAYKGHLEIVEVLLKAGADVNAQDSAGHTPADLAAHHGHEDIAEVLQKLN

SEQ ID NO: 18 - protein, 126 aa, Artificial Sequence
  ankyrin repeat domain
GSDLGYKLLYAAAYGQDDEVRILLAAGADVNAKDQYGETPLHHAASHGHLEIVEVLLKAGADVNAKDTLGDTPLHLAAVVGHLEIVEVLLKAGADVNAQDTEGKTPADLAAYTGHEDIAEVLQKLN

SEQ ID NO: 19 - protein, 159 aa, Artificial Sequence
  ankyrin repeat domain
GSDLGKKLLEAARAGQDDEVRELLKAGADVNAKDTHGWTPLHVAALEGHLEIVEVLLKAGAYVNAKDEWGQTPLHVAAWRGHLEIVEVLLKAGADVNAKDRYGWTPLHIAAWLGHLEIAEVLLKAGADVNAQDKSGKTPADLAADAGQEDIVEVLQKLN

SEQ ID NO: 20 - protein, 126 aa, Artificial Sequence
  ankyrin repeat domain
GSDLGEKLLHAAYYGQDDEVRILLAAGADVNAKDDQGLTPLHLAAAIGHLEIVEVLLKAGADVNAKDAYGETPLHLAARRGHLEIVEVLLKAGADVNAQDKSGKTPADLAADAGHEDIAEVLQKAA

SEQ ID NO: 21 - protein, 126 aa, Artificial Sequence
  ankyrin repeat domain
GSDLGEKLLTAAYYGQDDEVRILLAAGADVNAKDKQGLTPLHYAAAVGHLEIVEVLLKAGTDVNAKDFYGETPLHLAASRGHLEIVEVLLKAGADVNAQDKSGKTPADLAADAGHEDIAEVLQKAA

SEQ ID NO: 22 - protein, 126 aa, Artificial Sequence
  ankyrin repeat domain
GSDLGEKLLWAAWEGQGDEVRILLAAGADVNAKDLTGRTPLHFAAHSGHLEIVEVLLKAGADVNAKDNAGRTPLHLAAELGHLEIVEVLLKAGADVNAQDKSGKTPADLAADAGHEDIAEVLQKAA

SEQ ID NO: 23 - protein, 126 aa, Artificial Sequence
  ankyrin repeat domain
GSDLGEKLLWAAYLGQDDEVRILLAAGADVNAKDATGRTPLHFAAHSGHLEIVEVLLKAGADVNAKDNAGRTPLHLAAELGHLEIVEVLLKAGADVNAQDKSGKTPADLAADAGHEDIAEVLQKAA

SEQ ID NO: 24 - protein, 126 aa, Artificial Sequence
  ankyrin repeat domain
GSDLGEKLLNAAYYGQDDEVRILLAAGADVNAKDVKGETPLHYAAAVGHLEIVEVLLKAGADVNAKDQYGATPLHLAAYKGHLEIVEVLLKAGADVNAQDKSGKTPADLAADAGHEDIAEVLQKAA

SEQ ID NO: 25 - protein, 159 aa, Artificial Sequence
  ankyrin repeat domain
GSDLGKKLLEAARAGQDDEVRELLKAGADVNAKDEWGETPLHLAAWKGHLEIVEVLLKAGADVNAKDSIGITPLHVAAHHGHLEIVEVLLKAGADVNAKDKWGRTPLHLAALLGHLEIVEVLLKAGADVNAQDKSGKTPADLAADAGREDIAEVLQKLN

SEQ ID NO: 26 - protein, 159 aa, Artificial Sequence
  ankyrin repeat domain
GSDLGKKLLEAARAGQDDEVRELLKAGADVNAKDHAGQTPLHIAALRGHLEIVEVLLKAGADVNAKDKYGQTPLHLAAILGHLEIVEVLLKAGADVNAKDQEGDTPLHIAAWQGHLEIVEVLLKAGADVNAQDKSGKTPADLAADAGHEDITEVLQKLN

SEQ ID NO: 27 - protein, 159 aa, Artificial Sequence
  ankyrin repeat domain
GSDLGKKLLEAARAGQDDEVRELLKAGADVNAKDHQGATPLHNAAWAGHLEIVEVLLKAGADVNAKDKAGYTPLHIAAITGHLEIVEVLLKAGADVNAKDQWGKTPLHLAAQVGHLEIVEVLLKAGADVNAQDKSGKTPADLAADAGHEDIAKVLQKLN

SEQ ID NO: 28 - protein, 126 aa, Artificial Sequence
  Ankyrin repeat domain
GSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHEDIAEVLQKAA

SEQ ID NO: 29 - protein, 126 aa, Artificial Sequence
  Ankyrin repeat domain
GSDLGQKLLKAAQEGQDDEVRILLKAGADVNAKDLRGITPLHVAAWQGHLEIVEVLLKAGADVNAKDSKGETPLHLAAFRGHLEIVEVLLKAGADVNAQDQQGETPADLAALAGHEDIAEVLQKAA

SEQ ID NO: 30 - protein, 126 aa, Artificial Sequence
  Ankyrin repeat domain
GSDLGQKLLKAAQEGQDDEVRILLAAGADVNAKDLRGITPLHVAAWQGHLEIVEVLLKAGADVNAKDSKGETPLHLAAFRGHLEIVEVLLKAGADVNAQDQQGETPADLAALAGHEDIAEVLQKAA

SEQ ID NO: 31 - protein, 126 aa, Artificial Sequence
  Ankyrin repeat domain
GSDLGQKLLKAAQEGQDDEVRILLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKAA

SEQ ID NO: 32 - protein, 126 aa, Artificial Sequence
  Ankyrin repeat domain
GSDLGQKLLKAAQEGQDDEVRILLAAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKAA

SEQ ID NO: 33 - protein, 126 aa, Artificial Sequence
  Ankyrin repeat domain
GSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKLN

SEQ ID NO: 34 - protein, 126 aa, Artificial Sequence
  Ankyrin repeat domain
GSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHEDIAEVLQKLN

SEQ ID NO: 35 - protein, 126 aa, Artificial Sequence
  Ankyrin repeat domain
GSDLGQKLLKAAQEGQDDEVRILLKAGADVNAKDLRGITPLHVAAWQGHLEIVEVLLKAGADVNAKDSKGETPLHLAAFRGHLEIVEVLLKAGADVNAQDQQGETPADLAALAGHEDIAEVLQKLN

SEQ ID NO: 36 - protein, 126 aa, Artificial Sequence
  Ankyrin repeat domain
GSDLGQKLLKAAQEGQDDEVRILLAAGADVNAKDLRGITPLHVAAWQGHLEIVEVLLKAGADVNAKDSKGETPLHLAAFRGHLEIVEVLLKAGADVNAQDQQGETPADLAALAGHEDIAEVLQKLN

SEQ ID NO: 37 - protein, 126 aa, Artificial Sequence
  Ankyrin repeat domain
GSDLGQKLLKAAQEGQDDEVRILLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKLN

SEQ ID NO: 38 - protein, 126 aa, Artificial Sequence
  Ankyrin repeat domain
GSDLGQKLLKAAQEGQDDEVRILLAAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKLN

SEQ ID NO: 39 - protein, 24 aa, Artificial Sequence
  PT-linker
GSPTPTPTTPTPTPTTPTPTPTGS

SEQ ID NO: 40 - protein, 5 aa, Artificial Sequence
  GS-linker
  MISC_FEATURE: [Gly-Gly-Gly-Gly-Ser]n, wherein n is 1, 2, 3, 4, 5, or 6
GGGGS

SEQ ID NO: 41 - DNA, 378 bp, Artificial Sequence
  ankyrin repeat domain
ggcagcgacttgggtcagaagttgttgaaagcggcacaggagggtcaggacgatgaggtccgtgaattgctgaaggccggcgcagacgtgaatgcaaaggatctgcgtggcattaccccgctgcacgtggcggcctggcagggtcacctggaaattgttgaggttttactgaaagctggcgcggacgttaacgctaaagacagcaaaggtgaaaccccgttacatctggcggcattccgtggccacttggagatcgttgaggtgctgctgaaggctggtgccgacgtcaatgctcaagatcaacaaggtgaaacgccagcagacttggcagcgctggctggtcatgtcgatatcgcggaggtactgcagaaagcggcg

SEQ ID NO: 42 - protein, 126 aa, Artificial Sequence
  Ankyrin repeat domain
GSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAA

SEQ ID NO: 43 - protein, 10 aa, Artificial Sequence
  His-tag
MRGSHHHHHH

SEQ ID NO: 44 - protein, 32 aa, Artificial Sequence
  N-cap
GSDLGQKLLKAAQEGQDDEVRELLKAGADVNA

SEQ ID NO: 45 - protein, 32 aa, Artificial Sequence
  N-cap
GSDLGQKLLKAAQEGQDDEVRILLKAGADVNA

SEQ ID NO: 46 - protein, 32 aa, Artificial Sequence
  N-cap
GSDLGQKLLKAAQEGQDDEVRILLAAGADVNA

SEQ ID NO: 47 - protein, 32 aa, Artificial Sequence
  N-Cap
  misc_feature (6)..(6): Xaa can be any naturally occurring amino acid
  misc_feature (10)..(10): Xaa can be any naturally occurring amino acid
  misc_feature (13)..(14): Xaa can be any naturally occurring amino acid
GSDLGXKLLXAAXXGQDDEVRILLAAGADVNA

SEQ ID NO: 48 - protein, 33 aa, Artificial Sequence
  Randomized repeat module
  misc_feature (1)..(1): Xaa can be any naturally occurring amino acid
  misc_feature (3)..(4): Xaa can be any naturally occurring amino acid
  misc_feature (6)..(6): Xaa can be any naturally occurring amino acid
  misc_feature (14)..(15): Xaa can be any naturally occurring amino acid
  misc_feature (27)..(27): Xaa can be any naturally occurring amino acid
XDXXGXTPLHLAAXXGHLEIVEVLLKXGADVNA

SEQ ID NO: 49 - protein, 33 aa, Artificial Sequence
  Randomized repeat module
  misc_feature (3)..(4): Xaa can be any naturally occurring amino acid
  misc_feature (6)..(6): Xaa can be any naturally occurring amino acid
  misc_feature (14)..(15): Xaa can be any naturally occurring amino acid
KDXXGXTPLHLAAXXGHLEIVEVLLKAGADVNA

SEQ ID NO: 50 - protein, 33 aa, Artificial Sequence
  Randomized repeat module
  misc_feature (3)..(4): Xaa can be any naturally occurring amino acid
  misc_feature (6)..(6): Xaa can be any naturally occurring amino acid
  misc_feature (11)..(11): Xaa can be any naturally occurring amino acid
  misc_feature (14)..(15): Xaa can be any naturally occurring amino acid
KDXXGXTPLHXAAXXGHLEIVEVLLKAGADVNA

SEQ ID NO: 51 - protein, 274 aa, Artificial Sequence
  Protein comprising two ankyrin repeat domains
GSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKLNGSPTPTPTTPTPTPTTPTPTPTGSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKLN

SEQ ID NO: 52 - protein, 274 aa, Artificial Sequence
  Protein comprising two ankyrin repeat domains
GSDLGDKLLHAAYYGQDDEVRILLAAGADVNAKDTQGLTPLHLAAAIGHLEIVEVLLKAGADVNAKDAYGETPLHLVASRGHLEIVEVLLKAGADVNAQDYSGRTPADLAAEYGHEDIAEVLQKLNGSPTPTPTTPTPTPTTPTPTPTGSDLGDKLLHAAYYGQDDEVRILLAAGADVNAKDTQGLTPLHLAAAIGHLEIVEVLLKAGADVNAKDAYGETPLHLVASRGHLEIVEVLLKAGADVNAQDYSGRTPADLAAEYGHEDIAEVLQKLN

SEQ ID NO: 53 - protein, 274 aa, Artificial Sequence
  Protein comprising two ankyrin repeat domains
GSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKLNGSPTPTPTTPTPTPTTPTPTPTGSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKLN

SEQ ID NO: 54 - protein, 422 aa, Artificial Sequence
  Protein comprising three ankyrin repeat domains
GSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKLNGSPTPTPTTPTPTPTTPTPTPTGSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKLNGSPTPTPTTPTPTPTTPTPTPTGSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKLN

SEQ ID NO: 55 - protein, 422 aa, Artificial Sequence
  Protein comprising three ankyrin repeat domains
GSDLGDKLLHAAYYGQDDEVRILLAAGADVNAKDTQGLTPLHLAAAIGHLEIVEVLLKAGADVNAKDAYGETPLHLVASRGHLEIVEVLLKAGADVNAQDYSGRTPADLAAEYGHEDIAEVLQKLNGSPTPTPTTPTPTPTTPTPTPTGSDLGDKLLHAAYYGQDDEVRILLAAGADVNAKDTQGLTPLHLAAAIGHLEIVEVLLKAGADVNAKDAYGETPLHLVASRGHLEIVEVLLKAGADVNAQDYSGRTPADLAAEYGHEDIAEVLQKLNGSPTPTPTTPTPTPTTPTPTPTGSDLGDKLLHAAYYGQDDEVRILLAAGADVNAKDTQGLTPLHLAAAIGHLEIVEVLLKAGADVNAKDAYGETPLHLVASRGHLEIVEVLLKAGADVNAQDYSGRTPADLAAEYGHEDIAEVLQKLN

SEQ ID NO: 56 - protein, 422 aa, Artificial Sequence
  Protein comprising three ankyrin repeat domains
GSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKLNGSPTPTPTTPTPTPTTPTPTPTGSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKLNGSPTPTPTTPTPTPTTPTPTPTGSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKLN

SEQ ID NO: 57 - protein, 22 aa, Artificial Sequence
  PT linker
GSPTPTPTTPTPTPTTPTPTPT

SEQ ID NO: 58 - protein, 33 aa, Artificial Sequence
  Ankyrin repeat module
KDLRGITPLHVAAWQGHLEIVEVLLKAGADVNA

SEQ ID NO: 59 - protein, 33 aa, Artificial Sequence
  Ankyrin repeat module
KDSKGETPLHLAAFRGHLEIVEVLLKAGADVNA

SEQ ID NO: 60 - protein, 33 aa, Artificial Sequence
  Ankyrin repeat module
KDYHGYTPLHHAATYGHLEIVEVLLKAGADVNA

SEQ ID NO: 61 - protein, 33 aa, Artificial Sequence
  Ankyrin repeat module
KDQTGLTPLHLAAAKGHLEIVEVLLKAGADVNA

SEQ ID NO: 62 - protein, 33 aa, Artificial Sequence
  Ankyrin repeat module
KDSRGITPLHVAAWQGHLEIVEVLLKASADVNA

SEQ ID NO: 63 - protein, 33 aa, Artificial Sequence
  Ankyrin repeat module
KDHAGITPLHLAAFLGHLEIVEVLLKAGADVNA

SEQ ID NO: 64 - protein, 33 aa, Artificial Sequence
  Ankyrin repeat module
KDTYGETPLHLAAWKGHLEIVEVLLKAGADVNA

SEQ ID NO: 65 - protein, 33 aa, Artificial Sequence
  Ankyrin repeat module
KDSIGITPLHVAAHHGHLEIVEVLLKAGADVNA

SEQ ID NO: 66 - protein, 33 aa, Artificial Sequence
  Ankyrin repeat module
KDKWGRTPLHLAALLGHLEIAEVLLKAGADVNA

SEQ ID NO: 67 - protein, 34 aa, Artificial Sequence
  Ankyrin repeat module
KDQEHGSTPLHLAANYGHLEIVEVLLKAGADVNA

SEQ ID NO: 68 - protein, 33 aa, Artificial Sequence
  Ankyrin repeat module
KNWWGNTPLHLAAVYGHLEIVEVLLKAGADVNA

SEQ ID NO: 69 - protein, 33 aa, Artificial Sequence
  Ankyrin repeat module
KDNHGHTPLHLAAWYGHLEIVEVLLKAGADVNA

SEQ ID NO: 70 - protein, 33 aa, Artificial Sequence; identical to SEQ ID NO: 60
  Ankyrin repeat module

SEQ ID NO: 71 - protein, 33 aa, Artificial Sequence; identical to SEQ ID NO: 61
  Ankyrin repeat module

SEQ ID NO: 72 - protein, 28 aa, artificial sequence
  C-cap
QDKFGKTPADIAADNGHEDIAEVLQKLN

SEQ ID NO: 73 - protein, 28 aa, artificial sequence
  C-cap
QDQQGETPADLAALAGHVDIAEVLQKAA

SEQ ID NO: 74 - protein, 28 aa, Artificial Sequence
  C-cap
QDQQGETPADLAALAGHVDIAEVLQKLN

SEQ ID NO: 75 - protein, 28 aa, Artificial Sequence
  C-cap
  misc_feature (3)..(4): Xaa can be any naturally occurring amino acid
  misc_feature (6)..(6): Xaa can be any naturally occurring amino acid
  misc_feature (14)..(15): Xaa can be any naturally occurring amino acid
QDXXGXTPADLAAXXGHEDIAEVLQKLN

SEQ ID NO: 76 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 77 - protein, 254 aa, Macaca fascicularis
MGNSCYNIVATLLLVLNFERTRSLQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQIIFFLALTSTVVLFLLFFLVLRFSVVKRSRKKLLYIFKQPFMRPVQTTQEEDGCSCRFPEEEEGGCEL

SEQ ID NO: 78 - protein, 256 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 79 - protein, 163 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 80 - protein, 163 aa, Macaca fascicularis
LQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQ

SEQ ID NO: 81 - protein, 164 aa, Mus musculus
VQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVL